SEQUENCE LISTING (109 sequences)

SEQ ID NO: 1 - DNA, 702 bp, Simmondsia chinensis
  source 1..702: mol_type = genomic DNA
atggagactactactagtacggagatgaagaagagagaattaaagcatctagggtttgtggaggtggctgcggtgaacacgatcatgtgtctttcgagtgtttatgagtatgcaaaacagaattctgggccgttgaaatcaacagttggtgcagttgagggtgctgttaccactgtggtttcccctgtctatgagaaattcaagggcgttcctgatgatctccttggtttccttgacgaaaaggtaggtgtggcaagtgaaaaatttgacgagcacgctcctccattggccaagcatttggtatcaaaaactcatgatatcatcgtaaaggcatcccaagtgacccaagaccttatcaaccacgccaagactgatggccctcgcgctgctgcacattacgctgcctctgagtcgaagcagcttgtgtttattcaatccgcgaaaatctggaccaggctcgaccaaatcccgcccttccacatgctagcagacattgctgtccctacagccgctcactggtcagaaaagtacaataaagtcgtggttgacatgagcagtaagggccacgttatcttcagttaccttcccttggttcctatcgatgacattgccaaggcttttaagccggcgggctcagccaaagaggagggtggtgaagatggtgctgcaccggctcatgaagcgcagtcgcagtcggattga

SEQ ID NO: 2 - DNA, 723 bp, Arabidopsis thaliana
  source 1..723: mol_type = genomic DNA
atggagacagagaagaaaaatagcaaggaggtagcgctaaaacacctagcgttcgtgagaattgcaacgatccatattctggcttccgtctcaaatctctacgaatacgctaaacaaaactccggtcctctcaaatccgccgtggaaaaggtcgaaggagccgtcaccaccgtcgtcacccctgtctatcagaaattcaaagatgttcctgattctctcctggtctttctagatcacaaggtgggtgaagtttcgtacaagtttgatgagcatgctcctccaatggctaagaaagtggtgaatcaagcacatgtattgatctacaaggcgacagagaaagctcaaagctttgtgaaagaggctcgtaccggtggtcccaaagctgcgtttaactatgctgcaactgagtacaagtttttcgttgtgaccaactcggttaaagtctgggctaaacttaaccagtataaaccaatccatgcaatgggtgacaaagctttgcctgtggctgcacacttttccagtcggtacaacgatttggtgactgatatgactaatatgggttactctttggttggttatcttccgttggttcctgttgatgacattgttaaggcttatgaaaaggaagatgcaaggagaaagaaaggaggggatacggctggaaagaaaggagagactactgatgcggctgatggtgataaatcatcgtctgattcggagtag

SEQ ID NO: 3 - DNA, 693 bp, unidentified
  source 1..693: mol_type = genomic DNA
atggcgaccaacaaggttgagatggagaaaagtgagggaaatttgaaacatctagggtttgtaagggtgttggctataaacactgcggttttggtgtcaaatctgtatgaatacacgaagcagaactcgggtcctctgagatcgactgtgggtactgtagagaatgcggtaactaccgtggtaaggcctgtttacgagaggcttaaaggtgttcctgatgaagtccttgttttcctagaccagaaggtggacgatggagcagcaaaatttgatgagcatgctcctcccttggccaagaaggttgtcagcaaagcccagtctgtatttcagaaggcatcagaagtagcacaagacttgttcaaggaagtacaagttgctggtcctcgtgcagctatcaatcatgctggtgcattgtccaagcaattggctgctagtcaagtggcagttctctggtatcatataaatcattgtgcgccgttgcatggaatcgcacagatggctgctcctactgctgctcactggtcagagaagtataatcaactagttgctgacttgaagaagaaaggttatattgttgtcaactacattcctttgataccagttgaagaaatctcgaaggcatataaacaggttgagtctgctgcggatgagaaagacgatgctcctaattccagttcaagtaaatccgaatga

SEQ ID NO: 4 - DNA, 699 bp, synthetic construct
  source 1..699: mol_type = other DNA
atggagacagagaagaaaaatagcaaggaggtagcgctaaaacacctagcgttcgtgagaattgcaacgatccatattctggcttccgtctcaaatctctacgaatacgctaaacaaaactccggtcctctcaaatccgccgtggaaaaggtcgaaggagccgtcaccaccgtcgtcacccctgtctatcagaaattcaaagatgttcctgattctctcctggtctttctagatcacaaggtgggtgaagtttcgtacaagtttgatgagcatgctcctccaatggctaagaaagtggtgaatcaagcacatgtattgatctacaaggcgacagagaaagctcaaagctttgtgaaagaggctcgtaccgatggccctcgcgctgctgcacattacgctgcctctgagtcgaagcagcttgtgtttattcaatccgcgaaaatctggaccaggctcgaccaaatcccgcccttccacatgctagcagacattgctgtccctacagccgctcactggtcagaaaagtacaataaagtcgtggttgacatgagcagtaagggccacgttatcttcagttaccttcccttggttcctatcgatgacattgccaaggcttttaagccggcgggctcagccaaagaggagggtggtgaagatggtgctgcaccggctcatgaagcgcagtcgcagtcggattga

SEQ ID NO: 5 - DNA, 726 bp, synthetic construct
  source 1..726: mol_type = other DNA
atggagactactactagtacggagatgaagaagagagaattaaagcatctagggtttgtggaggtggctgcggtgaacacgatcatgtgtctttcgagtgtttatgagtatgcaaaacagaattctgggccgttgaaatcaacagttggtgcagttgagggtgctgttaccactgtggtttcccctgtctatgagaaattcaagggcgttcctgatgatctccttggtttccttgacgaaaaggtaggtgtggcaagtgaaaaatttgacgagcacgctcctccattggccaagcatttggtatcaaaaactcatgatatcatcgtaaaggcatcccaagtgacccaagaccttatcaaccacgccaagactggtggtcccaaagctgcgtttaactatgctgcaactgagtacaagtttttcgttgtgaccaactcggttaaagtctgggctaaacttaaccagtataaaccaatccatgcaatgggtgacaaagctttgcctgtggctgcacacttttccagtcggtacaacgatttggtgactgatatgactaatatgggttactctttggttggttatcttccgttggttcctgttgatgacattgttaaggcttatgaaaaggaagatgcaaggagaaagaaaggaggggatacggctggaaagaaaggagagactactgatgcggctgatggtgataaatcatcgtctgattcggagtag

SEQ ID NO: 6 - DNA, 702 bp, synthetic construct
  source 1..702: mol_type = other DNA
atggagacagagaagaaaaatagcaaggaggtagcgctaaaacacctagcgttcgtgagaattgcaacgatccatattctggcttccgtctcaaatctctacgaatacgctaaacaaaactccggtcctctcaaatccgccgtggaaaaggtcgaaggagccgtcaccaccgtcgtcacccctgtctatcagaaattcaaagatgttcctgattctctcctggtctttctagatcacaaggtgggtgaagtttcgtacaagtttgatgagcatgctcctccaatggctaagaaagtggtgaatcaagcacatgtattgatctacaaggcgacagagaaagctcaaagctttgtgaaagaggctcgtaccggtggtcccaaagctgcgtttaactatgctgcaactgagtacaagtttttcgttgtgaccaactcggttaaagtctgggctaaacttaaccagtatatcccgcccttccacatgctagcagacattgctgtccctacagccgctcactggtcagaaaagtacaataaagtcgtggttgacatgagcagtaagggccacgttatcttcagttaccttcccttggttcctatcgatgacattgccaaggcttttaagccggcgggctcagccaaagaggagggtggtgaagatggtgctgcaccggctcatgaagcgcagtcgcagtcggattga

SEQ ID NO: 7 - DNA, 723 bp, synthetic construct
  source 1..723: mol_type = other DNA
atggagactactactagtacggagatgaagaagagagaattaaagcatctagggtttgtggaggtggctgcggtgaacacgatcatgtgtctttcgagtgtttatgagtatgcaaaacagaattctgggccgttgaaatcaacagttggtgcagttgagggtgctgttaccactgtggtttcccctgtctatgagaaattcaagggcgttcctgatgatctccttggtttccttgacgaaaaggtaggtgtggcaagtgaaaaatttgacgagcacgctcctccattggccaagcatttggtatcaaaaactcatgatatcatcgtaaaggcatcccaagtgacccaagaccttatcaaccacgccaagactgatggccctcgcgctgctgcacattacgctgcctctgagtcgaagcagcttgtgtttattcaatccgcgaaaatctggaccaggctcgaccaaaaaccaatccatgcaatgggtgacaaagctttgcctgtggctgcacacttttccagtcggtacaacgatttggtgactgatatgactaatatgggttactctttggttggttatcttccgttggttcctgttgatgacattgttaaggcttatgaaaaggaagatgcaaggagaaagaaaggaggggatacggctggaaagaaaggagagactactgatgcggctgatggtgataaatcatcgtctgattcggagtag

SEQ ID NO: 8 - DNA, 699 bp, synthetic construct
  source 1..699: mol_type = other DNA
atggagacagagaagaaaaatagcaaggaggtagcgctaaaacacctagcgttcgtgagaattgcaacgatccatattctggcttccgtctcaaatctctacgaatacgctaaacaaaactccggtcctctcaaatccgccgtggaaaaggtcgaaggagccgtcaccaccgtcgtcacccctgtctatcagaaattcaaagatgttcctgattctctcctggtctttctagatcacaaggtgggtgaagtttcgtacaagtttgatgagcatgctcctccaatggctaagaaagtggtgaatcaagcacatgtattgatctacaaggcgacagagaaagctcaaagctttgtgaaagaggctcgtaccggtggtcccaaagctgcgtttaactatgctgcaactgagtacaagtttttcgttgtgaccaactcggttaaagtctgggctaaacttaaccagtataaaccaatccatgcaatgggtgacaaagctttgcctgtggctgcacacttttccagtcggtacaacgatttggtgactgatatgactaatatgggttactctttggttggttatcttcccttggttcctatcgatgacattgccaaggcttttaagccggcgggctcagccaaagaggagggtggtgaagatggtgctgcaccggctcatgaagcgcagtcgcagtcggattga

SEQ ID NO: 9 - DNA, 726 bp, synthetic construct
  source 1..726: mol_type = other DNA
atggagactactactagtacggagatgaagaagagagaattaaagcatctagggtttgtggaggtggctgcggtgaacacgatcatgtgtctttcgagtgtttatgagtatgcaaaacagaattctgggccgttgaaatcaacagttggtgcagttgagggtgctgttaccactgtggtttcccctgtctatgagaaattcaagggcgttcctgatgatctccttggtttccttgacgaaaaggtaggtgtggcaagtgaaaaatttgacgagcacgctcctccattggccaagcatttggtatcaaaaactcatgatatcatcgtaaaggcatcccaagtgacccaagaccttatcaaccacgccaagactgatggccctcgcgctgctgcacattacgctgcctctgagtcgaagcagcttgtgtttattcaatccgcgaaaatctggaccaggctcgaccaaatcccgcccttccacatgctagcagacattgctgtccctacagccgctcactggtcagaaaagtacaataaagtcgtggttgacatgagcagtaagggccacgttatcttcagttaccttccgttggttcctgttgatgacattgttaaggcttatgaaaaggaagatgcaaggagaaagaaaggaggggatacggctggaaagaaaggagagactactgatgcggctgatggtgataaatcatcgtctgattcggagtag

SEQ ID NO: 10 - DNA, 702 bp, synthetic construct
  source 1..702: mol_type = other DNA
atggagactactactagtacggagatgaagaagagagaattaaagcatctagggtttgtggaggtggctgcggtgaacacgatcatgtgtctttcgagtgtttatgagtatgcaaaacagaattctgggccgttgaaatcaacagttggtgcagttgagggtgctgttaccactgtggtttcccctgtctatgagaaattcaagggcgttcctgatgatctccttggtttccttgacgaaaaggtaggtgtggcaagtgaaaaatttgacgagcacgctcctccattggccaagcatttggtatcaaaaactcatgatatcatcgtaaaggcatcccaagtgacccaagaccttatcaaccacgccaagactgatggccctcgcgctgctgcacattacgctgcctctgagtcgaagcagcttgtgtttattcaatccgcgaaaatctggaccaggctcgaccaaatcccgcccattcacatgctagcagacattgctgtccctacagccgctcactggtcagaaaagtacaataaagtcgtggttgacatgagcagtaagggccacgttatcttcagttaccttcccttggttcctatcgatgacattgccaaggcttttaagccggcgggctcagccaaagaggagggtggtgaagatggtgctgcaccggctcatgaagcgcagtcgcagtcggattga

SEQ ID NO: 11 - DNA, 702 bp, synthetic construct
  source 1..702: mol_type = other DNA
atggagactactactagtacggagatgaagaagagagaattaaagcatctagggtttgtggaggtggctgcggtgaacacgatcatgtgtctttcgagtgtttatgagtatgcaaaacagaattctgggccgttgaaatcaacagttggtgcagttgagggtgctgttaccactgtggtttcccctgtctatgagaaattcaagggcgttcctgatgatctccttggtttccttgacgaaaaggtaggtgtggcaagtgaaaaatttgacgagcacgctcctccattggccaagcatttggtatcaaaaactcatgatatcatcgtaaaggcatcccaagtgacccaagaccttatcaaccacgccaagactgatggccctcgcgctgctgcacattacgctgcctctgagtcgaagcagcttgtgtttattcaatccgcgaaaatctggaccaggctcgaccaaatcccgcccttccacatgatggcagacattgctgtccctacagccgctcactggtcagaaaagtacaataaagtcgtggttgacatgagcagtaagggccacgttatcttcagttaccttcccttggttcctatcgatgacattgccaaggcttttaagccggcgggctcagccaaagaggagggtggtgaagatggtgctgcaccggctcatgaagcgcagtcgcagtcggattga

SEQ ID NO: 12 - DNA, 702 bp, synthetic construct
  source 1..702: mol_type = other DNA
atggagactactactagtacggagatgaagaagagagaattaaagcatctagggtttgtggaggtggctgcggtgaacacgatcatgtgtctttcgagtgtttatgagtatgcaaaacagaattctgggccgttgaaatcaacagttggtgcagttgagggtgctgttaccactgtggtttcccctgtctatgagaaattcaagggcgttcctgatgatctccttggtttccttgacgaaaaggtaggtgtggcaagtgaaaaatttgacgagcacgctcctccattggccaagcatttggtatcaaaaactcatgatatcatcgtaaaggcatcccaagtgacccaagaccttatcaaccacgccaagactgatggccctcgcgctgctgcacattacgctgcctctgagtcgaagcagcttgtgtttattcaatccgcgaaaatctggaccaggctcgaccaaatcccgcccattcacatgatggcagacattgctgtccctacagccgctcactggtcagaaaagtacaataaagtcgtggttgacatgagcagtaagggccacgttatcttcagttaccttcccttggttcctatcgatgacattgccaaggcttttaagccggcgggctcagccaaagaggagggtggtgaagatggtgctgcaccggctcatgaagcgcagtcgcagtcggattga

SEQ ID NO: 13 - DNA, 723 bp, synthetic construct
  source 1..723: mol_type = other DNA
atggagacagagaagaaaaatagcaaggaggtagcgctaaaacacctagcgttcgtgagaattgcaacgatccatattctggcttccgtctcaaatctctacgaatacgctaaacaaaactccggtcctctcaaatccgccgtggaaaaggtcgaaggagccgtcaccaccgtcgtcacccctgtctatcagaaattcaaagatgttcctgattctctcctggtctttctagatcacaaggtgggtgaagtttcgtacaagtttgatgagcatgctcctccaatggctaagaaagtggtgaatcaagcacatgtattgatctacaaggcgacagagaaagctcaaagctttgtgaaagaggctcgtaccggtggtcccaaagctgcgtttaactatgctgcaactgagtacaagtttttcgttgtgaccaactcggttaaagtctgggctaaacttaaccagtataaaccattccatgcaatgggtgacaaagctttgcctgtggctgcacacttttccagtcggtacaacgatttggtgactgatatgactaatatgggttactctttggttggttatcttccgttggttcctgttgatgacattgttaaggcttatgaaaaggaagatgcaaggagaaagaaaggaggggatacggctggaaagaaaggagagactactgatgcggctgatggtgataaatcatcgtctgattcggagtag

SEQ ID NO: 14 - DNA, 723 bp, synthetic construct
  source 1..723: mol_type = other DNA
atggagacagagaagaaaaatagcaaggaggtagcgctaaaacacctagcgttcgtgagaattgcaacgatccatattctggcttccgtctcaaatctctacgaatacgctaaacaaaactccggtcctctcaaatccgccgtggaaaaggtcgaaggagccgtcaccaccgtcgtcacccctgtctatcagaaattcaaagatgttcctgattctctcctggtctttctagatcacaaggtgggtgaagtttcgtacaagtttgatgagcatgctcctccaatggctaagaaagtggtgaatcaagcacatgtattgatctacaaggcgacagagaaagctcaaagctttgtgaaagaggctcgtaccggtggtcccaaagctgcgtttaactatgctgcaactgagtacaagtttttcgttgtgaccaactcggttaaagtctgggctaaacttaaccagtataaaccaatccatgcacttggtgacaaagctttgcctgtggctgcacacttttccagtcggtacaacgatttggtgactgatatgactaatatgggttactctttggttggttatcttccgttggttcctgttgatgacattgttaaggcttatgaaaaggaagatgcaaggagaaagaaaggaggggatacggctggaaagaaaggagagactactgatgcggctgatggtgataaatcatcgtctgattcggagtag

SEQ ID NO: 15 - DNA, 723 bp, synthetic construct
  source 1..723: mol_type = other DNA
atggagacagagaagaaaaatagcaaggaggtagcgctaaaacacctagcgttcgtgagaattgcaacgatccatattctggcttccgtctcaaatctctacgaatacgctaaacaaaactccggtcctctcaaatccgccgtggaaaaggtcgaaggagccgtcaccaccgtcgtcacccctgtctatcagaaattcaaagatgttcctgattctctcctggtctttctagatcacaaggtgggtgaagtttcgtacaagtttgatgagcatgctcctccaatggctaagaaagtggtgaatcaagcacatgtattgatctacaaggcgacagagaaagctcaaagctttgtgaaagaggctcgtaccggtggtcccaaagctgcgtttaactatgctgcaactgagtacaagtttttcgttgtgaccaactcggttaaagtctgggctaaacttaaccagtataaaccattccatgcacttggtgacaaagctttgcctgtggctgcacacttttccagtcggtacaacgatttggtgactgatatgactaatatgggttactctttggttggttatcttccgttggttcctgttgatgacattgttaaggcttatgaaaaggaagatgcaaggagaaagaaaggaggggatacggctggaaagaaaggagagactactgatgcggctgatggtgataaatcatcgtctgattcggagtag

SEQ ID NO: 16 - DNA, 1059 bp, Simmondsia chinensis
  source 1..1059: mol_type = genomic DNA
atggaggtggagaaggagctagagaccttcgcagaggtatggatctccgccatagccgccgcctgctactgccgcttcgtccccgccgttgcccctcacggcggcgctctccgcctcctcctcctcctccccgtcgtcctcctcttcattttcctccccctccgcctctcctccttccacctcggcggccccaccgccttgtatctcgtctggcttgccaacttcaagctccttctcttcgcctttcatcttggccctttatctaacccctctctctctctccttcacttcatctccaccaccctcctccccatcaagttcagagatgacccatctaatgatcatgagaaaaacaagagaactctgagttttgagtggcgtaaagttgttctttttgttgctaagttggtgttttttgcgggtattttaaagatttatgagtttagaaaagatttgcctcattttgtgatctcggtgctttactgttttcacttctatctcgggacggagatcaccttagcagcaagcgcagtcatagctcgagccacgctagggttagacctatacccccagttcaacgagccatacttagccacctcgctgcaagacttctgggggcgcaggtggaacctcatggtgtcagacatcttggggttgacaacataccagcctgtccggcgtgtcctctcgaggtgggtcaggctgcggtgggaggtcgccggcgcaatgttggtggcgttcacggtgtcggggctaatgcatgaagtgtttttcttctacttaactcgcgcgaggccctcgtgggaggtgacggggttctttgtgttgcatggggtttgcacagccgtggagatggtggtgaagaaggcggtttcaggcaaggtgcggctgcgccgggaggtgtcaggggcgctgacggtggggttcgtgatggtgactggagggtggttgtttttgccgcagctggtgaggcatggggtagatttgaagaccattgatgagtatcctgtcatgtttaattatactcagaagaaattgatgggtttgttggggtggtga

SEQ ID NO: 17 - DNA, 1482 bp, Simmondsia chinensis
  source 1..1482: mol_type = genomic DNA
atggaggaaatgggaagcattttagagtttcttgataacaaagccattttggtcactggtgctactggctccttagcaaaaatttttgtggagaaggtactgaggagtcaaccgaatgtgaagaaactctatcttcttttgagagcaaccgatgacgagacagctgctctacgcttgcaaaatgaggtttttggaaaagagttgttcaaagttctaaaacaaaatttaggtgcaaatttctattcctttgtatcagaaaaagtgactgtagtacccggtgatattactggtgaagacttgtgtctcaaagacgtcaatttgaaggaagaaatgtggagggaaatcgatgttgttgtcaatctagctgctacaatcaacttcattgaaaggtacgacgtgtctctgcttatcaacacatatggagccaagtatgttttggacttcgcgaagaagtgcaacaaattaaagatatttgttcatgtatctactgcttatgtatctggagagaaaaatgggttaatactggagaagccttattatatgggcgagtcacttaatggaagattaggtctggacattaatgtagagaagaaacttgtggaggcaaaaatcaatgaacttcaagcagcgggggcaacggaaaagtccattaaatcgacaatgaaggacatgggcatcgagagggcaagacactggggatggccaaatgtgtatgtattcaccaaggcattaggggagatgcttttgatgcaatacaaaggggacattccgcttactattattcgtcccaccatcatcaccagcacttttaaagagccctttcctggttgggttgaaggtgtcaggaccatcgataatgtacctgtatattatggtaaagggagattgaggtgtatgctttgcggacccagcacaacaattgacctgataccggcagatatggtcgtgaatgcaacgatagtagccatggtggcgcacgcaaaccaaagatacgtagagccggtgacataccatgtgggatcttcagcggcgaatccaatgaaactgagtgcattaccagagatggcacaccgttacttcaccaagaatccatggatcaacccggatcgcaacccagtacatgtgggtcgggctatggtcttctcctccttctccaccttccacctttatctcacccttaatttcctccttcctttgaaggtactggagatagcaaatacaatattctgccaatggttcaagggtaagtacatggatcttaaaaggaagacgaggttgttgttgcgtttagtagacatttataaaccctacctcttcttccaaggcatctttgatgacatgaacactgagaagttgcggattgctgcaaaagaaagcatagttgaagctgatatgttttactttgatccgaaggcaattaactgggaagattacttcttgaaaactcatttcccaggtgtcgtagagcacgttcttaactaa

SEQ ID NO: 18 - DNA, 1566 bp, Simmondsia chinensis
  source 1..1566: mol_type = genomic DNA
atgaaggccaaaacaatcacaaacccggagatccaagtctccacgaccatgaccaccacgaccacgaccgccactctccccaacttcaagtcctccatcaacttacaccacgtcaagctcggctaccactacttaatctccaatgccctcttcctcgtattcatcccccttttgggcctcgcttcggcccatctctcctccttctcggcccatgacttgtccctgctcttcgacctccttcgccgcaacctcctccctgttgtcgtttgttctttcctcttcgttttattagcaaccctacatttcttgacccggcccaggaatgtctacttggtggactttgcatgctataagcctcacccgaacctgatgacatcccacgagatgttcatggaccggacctcccgggccgggtcgttttctaaggagaatattgagtttcagaggaagatcttggagagggccggtatgggccgggaaacctacgtccccgaatccgtcactaaggtgccgcccgagccgagcatagcagcagccagggccgaggcggaggaggtgatgtacggggcgatcgacgaggtgttggagaagacgggggtgaagccgaagcagataggaatactggtggtgaactgcagcttgtttaacccaacgccgtcgctgtcatccatgatagttaaccattacaagcttaggggtaatatacttagctataatcttggtggcatgggttgcagtgctgggctcatttccattgatcttgccaaggacctcctacaggtttaccgtaacacatatgtgttagtagtgagcacagaaaacatgacccttaattggtactggggcaatgaccgctccatgcttatcaccaactgcctatttcgcatgggtggcgctgccatcatcctctcaaaccgctggcgtgatcgtcgccgatccaagtaccaactccttcacacagtacgcacccacaagggcgctgacgacaagtcctatagatgcgtcttacaacaagaagatgaaaataacaaggtaggtgttgccttatccaaggatctgatggcagttgccggtgaagccctaaaggccaacatcacgacccttggtcccctcgtgctccccatgtcagaacaactcctcttctttgccaccttagtggcacgtaaggtcttcaagatgacgaacgtgaagccatacatcccagatttcaagttggcagcgaagcacttctgcatccatgcaggaggcaaagcagtgttggatgagctcgagacgaacttggagttgacgccatggcaccttgaaccctcgaggatgacactgtataggtttgggaacacatcgagtagctcattatggtacgagttggcatacgctgaagcaaaagggaggatccgtaagggtgatcgaacttggatgattggatttggttcaggtttcaagtgtaacagtgttgtgtggagggctttgaggagtgtcaatccggctagagagaagaatccttggatggatgaaattgagaatttccctgtccatgtgcctaaaatcgcacctatcgcttcgtag

SEQ ID NO: 19 - DNA, 405 bp, Simmondsia chinensis
  source 1..405: mol_type = genomic DNA
atggcggagtacggcggcgggcagcaaccagggcagcagcctaaggcccaacaggtggctaaggccgtaaccgcggtcacggccggaggttcgctcttggtcctcgccggactaatcctcgctgcgactgtgatcggcctcaccattgccacccctttactggtaatattcagcccagtcctcgtcccagccgtgatcacagctgtgttgatcgtagggggatttctaacgtccggtgggtttgggttggcagctatttcggtgttatcgtggatttacaggtacgtgacgggaaaacacccgcctggggctgaccaactcgagcaagcacggatgaagctggcgggtaagacgagggaggctaccgaatacgttggtcaacagattcagcaggagagtacttaa

SEQ ID NO: 20 - DNA, 747 bp, Simmondsia chinensis
  source 1..747: mol_type = genomic DNA
atgaaacttgttatgcatgcacacctcacgttcaacacctgtccctccacgtttctctctctcttctctatatattcccaccgccaccacctctaccacctaccgccgccgtcaaccaccacaacatcccactttctctcccttacttccaccaccaactctatctccgccatggccgaaacccgagaccgcagtgaccgcgaccgtggagaccgtgaccgtgacattgctgtccggccccaccaccagctccaagtccactctcaaaagggtggggggactctcagctatggccgaggagccgccaccggcagtggtaggggccccactgcatctcaagtcatagcggtggtgaccctggttccgattggtggaatactgttgtgtctggctgggttcacactgttgggatccctaatagggttagcagtggcaaccccgctgtttatattattcagtccagttcttgttcctgcaatcatactgatagccttggcggtcactgggttcctatcgtcaggggcgtttggactgacagggctatcatcgttgtcgtgggtggcggggtatttaaagcaagcgagccagagggtgccagaggacctggactacgcaaagaagagaatggcggatgtggcagcgtacgcagggcagaagacaaaggaggcggggcacactgtagagagtaaggccaaggagactgggacgggcacaactacaagcacagggactacaggccggacctga

SEQ ID NO: 21 - DNA, 519 bp, Simmondsia chinensis
  source 1..519: mol_type = genomic DNA
atggctgactaccgtgaccgcgaccgtgacaccactggccgccactacccctaccagcaggctggctacaagggcagcacccttggctaccgtggcggtgctggcggtggcagtggcggtggcccctcggcctcccaagtcatagcagtcatcaccctcctccccgtcagcgggaccctcctcgtcctggccgggcttgctcttgcagggtcgataatagggttgatagtcaccaccccgttgttcatcctcttcagcccggtcctggtgcctgctgccctgctaattgggcttgctgtgacgggattcttgtcatcaggtgcgtttgggctcacggggctgtcagcgctgtcgagggtggtggcctaccttaggcaggcggcacagagtatgccggaggacctggattacgcgaagcggcgagctgcagacgccggggcatacgcggggcagaagacgaaggaggccggacagaccatagaggggaagccacgtgagactgggactaccaggacttga

SEQ ID NO: 22 - DNA, 705 bp, Simmondsia chinensis
  source 1..705: mol_type = genomic DNA
atggagaaaatggaggactcgtatgaaccagacaagcccaacggaggttctacggtgaccgaagacgacggttacactcccaccaccaagcacacccgcttcgatggcggaggagaaagtctttacgacgtcgttacggatcttgtctcttcgatcttgtcccctgatcctcacagcagtttagctgcctcgtcactgctccatcgcacgaaggtttccttgaaagagaaccttccgcgccttccagaagcttctagaagcaccgggttgaggatccttcggtgggcccgccgtggcagccctcttcggttgctccttgttatatctgttggaacaatcactctcctgaccttaacagggctgttagttttcatgctctttttggtggctgcaactgtcaatgcgattgtcctgtctcttatcatgtctttggtggctgctggaggcttcttggctattttctttgcatgtgtggcagctatttatattgggacattatcagttgccgttttcgtcatttcttctgcagcaatatcagcaattatagctgtcgctatagctgcaggttggattggtttcctctggactgtatggtttaccataaggaaaagtgcaggttttgccaagcactcaatacacgtgactggttcaactctttcagcttacactgctagttggcaggctcaccgccagcatcacgattga

SEQ ID NO: 23 - DNA, 750 bp, Simmondsia chinensis
  source 1..750: mol_type = genomic DNA
atggacacccaacttgaacaacaaatgaagggtgaaaatgcagagcaaaaactgaagtaccttcagttcgttcatgtcgctgcccttcatgctgttgtgtgcttctccaacctctatgtgtatgccaaggataaagctggtcctctgaaacctggggtcgtgactgttgagggaacggtgaagactgttgttggtcctgtgtatgataagttccatgatgttccctttgaactcctcaagtttgttgatcgtaagatggaggagtcgataaccaagttggagcaccatgtcccaccagtggttaaacaggtctccacccaagctgtctccgcagcccaaaaggctccagaggtggctcgcactgtcacctccgaagtccaacgcaccggcgttgtcgacactgcagcaggtatcgcaagaaacttctacaccaaatatgagcccacggccaaagagctctactccaaatacgagcccgtagctgagcagtacgtggtgctggcttggcagtcactgaaccggcttcccatattcccacaagtggctcaggtggtggttccgaaggcagcctactgttccgagaagtacaaccagacggtccggtccacggccgagaagggatactgggtggcgaactatttgcccttggtcccaactcagatgattgctgaagtgttcagagcccagaatgatgatgaatacaagcctctggattctaatggtgaagatgctgctgttgctgtgcaatga

SEQ ID NO: 24 - DNA, 1140 bp, Simmondsia chinensis
  source 1..1140: mol_type = genomic DNA
atggaagtcaacaatttctactccaaccgccagttttctcccaccaaaacttttcccttcaatttcatcaacccaatactccaactcctccaccattcccctcttcccggccctgccgtcatccgccgccacacacccaccgcgcggcagccgctgctgaggcgggtggcaaatggggcgatcggggcagcgtacacatgcatggcgatggcagcggtgatggggctggcggtgttggtgggggtggcggcagtgaacacgtgggtcgaggagccagtggcggtgagagagaaggtgcagtttgattacagggagatgaaccctcaggctctgtttgtgtttgaggggaggaggattccagttgagcatagagtttatgtttctttgatgcttttgttgcctgagtctgattacaacagagacactggcgtttttcagataaccacggaagtaatctcaacggaagggaaaacaatcacgcagtccagcaggccaacaatgctacacttccggagccacccgatcaggattgcacggactttctcaatgggcctgcccctcttattaggcctagcattggaggcccaaacaatcaaggtccaactcctaaagtacaaggaaagcaagctcttacgtacatccgccatcgaactcaccctctcacctcgagcaggaaccacctacctcccgcaactctatgaggccgagatcataattcactcccatcctccgtggagcaaagatttcgtgcgaaagtggaagttgactttctacgtgtggacgtctttatatgtcttcctgatcctcctcgttgttatcatttgcttctgcaggccattgattttaccagatggcaggtcgggattgaacggtcggggggtggggagagagtccagggcagtggtggaggtaaggaggaagcgtgtggtgggggaagggagagagttctcagagacactaaggacgtggcagcagtacaggcggagaaggagggcagaacttctaagtagtaggcaggagttggtggctgccaccactggatccgcctccagctacaccattacgaaggggttagagagttatgggggtggcaccaaggaggacgtaggggactcggagtcggtgtgtatgggaggttga

SEQ ID NO: 25 - DNA, 1533 bp, Simmondsia chinensis
  source 1..1533: mol_type = genomic DNA
atggagaattcacaatcatccgatgaagacgatcccttcttcgacgcacctgacgatttcctcttctttgattgcttcgaaagcttcgattcaactcgatattcgtctctttctctctcctcacgtgccgatcacacgtacagcctcaatccgccggaatcgtcctcttccggtggcctccgccgccgccgatccttctcccaccgccacggccgccatggctccggtgatgctttcgaggattcgagaatgcggaaattggaagatgaggagagagaaagtaagaaaccgaaagatgaggagagagaaagtagggaacttggggaagggagattggtagaaagtgtaagtgaagagaagggagagaattcaacggtgacaatagaggaaaaggagcagaatggtgaagttacaagtgcaaatgatgatgaaaggcaatttaattgtctgattttcatagcagaattgatatttaaagcaattgggtttcaatttaatctgtttatcagttttttcaccttacccatgaaaggaatttattattctgtgatgtttgtgatgaacccatttcaaagtttgagtcttggtagggattatgtgattagaaaattgatggggttatggaagtttgtatgtccatctctatctgactggttaaatgaacacaaatcatttttgaatgtcgtgttccggtgtggttgggggttgttttggtcagtttatgtgtgcattgttcttgtgattttgttgatttcagcatttatgataagtggggtcatgatgaggtgtgtggtggaggagccatttcaattgaagcaggcgttgaattttgattacactaaggacagtcctgttgtgtttgttccgatcacgtcttgttctagtaccccttgcttagattgtgtggaggataatgagtttggagatgtgagaggagtgacgattatacctcccaatcacaagttgcaggccacggtgacattgattttgccggagtctgattacaatcggaatctgggaatctttcaggttagggtagagttcctatccgcgaacggtaaacctcttgcaagtctaagccatccatgcatgttgaagttcaagagtgatcccatccgtttactactaacgctattcaaagtcatcccacttgttgctgggtacatatccgagtcccaaactctaaacttaaaatttaaaggattcactgaacgagatgtccatactgcctgcttgaaagtggcgatcgaacagagagcagagttccgccctgctggtggtattcctgaagtatacgatgcatttttactgcttgaatcgcaacttcccttcctcaagagaatcatttggtgctggaagaaaactatttttgtttggctcagtatgatgctgtttatgatgcagttgctgtttgtgttggtttgttgcagacccatcatcctcccgagagctagatcacggcgtggtacttctagaaccatctctgctgaaacgggtcagccagttcaaaggatgaactactggtacggctga

SEQ ID NO: 26 - protein, 233 aa, Simmondsia chinensis
METTTSTEMKKRELKHLGFVKVAAVNTIMCLSSVYEYAKQNSGPLKSTVGAVEGAVTTVVSPVYEKFKGVPDDLLGFLDEKVGVASEKFDEHAPPLAKHLVSKTHDIIVKASQVTQDLINHVKTDGPRAAAHYAASESKQLVFIQSAKIWTRLDQIPPFHMLADIAVPTAAHWSEKYNKVVVDMSCKGHVIFSYLPLVPIDDIAQAFKPAGSAKEEEGEDGAAPAHEAQSQSD

SEQ ID NO: 27 - protein, 240 aa, Arabidopsis thaliana
METEKKNSKEVALKHLAFVRIATIHILASVSNLYEYAKQNSGPLKSAVEKVEGAVTTVVTPVYQKFKDVPDSLLVFLDHKVGEVSYKFDEHAPPMAKKVVNQAHVLIYKATEKAQSFVKEARTGGPKAAFNYAATEYKFFVVTNSVKVWAKLNQYKPIHAMGDKALPVAAHFSSRYNDLVTDMTNMGYSLVGYLPLVPVDDIVKAYEKEDARRKKGGDTAGKKGETTDAADGDKSSSDSE

SEQ ID NO: 28 - DNA, 8193 bp, synthetic construct
  source 1..8193: mol_type = other DNA
gaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcctgtacccccgcgacggcgtgctgaagggcgagacacacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatgaaggccaaaacaatcacaaacccggagatccaagtctccacgaccatgaccaccacgaccacgaccgccactctccccaacttcaagtcctccatcaacttacaccacgtcaagctcggctaccactacttaatctccaatgccctcttcctcgtattcatcccccttttgggcctcgcttcggcccatctctcctccttctcggcccatgacttgtccctgctcttcgacctccttcgccgcaacctcctccctgttgtcgtttgttctttcctcttcgttttattagcaaccctacatttcttgacccggcccaggaatgtctacttggtggactttgcatgctataagcctcacccgaacctgatgacatcccacgagatgttcatggaccggacctcccgggccgggtcgttttctaaggagaatattgagtttcagaggaagatcttggagagggccggtatgggccgggaaacctacgtccccgaatccgtcactaaggtgccgcccgagccgagcatagcagcagccagggccgaggcggaggaggtgatgtacggggcgatcgacgaggtgttggagaagacgggggtgaagccgaagcagataggaatactggtggtgaactgcagcttgtttaacccaacgccgtcgctgtcatccatgatagttaaccattacaagcttaggggtaatatacttagctataatcttggtggcatgggttgcagtgctgggctcatttccattgatcttgccaaggacctcctacaggtttaccgtaacacatatgtgttagtagtgagcacagaaaacatgacccttaattggtactggggcaatgaccgctccatgcttatcaccaactgcctatttcgcatgggtggcgctgccatcatcctctcaaaccgctggcgtgatcgtcgccgatccaagtaccaactccttcacacagtacgcacccacaagggcgctgacgacaagtcctatagatgcgtcttacaacaagaagatgaaaataacaaggtaggtgttgccttatccaaggatctgatggcagttgccggtgaagccctaaaggccaacatcacgacccttggtcccctcgtgctccccatgtcagaacaactcctcttctttgccaccttagtggcacgtaaggtcttcaagatgacgaacgtgaagccatacatcccagatttcaagttggcagcgaagcacttctgcatccatgcaggaggcaaagcagtgttggatgagctcgagacgaacttggagttgacgccatggcaccttgaaccctcgaggatgacactgtataggtttgggaacacatcgagtagctcattatggtacgagttggcatacgctgaagcaaaagggaggatccgtaagggtgatcgaacttggatgattggatttggttcaggtttcaagtgtaacagtgttgtgtggagggctttgaggagtgtcaatccggctagagagaagaatccttggatggatgaaattgagaatttccctgtccatgtgcctaaaatcgcacctatcgcttcgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggtggagaaggagctagagactttcgcagaggtatggatctccgccatagccgccgcctgctactgccgcttcgtccccgccgttgcccctcacggcggcgctctacgcctcctcctcctcctccccgtcgtcctcctcttcattttcctccccctccgcctctcctccttccacctcggcggccccaccgccttgtatctcgtctggcttgccaacttcaagctccttctcttcgcctttcatcttggccctttatctaacccctctctctctctccttcacttcatctccaccaccctcctccccatcaagttcagagatgacccatctaatgatcatgagaaaaacaagagaactctgagttttgagtggcgtaaagttgttctttttgttgctaagttggtgttttttgcgggtattttaaagatttatgagtttagaaaagatttgcctcattttgtgatctcggtgctttactgttttcacttctatctcgggacggagatcaccttagcagcaagcgcagtcatagctcgagccacgctagggttagacctatacccccagttcaacgagccatacttagccacctcgctgcaagacttctgggggcgcaggtggaacctcatggtgtcagacatcttggggttgacaacataccagcctgtccggcgtgtcctctcgaggtgggtcaggctgcggtgggaggtcgccggcgcaatgttggtggcgttcacggtgtcggggctaatgcatgaagtgtttttcttctacttaactcgcgcgaggccctcgtgggaggtgacggggttctttgtgttgcatggggtttgcacagccgtggagatggtggtgaagaaggcggtttcaggcaaggtgcggctgcgccgggaggtgtcaggggcgctgacggtggggttcgtgatggtgactggagtgtggttgtttttgccgcagctggtgaggcatggggtagatttgaaaaccattgatgagtatcctgtcatgtttaattatactcagaagaaattgatgggtttgttggggtggtgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggaaatgggaagcattttagagtttcttgataacaaagccattttggtcactggtgctactggctccttagcaaaaatttttgtggagaaggtactgaggagtcaaccgaatgtgaagaaactctatcttcttttgagagcaaccgatgacgagacagctgctctacgcttgcaaaatgaggtttttggaaaagagttgttcaaagttctaaaacaaaatttaggtgcaaatttctattcctttgtatcagaaaaagtgactgtagtacccggtgatattactggtgaagacttgtgtctcaaagacgtcaatttgaaggaagaaatgtggagggaaatcgatgttgttgtcaatctagctgctacaatcaacttcattgaaaggtacgacgtgtctctgcttatcaacacatatggagccaagtatgttttggacttcgcgaagaagtgcaacaaattaaagatatttgttcatgtatctactgcttatgtatctggagagaaaaatgggttaatactggagaagccttattatatgggcgagtcacttaatggaagattaggtctggacattaatgtagagaagaaacttgtggaggcaaaaatcaatgaacttcaagcagcgggggcaacggaaaagtccattaaatcgacaatgaaggacatgggcatcgagagggcaagacactggggatggccaaatgtgtatgtattcaccaaggcattaggggagatgcttttgatgcaatacaaaggggacattccgcttactattattcgtcccaccatcatcaccagcacttttaaagagccctttcctggttgggttgaaggtgtcaggaccatcgataatgtacctgtatattatggtaaagggagattgaggtgtatgctttgcggacccagcacaacaattgacctgataccggcagatatggtcgtgaatgcaacgatagtagccatggtggcgcacgcaaaccaaagatacgtagagccggtgacataccatgtgggatcttcagcggcgaatccaatgaaactgagtgcattaccagagatggcacaccgttacttcaccaagaatccatggatcaacccggatcgcaacccagtacatgtgggtcgggctatggtcttctcctccttctccaccttccacctttatctcacccttaatttcctccttcctttgaaggtactggagatagcaaatacaatattctgccaatggttcaagggtaagtacatggatcttaaaaggaagacgaggttgttgttgcgtttagtagacatttataaaccctacctcttcttccaaggcatctttgatgacatgaacactgagaagttgcggattgctgcaaaagaaaacatggttgaagctgatatgttttactttgatcccagggcaattaactgggaagattacttcttgaaaactcatttcccaggtgtcgtagagcacgttcttaactaagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcg

SEQ ID NO: 29 - DNA, 9742 bp, synthetic construct
  source 1..9742: mol_type = other DNA
gaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcctgtacccccgcgacggcgtgctgaagggcgagacacacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatgaaggccaaaacaatcacaaacccggagatccaagtctccacgaccatgaccaccacgaccacgaccgccactctccccaacttcaagtcctccatcaacttacaccacgtcaagctcggctaccactacttaatctccaatgccctcttcctcgtattcatcccccttttgggcctcgcttcggcccatctctcctccttctcggcccatgacttgtccctgctcttcgacctccttcgccgcaacctcctccctgttgtcgtttgttctttcctcttcgttttattagcaaccctacatttcttgacccggcccaggaatgtctacttggtggactttgcatgctataagcctcacccgaacctgatgacatcccacgagatgttcatggaccggacctcccgggccgggtcgttttctaaggagaatattgagtttcagaggaagatcttggagagggccggtatgggccgggaaacctacgtccccgaatccgtcactaaggtgccgcccgagccgagcatagcagcagccagggccgaggcggaggaggtgatgtacggggcgatcgacgaggtgttggagaagacgggggtgaagccgaagcagataggaatactggtggtgaactgcagcttgtttaacccaacgccgtcgctgtcatccatgatagttaaccattacaagcttaggggtaatatacttagctataatcttggtggcatgggttgcagtgctgggctcatttccattgatcttgccaaggacctcctacaggtttaccgtaacacatatgtgttagtagtgagcacagaaaacatgacccttaattggtactggggcaatgaccgctccatgcttatcaccaactgcctatttcgcatgggtggcgctgccatcatcctctcaaaccgctggcgtgatcgtcgccgatccaagtaccaactccttcacacagtacgcacccacaagggcgctgacgacaagtcctatagatgcgtcttacaacaagaagatgaaaataacaaggtaggtgttgccttatccaaggatctgatggcagttgccggtgaagccctaaaggccaacatcacgacccttggtcccctcgtgctccccatgtcagaacaactcctcttctttgccaccttagtggcacgtaaggtcttcaagatgacgaacgtgaagccatacatcccagatttcaagttggcagcgaagcacttctgcatccatgcaggaggcaaagcagtgttggatgagctcgagacgaacttggagttgacgccatggcaccttgaaccctcgaggatgacactgtataggtttgggaacacatcgagtagctcattatggtacgagttggcatacgctgaagcaaaagggaggatccgtaagggtgatcgaacttggatgattggatttggttcaggtttcaagtgtaacagtgttgtgtggagggctttgaggagtgtcaatccggctagagagaagaatccttggatggatgaaattgagaatttccctgtccatgtgcctaaaatcgcacctatcgcttcgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggtggagaaggagctagagactttcgcagaggtatggatctccgccatagccgccgcctgctactgccgcttcgtccccgccgttgcccctcacggcggcgctctacgcctcctcctcctcctccccgtcgtcctcctcttcattttcctccccctccgcctctcctccttccacctcggcggccccaccgccttgtatctcgtctggcttgccaacttcaagctccttctcttcgcctttcatcttggccctttatctaacccctctctctctctccttcacttcatctccaccaccctcctccccatcaagttcagagatgacccatctaatgatcatgagaaaaacaagagaactctgagttttgagtggcgtaaagttgttctttttgttgctaagttggtgttttttgcgggtattttaaagatttatgagtttagaaaagatttgcctcattttgtgatctcggtgctttactgttttcacttctatctcgggacggagatcaccttagcagcaagcgcagtcatagctcgagccacgctagggttagacctatacccccagttcaacgagccatacttagccacctcgctgcaagacttctgggggcgcaggtggaacctcatggtgtcagacatcttggggttgacaacataccagcctgtccggcgtgtcctctcgaggtgggtcaggctgcggtgggaggtcgccggcgcaatgttggtggcgttcacggtgtcggggctaatgcatgaagtgtttttcttctacttaactcgcgcgaggccctcgtgggaggtgacggggttctttgtgttgcatggggtttgcacagccgtggagatggtggtgaagaaggcggtttcaggcaaggtgcggctgcgccgggaggtgtcaggggcgctgacggtggggttcgtgatggtgactggagtgtggttgtttttgccgcagctggtgaggcatggggtagatttgaaaaccattgatgagtatcctgtcatgtttaattatactcagaagaaattgatgggtttgttggggtggtgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggaaatgggaagcattttagagtttcttgataacaaagccattttggtcactggtgctactggctccttagcaaaaatttttgtggagaaggtactgaggagtcaaccgaatgtgaagaaactctatcttcttttgagagcaaccgatgacgagacagctgctctacgcttgcaaaatgaggtttttggaaaagagttgttcaaagttctaaaacaaaatttaggtgcaaatttctattcctttgtatcagaaaaagtgactgtagtacccggtgatattactggtgaagacttgtgtctcaaagacgtcaatttgaaggaagaaatgtggagggaaatcgatgttgttgtcaatctagctgctacaatcaacttcattgaaaggtacgacgtgtctctgcttatcaacacatatggagccaagtatgttttggacttcgcgaagaagtgcaacaaattaaagatatttgttcatgtatctactgcttatgtatctggagagaaaaatgggttaatactggagaagccttattatatgggcgagtcacttaatggaagattaggtctggacattaatgtagagaagaaacttgtggaggcaaaaatcaatgaacttcaagcagcgggggcaacggaaaagtccattaaatcgacaatgaaggacatgggcatcgagagggcaagacactggggatggccaaatgtgtatgtattcaccaaggcattaggggagatgcttttgatgcaatacaaaggggacattccgcttactattattcgtcccaccatcatcaccagcacttttaaagagccctttcctggttgggttgaaggtgtcaggaccatcgataatgtacctgtatattatggtaaagggagattgaggtgtatgctttgcggacccagcacaacaattgacctgataccggcagatatggtcgtgaatgcaacgatagtagccatggtggcgcacgcaaaccaaagatacgtagagccggtgacataccatgtgggatcttcagcggcgaatccaatgaaactgagtgcattaccagagatggcacaccgttacttcaccaagaatccatggatcaacccggatcgcaacccagtacatgtgggtcgggctatggtcttctcctccttctccaccttccacctttatctcacccttaatttcctccttcctttgaaggtactggagatagcaaatacaatattctgccaatggttcaagggtaagtacatggatcttaaaaggaagacgaggttgttgttgcgtttagtagacatttataaaccctacctcttcttccaaggcatctttgatgacatgaacactgagaagttgcggattgctgcaaaagaaaacatggttgaagctgatatgttttactttgatcccagggcaattaactgggaagattacttcttgaaaactcatttcccaggtgtcgtagagcacgttcttaactaagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggagactactactagtacggagatgaagaagagagaattaaagcatctagggtttgtgaaggtggctgcggtgaacacgatcatgtgtctttcgagtgtttatgagtatgcaaaacagaattctgggccgttgaaatcaacagttggtgcagttgagggtgctgttaccactgtggtttcccctgtctatgagaaattcaagggcgttcctgatgatctccttggtttccttgacgaaaaggtaggtgtggcaagtgaaaaatttgacgagcacgctcctccattggccaagcatttggtatcaaaaactcatgatatcatcgtaaaggcatcccaagtgacccaagaccttatcaaccacgccaagactgatggccctcgcgctgctgcacattacgctgcctctgagtcgaagcagcttgtgtttattcaatccgcgaaaatctggaccaggctcgaccaaatcccgcccttccacatgctagcagacattgctgtccctacagccgctcactggtcagaaaagtacaataaagtcgtggttgacatgagcagtaagggccacgttatcttcagttaccttcccttggttcctatcgatgacattgccaaggcttttaagccggcgggctcagccaaagaggagggtggtgaagatggtgctgcaccggctcatgaagcgcagtcgcagtcggattgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcg

SEQ ID NO: 30 - DNA, 9763 bp, synthetic construct
  source 1..9763: mol_type = other DNA
gaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcctgtacccccgcgacggcgtgctgaagggcgagacacacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatgaaggccaaaacaatcacaaacccggagatccaagtctccacgaccatgaccaccacgaccacgaccgccactctccccaacttcaagtcctccatcaacttacaccacgtcaagctcggctaccactacttaatctccaatgccctcttcctcgtattcatcccccttttgggcctcgcttcggcccatctctcctccttctcggcccatgacttgtccctgctcttcgacctccttcgccgcaacctcctccctgttgtcgtttgttctttcctcttcgttttattagcaaccctacatttcttgacccggcccaggaatgtctacttggtggactttgcatgctataagcctcacccgaacctgatgacatcccacgagatgttcatggaccggacctcccgggccgggtcgttttctaaggagaatattgagtttcagaggaagatcttggagagggccggtatgggccgggaaacctacgtccccgaatccgtcactaaggtgccgcccgagccgagcatagcagcagccagggccgaggcggaggaggtgatgtacggggcgatcgacgaggtgttggagaagacgggggtgaagccgaagcagataggaatactggtggtgaactgcagcttgtttaacccaacgccgtcgctgtcatccatgatagttaaccattacaagcttaggggtaatatacttagctataatcttggtggcatgggttgcagtgctgggctcatttccattgatcttgccaaggacctcctacaggtttaccgtaacacatatgtgttagtagtgagcacagaaaacatgacccttaattggtactggggcaatgaccgctccatgcttatcaccaactgcctatttcgcatgggtggcgctgccatcatcctctcaaaccgctggcgtgatcgtcgccgatccaagtaccaactccttcacacagtacgcacccacaagggcgctgacgacaagtcctatagatgcgtcttacaacaagaagatgaaaataacaaggtaggtgttgccttatccaaggatctgatggcagttgccggtgaagccctaaaggccaacatcacgacccttggtcccctcgtgctccccatgtcagaacaactcctcttctttgccaccttagtggcacgtaaggtcttcaagatgacgaacgtgaagccatacatcccagatttcaagttggcagcgaagcacttctgcatccatgcaggaggcaaagcagtgttggatgagctcgagacgaacttggagttgacgccatggcaccttgaaccctcgaggatgacactgtataggtttgggaacacatcgagtagctcattatggtacgagttggcatacgctgaagcaaaagggaggatccgtaagggtgatcgaacttggatgattggatttggttcaggtttcaagtgtaacagtgttgtgtggagggctttgaggagtgtcaatccggctagagagaagaatccttggatggatgaaattgagaatttccctgtccatgtgcctaaaatcgcacctatcgcttcgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggtggagaaggagctagagactttcgcagaggtatggatctccgccatagccgccgcctgctactgccgcttcgtccccgccgttgcccctcacggcggcgctctacgcctcctcctcctcctccccgtcgtcctcctcttcattttcctccccctccgcctctcctccttccacctcggcggccccaccgccttgtatctcgtctggcttgccaacttcaagctccttctcttcgcctttcatcttggccctttatctaacccctctctctctctccttcacttcatctccaccaccctcctccccatcaagttcagagatgacccatctaatgatcatgagaaaaacaagagaactctgagttttgagtggcgtaaagttgttctttttgttgctaagttggtgttttttgcgggtattttaaagatttatgagtttagaaaagatttgcctcattttgtgatctcggtgctttactgttttcacttctatctcgggacggagatcaccttagcagcaagcgcagtcatagctcgagccacgctagggttagacctatacccccagttcaacgagccatacttagccacctcgctgcaagacttctgggggcgcaggtggaacctcatggtgtcagacatcttggggttgacaacataccagcctgtccggcgtgtcctctcgaggtgggtcaggctgcggtgggaggtcgccggcgcaatgttggtggcgttcacggtgtcggggctaatgcatgaagtgtttttcttctacttaactcgcgcgaggccctcgtgggaggtgacggggttctttgtgttgcatggggtttgcacagccgtggagatggtggtgaagaaggcggtttcaggcaaggtgcggctgcgccgggaggtgtcaggggcgctgacggtggggttcgtgatggtgactggagtgtggttgtttttgccgcagctggtgaggcatggggtagatttgaaaaccattgatgagtatcctgtcatgtttaattatactcagaagaaattgatgggtttgttggggtggtgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggaaatgggaagcattttagagtttcttgataacaaagccattttggtcactggtgctactggctccttagcaaaaatttttgtggagaaggtactgaggagtcaaccgaatgtgaagaaactctatcttcttttgagagcaaccgatgacgagacagctgctctacgcttgcaaaatgaggtttttggaaaagagttgttcaaagttctaaaacaaaatttaggtgcaaatttctattcctttgtatcagaaaaagtgactgtagtacccggtgatattactggtgaagacttgtgtctcaaagacgtcaatttgaaggaagaaatgtggagggaaatcgatgttgttgtcaatctagctgctacaatcaacttcattgaaaggtacgacgtgtctctgcttatcaacacatatggagccaagtatgttttggacttcgcgaagaagtgcaacaaattaaagatatttgttcatgtatctactgcttatgtatctggagagaaaaatgggttaatactggagaagccttattatatgggcgagtcacttaatggaagattaggtctggacattaatgtagagaagaaacttgtggaggcaaaaatcaatgaacttcaagcagcgggggcaacggaaaagtccattaaatcgacaatgaaggacatgggcatcgagagggcaagacactggggatggccaaatgtgtatgtattcaccaaggcattaggggagatgcttttgatgcaatacaaaggggacattccgcttactattattcgtcccaccatcatcaccagcacttttaaagagccctttcctggttgggttgaaggtgtcaggaccatcgataatgtacctgtatattatggtaaagggagattgaggtgtatgctttgcggacccagcacaacaattgacctgataccggcagatatggtcgtgaatgcaacgatagtagccatggtggcgcacgcaaaccaaagatacgtagagccggtgacataccatgtgggatcttcagcggcgaatccaatgaaactgagtgcattaccagagatggcacaccgttacttcaccaagaatccatggatcaacccggatcgcaacccagtacatgtgggtcgggctatggtcttctcctccttctccaccttccacctttatctcacccttaatttcctccttcctttgaaggtactggagatagcaaatacaatattctgccaatggttcaagggtaagtacatggatcttaaaaggaagacgaggttgttgttgcgtttagtagacatttataaaccctacctcttcttccaaggcatctttgatgacatgaacactgagaagttgcggattgctgcaaaagaaaacatggttgaagctgatatgttttactttgatcccagggcaattaactgggaagattacttcttgaaaactcatttcccaggtgtcgtagagcacgttcttaactaagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggagacagagaagaaaaatagcaaggaggtagcgctaaaacacctagcgttcgtgagaattgcaacgatccatattctggcttccgtctcaaatctctacgaatacgctaaacaaaactccggtcctctcaaatccgccgtggaaaaggtcgaaggagccgtcaccaccgtcgtcacccctgtctatcagaaattcaaagatgttcctgattctctcctggtctttctagatcacaaggtgggtgaagtttcgtacaagtttgatgagcatgctcctccaatggctaagaaagtggtgaatcaagcacatgtattgatctacaaggcgacagagaaagctcaaagctttgtgaaagaggctcgtaccggtggtcccaaagctgcgtttaactatgctgcaactgagtacaagtttttcgttgtgaccaactcggttaaagtctgggctaaacttaaccagtataaaccaatccatgcaatgggtgacaaagctttgcctgtggctgcacacttttccagtcggtacaacgatttggtgactgatatgactaatatgggttactctttggttggttatcttccgttggttcctgttgatgacattgttaaggcttatgaaaaggaagatgcaaggagaaagaaaggaggggatacggctggaaagaaaggagagactactgatgcggctgatggtgataaatcatcgtctgattcggagtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcg

SEQ ID NO: 31 - DNA, 9739 bp, synthetic construct
  source 1..9739: mol_type = other DNA
gaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcctgtacccccgcgacggcgtgctgaagggcgagacacacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatgaaggccaaaacaatcacaaacccggagatccaagtctccacgaccatgaccaccacgaccacgaccgccactctccccaacttcaagtcctccatcaacttacaccacgtcaagctcggctaccactacttaatctccaatgccctcttcctcgtattcatcccccttttgggcctcgcttcggcccatctctcctccttctcggcccatgacttgtccctgctcttcgacctccttcgccgcaacctcctccctgttgtcgtttgttctttcctcttcgttttattagcaaccctacatttcttgacccggcccaggaatgtctacttggtggactttgcatgctataagcctcacccgaacctgatgacatcccacgagatgttcatggaccggacctcccgggccgggtcgttttctaaggagaatattgagtttcagaggaagatcttggagagggccggtatgggccgggaaacctacgtccccgaatccgtcactaaggtgccgcccgagccgagcatagcagcagccagggccgaggcggaggaggtgatgtacggggcgatcgacgaggtgttggagaagacgggggtgaagccgaagcagataggaatactggtggtgaactgcagcttgtttaacccaacgccgtcgctgtcatccatgatagttaaccattacaagcttaggggtaatatacttagctataatcttggtggcatgggttgcagtgctgggctcatttccattgatcttgccaaggacctcctacaggtttaccgtaacacatatgtgttagtagtgagcacagaaaacatgacccttaattggtactggggcaatgaccgctccatgcttatcaccaactgcctatttcgcatgggtggcgctgccatcatcctctcaaaccgctggcgtgatcgtcgccgatccaagtaccaactccttcacacagtacgcacccacaagggcgctgacgacaagtcctatagatgcgtcttacaacaagaagatgaaaataacaaggtaggtgttgccttatccaaggatctgatggcagttgccggtgaagccctaaaggccaacatcacgacccttggtcccctcgtgctccccatgtcagaacaactcctcttctttgccaccttagtggcacgtaaggtcttcaagatgacgaacgtgaagccatacatcccagatttcaagttggcagcgaagcacttctgcatccatgcaggaggcaaagcagtgttggatgagctcgagacgaacttggagttgacgccatggcaccttgaaccctcgaggatgacactgtataggtttgggaacacatcgagtagctcattatggtacgagttggcatacgctgaagcaaaagggaggatccgtaagggtgatcgaacttggatgattggatttggttcaggtttcaagtgtaacagtgttgtgtggagggctttgaggagtgtcaatccggctagagagaagaatccttggatggatgaaattgagaatttccctgtccatgtgcctaaaatcgcacctatcgcttcgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggtggagaaggagctagagactttcgcagaggtatggatctccgccatagccgccgcctgctactgccgcttcgtccccgccgttgcccctcacggcggcgctctacgcctcctcctcctcctccccgtcgtcctcctcttcattttcctccccctccgcctctcctccttccacctcggcggccccaccgccttgtatctcgtctggcttgccaacttcaagctccttctcttcgcctttcatcttggccctttatctaacccctctctctctctccttcacttcatctccaccaccctcctccccatcaagttcagagatgacccatctaatgatcatgagaaaaacaagagaactctgagttttgagtggcgtaaagttgttctttttgttgctaagttggtgttttttgcgggtattttaaagatttatgagtttagaaaagatttgcctcattttgtgatctcggtgctttactgttttcacttctatctcgggacggagatcaccttagcagcaagcgcagtcatagctcgagccacgctagggttagacctatacccccagttcaacgagccatacttagccacctcgctgcaagacttctgggggcgcaggtggaacctcatggtgtcagacatcttggggttgacaacataccagcctgtccggcgtgtcctctcgaggtgggtcaggctgcggtgggaggtcgccggcgcaatgttggtggcgttcacggtgtcggggctaatgcatgaagtgtttttcttctacttaactcgcgcgaggccctcgtgggaggtgacggggttctttgtgttgcatggggtttgcacagccgtggagatggtggtgaagaaggcggtttcaggcaaggtgcggctgcgccgggaggtgtcaggggcgctgacggtggggttcgtgatggtgactggagtgtggttgtttttgccgcagctggtgaggcatggggtagatttgaaaaccattgatgagtatcctgtcatgtttaattatactcagaagaaattgatgggtttgttggggtggtgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggaaatgggaagcattttagagtttcttgataacaaagccattttggtcactggtgctactggctccttagcaaaaatttttgtggagaaggtactgaggagtcaaccgaatgtgaagaaactctatcttcttttgagagcaaccgatgacgagacagctgctctacgcttgcaaaatgaggtttttggaaaagagttgttcaaagttctaaaacaaaatttaggtgcaaatttctattcctttgtatcagaaaaagtgactgtagtacccggtgatattactggtgaagacttgtgtctcaaagacgtcaatttgaaggaagaaatgtggagggaaatcgatgttgttgtcaatctagctgctacaatcaacttcattgaaaggtacgacgtgtctctgcttatcaacacatatggagccaagtatgttttggacttcgcgaagaagtgcaacaaattaaagatatttgttcatgtatctactgcttatgtatctggagagaaaaatgggttaatactggagaagccttattatatgggcgagtcacttaatggaagattaggtctggacattaatgtagagaagaaacttgtggaggcaaaaatcaatgaacttcaagcagcgggggcaacggaaaagtccattaaatcgacaatgaaggacatgggcatcgagagggcaagacactggggatggccaaatgtgtatgtattcaccaaggcattaggggagatgcttttgatgcaatacaaaggggacattccgcttactattattcgtcccaccatcatcaccagcacttttaaagagccctttcctggttgggttgaaggtgtcaggaccatcgataatgtacctgtatattatggtaaagggagattgaggtgtatgctttgcggacccagcacaacaattgacctgataccggcagatatggtcgtgaatgcaacgatagtagccatggtggcgcacgcaaaccaaagatacgtagagccggtgacataccatgtgggatcttcagcggcgaatccaatgaaactgagtgcattaccagagatggcacaccgttacttcaccaagaatccatggatcaacccggatcgcaacccagtacatgtgggtcgggctatggtcttctcctccttctccaccttccacctttatctcacccttaatttcctccttcctttgaaggtactggagatagcaaatacaatattctgccaatggttcaagggtaagtacatggatcttaaaaggaagacgaggttgttgttgcgtttagtagacatttataaaccctacctcttcttccaaggcatctttgatgacatgaacactgagaagttgcggattgctgcaaaagaaaacatggttgaagctgatatgttttactttgatcccagggcaattaactgggaagattacttcttgaaaactcatttcccaggtgtcgtagagcacgttcttaactaagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggagacagagaagaaaaatagcaaggaggtagcgctaaaacacctagcgttcgtgagaattgcaacgatccatattctggcttccgtctcaaatctctacgaatacgctaaacaaaactccggtcctctcaaatccgccgtggaaaaggtcgaaggagccgtcaccaccgtcgtcacccctgtctatcagaaattcaaagatgttcctgattctctcctggtctttctagatcacaaggtgggtgaagtttcgtacaagtttgatgagcatgctcctccaatggctaagaaagtggtgaatcaagcacatgtattgatctacaaggcgacagagaaagctcaaagctttgtgaaagaggctcgtaccgatggccctcgcgctgctgcacattacgctgcctctgagtcgaagcagcttgtgtttattcaatccgcgaaaatctggaccaggctcgaccaaatcccgcccttccacatgctagcagacattgctgtccctacagccgctcactggtcagaaaagtacaataaagtcgtggttgacatgagcagtaagggccacgttatcttcagttaccttcccttggttcctatcgatgacattgccaaggcttttaagccggcgggctcagccaaagaggagggtggtgaagatggtgctgcaccggctcatgaagcgcagtcgcagtcggattgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcg

SEQ ID NO: 32 - DNA, 9766 bp, synthetic construct
  source 1..9766: mol_type = other DNA
gaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcctgtacccccgcgacggcgtgctgaagggcgagacacacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatgaaggccaaaacaatcacaaacccggagatccaagtctccacgaccatgaccaccacgaccacgaccgccactctccccaacttcaagtcctccatcaacttacaccacgtcaagctcggctaccactacttaatctccaatgccctcttcctcgtattcatcccccttttgggcctcgcttcggcccatctctcctccttctcggcccatgacttgtccctgctcttcgacctccttcgccgcaacctcctccctgttgtcgtttgttctttcctcttcgttttattagcaaccctacatttcttgacccggcccaggaatgtctacttggtggactttgcatgctataagcctcacccgaacctgatgacatcccacgagatgttcatggaccggacctcccgggccgggtcgttttctaaggagaatattgagtttcagaggaagatcttggagagggccggtatgggccgggaaacctacgtccccgaatccgtcactaaggtgccgcccgagccgagcatagcagcagccagggccgaggcggaggaggtgatgtacggggcgatcgacgaggtgttggagaagacgggggtgaagccgaagcagataggaatactggtggtgaactgcagcttgtttaacccaacgccgtcgctgtcatccatgatagttaaccattacaagcttaggggtaatatacttagctataatcttggtggcatgggttgcagtgctgggctcatttccattgatcttgccaaggacctcctacaggtttaccgtaacacatatgtgttagtagtgagcacagaaaacatgacccttaattggtactggggcaatgaccgctccatgcttatcaccaactgcctatttcgcatgggtggcgctgccatcatcctctcaaaccgctggcgtgatcgtcgccgatccaagtaccaactccttcacacagtacgcacccacaagggcgctgacgacaagtcctatagatgcgtcttacaacaagaagatgaaaataacaaggtaggtgttgccttatccaaggatctgatggcagttgccggtgaagccctaaaggccaacatcacgacccttggtcccctcgtgctccccatgtcagaacaactcctcttctttgccaccttagtggcacgtaaggtcttcaagatgacgaacgtgaagccatacatcccagatttcaagttggcagcgaagcacttctgcatccatgcaggaggcaaagcagtgttggatgagctcgagacgaacttggagttgacgccatggcaccttgaaccctcgaggatgacactgtataggtttgggaacacatcgagtagctcattatggtacgagttggcatacgctgaagcaaaagggaggatccgtaagggtgatcgaacttggatgattggatttggttcaggtttcaagtgtaacagtgttgtgtggagggctttgaggagtgtcaatccggctagagagaagaatccttggatggatgaaattgagaatttccctgtccatgtgcctaaaatcgcacctatcgcttcgtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggtggagaaggagctagagactttcgcagaggtatggatctccgccatagccgccgcctgctactgccgcttcgtccccgccgttgcccctcacggcggcgctctacgcctcctcctcctcctccccgtcgtcctcctcttcattttcctccccctccgcctctcctccttccacctcggcggccccaccgccttgtatctcgtctggcttgccaacttcaagctccttctcttcgcctttcatcttggccctttatctaacccctctctctctctccttcacttcatctccaccaccctcctccccatcaagttcagagatgacccatctaatgatcatgagaaaaacaagagaactctgagttttgagtggcgtaaagttgttctttttgttgctaagttggtgttttttgcgggtattttaaagatttatgagtttagaaaagatttgcctcattttgtgatctcggtgctttactgttttcacttctatctcgggacggagatcaccttagcagcaagcgcagtcatagctcgagccacgctagggttagacctatacccccagttcaacgagccatacttagccacctcgctgcaagacttctgggggcgcaggtggaacctcatggtgtcagacatcttggggttgacaacataccagcctgtccggcgtgtcctctcgaggtgggtcaggctgcggtgggaggtcgccggcgcaatgttggtggcgttcacggtgtcggggctaatgcatgaagtgtttttcttctacttaactcgcgcgaggccctcgtgggaggtgacggggttctttgtgttgcatggggtttgcacagccgtggagatggtggtgaagaaggcggtttcaggcaaggtgcggctgcgccgggaggtgtcaggggcgctgacggtggggttcgtgatggtgactggagtgtggttgtttttgccgcagctggtgaggcatggggtagatttgaaaaccattgatgagtatcctgtcatgtttaattatactcagaagaaattgatgggtttgttggggtggtgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggaggaaatgggaagcattttagagtttcttgataacaaagccattttggtcactggtgctactggctccttagcaaaaatttttgtggagaaggtactgaggagtcaaccgaatgtgaagaaactctatcttcttttgagagcaaccgatgacgagacagctgctctacgcttgcaaaatgaggtttttggaaaagagttgttcaaagttctaaaacaaaatttaggtgcaaatttctattcctttgtatcagaaaaagtgactgtagtacccggtgatattactggtgaagacttgtgtctcaaagacgtcaatttgaaggaagaaatgtggagggaaatcgatgttgttgtcaatctagctgctacaatcaacttcattgaaaggtacgacgtgtctctgcttatcaacacatatggagccaagtatgttttggacttcgcgaagaagtgcaacaaattaaagatatttgttcatgtatctactgcttatgtatctggagagaaaaatgggttaatactggagaagccttattatatgggcgagtcacttaatggaagattaggtctggacattaatgtagagaagaaacttgtggaggcaaaaatcaatgaacttcaagcagcgggggcaacggaaaagtccattaaatcgacaatgaaggacatgggcatcgagagggcaagacactggggatggccaaatgtgtatgtattcaccaaggcattaggggagatgcttttgatgcaatacaaaggggacattccgcttactattattcgtcccaccatcatcaccagcacttttaaagagccctttcctggttgggttgaaggtgtcaggaccatcgataatgtacctgtatattatggtaaagggagattgaggtgtatgctttgcggacccagcacaacaattgacctgataccggcagatatggtcgtgaatgcaacgatagtagccatggtggcgcacgcaaaccaaagatacgtagagccggtgacataccatgtgggatcttcagcggcgaatccaatgaaactgagtgcattaccagagatggcacaccgttacttcaccaagaatccatggatcaacccggatcgcaacccagtacatgtgggtcgggctatggtcttctcctccttctccaccttccacctttatctcacccttaatttcctccttcctttgaaggtactggagatagcaaatacaatattctgccaatggttcaagggtaagtacatggatcttaaaaggaagacgaggttgttgttgcgtttagtagacatttataaaccctacctcttcttccaaggcatctttgatgacatgaacactgagaagttgcggattgctgcaaaagaaaacatggttgaagctgatatgttttactttgatcccagggcaattaactgggaagattacttcttgaaaactcatttcccaggtgtcgtagagcacgttcttaactaagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacaatggagactactactagtacggagatgaagaagagagaattaaagcatctagggtttgtggaggtggctgcggtgaacacgatcatgtgtctttcgagtgtttatgagtatgcaaaacagaattctgggccgttgaaatcaacagttggtgcagttgagggtgctgttaccactgtggtttcccctgtctatgagaaattcaagggcgttcctgatgatctccttggtttccttgacgaaaaggtaggtgtggcaagtgaaaaatttgacgagcacgctcctccattggccaagcatttggtatcaaaaactcatgatatcatcgtaaaggcatcccaagtgacccaagaccttatcaaccacgccaagactggtggtcccaaagctgcgtttaactatgctgcaactgagtacaagtttttcgttgtgaccaactcggttaaagtctgggctaaacttaaccagtataaaccaatccatgcaatgggtgacaaagctttgcctgtggctgcacacttttccagtcggtacaacgatttggtgactgatatgactaatatgggttactctttggttggttatcttccgttggttcctgttgatgacattgttaaggcttatgaaaaggaagatgcaaggagaaagaaaggaggggatacggctggaaagaaaggagagactactgatgcggctgatggtgataaatcatcgtctgattcggagtaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcg

SEQ ID NO: 33 - DNA, 756 bp, unidentified
  source 1..756: mol_type = genomic DNA
atggacgaggcagctgccaagccacctactgatgcagtagcagaggatgagaggaaactcaaatatttggatttcgttcaagttacagcgatctatgtgatagtttgcttctccactttgtacgaatacgccaaaggaaactccgggccactgaaacctggtgttaaagctgtagaagccactgttaaaaccgtcatcggaccggtttaccataaattccacgacgtgcctttcgatctcctcaagttcgtcgacttcaaggttgaagatttgatgatcgaagtagatcgccatgtaccttatttactgaagcagacgtcatgccaagcattgttgctggctcagaaggctcctgaattagctcgagatctcgccggcgagatacagcgtgatggcttggttgacacggcgaacagcgtagccaaagcgctttacaaaaagtacgaacctacagccaaggagctatacgaaaagtacgagccattagccgagaagaatgcggtttcggcttggcggtctttgaacaggcttccgctattccctcaagtggctcagattttggtgccgacggctgcttattggagcgagaaatacaatcaagctgtggcgtatgcgtcggagagaagctatgtggcagcgtattattttccgataattcctctggagagaatcgcgaaggtgtttgaaggcggcggcggcgctgagaacgggcattccgtttccgtgaatgacggctcggttactttggcgccatga

SEQ ID NO: 34 - protein, 251 aa, unidentified
MDEAAAKPPTDAVAEDERKLKYLDFVQVTAIYVIVCFSTLYEYAKGNSGPLKPGVKAVEATVKTVIGPVYHKFHDVPFDLLKFVDFKVEDLMIEVDRHVPYLLKQTSCQALLLAQKAPELARDLAGEIQRDGLVDTANSVAKALYKKYEPTAKELYEKYEPLAEKNAVSAWRSLNRLPLFPQVAQILVPTAAYWSEKYNQAVAYASERSYVAAYYFPIIPLERIAKVFEGGGGAENGHSVSVNDGSVTLAP

SEQ ID NO: 35 - DNA, 31 bp, synthetic construct
  source 1..31: mol_type = other DNA
gggcccggcgcgccaatggaggtggagaagg

SEQ ID NO: 36 - DNA, 30 bp, synthetic construct
  source 1..30: mol_type = other DNA
gggcccttaattaatcaccaccccaacaaa

SEQ ID NO: 37 - DNA, 33 bp, synthetic construct
  source 1..33: mol_type = other DNA
gggcccggcgcgccaatggaggaaatgggaagc

SEQ ID NO: 38 - DNA, 35 bp, synthetic construct
  source 1..35: mol_type = other DNA
gggcccttaattaattagttaagaacgtgctctac

SEQ ID NO: 39 - DNA, 35 bp, synthetic construct
  source 1..35: mol_type = other DNA
gggcccggcgcgccaatgaaggccaaaacaatcac

SEQ ID NO: 40 - DNA, 32 bp, synthetic construct
  source 1..32: mol_type = other DNA
gggcccttaattaactacgaagcgataggtgc

SEQ ID NO: 41 - DNA, 39 bp, synthetic construct
  source 1..39: mol_type = other DNA
gggcccggcgcgccaatggaagtcaacaatttctactcc

SEQ ID NO: 42 - DNA, 33 bp, synthetic construct
  source 1..33: mol_type = other DNA
gggcccttaattaatcaacctcccatacacacc

SEQ ID NO: 43 - DNA, 35 bp, synthetic construct
  source 1..35: mol_type = other DNA
gggcccggcgcgccaatggagaattcacaatcatc

SEQ ID NO: 44 - DNA, 30 bp, synthetic construct
  source 1..30: mol_type = other DNA
gggcccttaattaatcagccgtaccagtag

SEQ ID NO: 45 - DNA, 34 bp, synthetic construct
  source 1..34: mol_type = other DNA
gggcccggcgcgccaatggacacccaacttgaac

SEQ ID NO: 46 - DNA, 34 bp, synthetic construct
  source 1..34: mol_type = other DNA
gggcccttaattaatcattgcacagcaacagcag

SEQ ID NO: 47 - DNA, 36 bp, synthetic construct
  source 1..36: mol_type = other DNA
gggcccggcgcgccaatggagaaaatggaggactcg

SEQ ID NO: 48 - DNA, 32 bp, synthetic construct
  source 1..32: mol_type = other DNA
gggcccttaattaatcaatcgtgatgctggcg

SEQ ID NO: 49 - DNA, 35 bp, synthetic construct
  source 1..35: mol_type = other DNA
gggcccggcgcgccaatggcagatcaaactagggg

SEQ ID NO: 50 - DNA, 35 bp, synthetic construct
  source 1..35: mol_type = other DNA
gggcccttaattaattaagaggtggttgtcgtctg

SEQ ID NO: 51 - DNA, 40 bp, synthetic construct
  source 1..40: mol_type = other DNA
gggcccggcgcgccaatgaaacttgttatgcatgcacacc

SEQ ID NO: 52 - DNA, 34 bp, synthetic construct
  source 1..34: mol_type = other DNA
gggcccttaattaatcaggtccggcctgtagtcc

SEQ ID NO: 53 - DNA, 31 bp, synthetic construct
  source 1..31: mol_type = other DNA
gggcccggcgcgccaatggctgactaccgtg

SEQ ID NO: 54 - DNA, 31 bp, synthetic construct
  source 1..31: mol_type = other DNA
gggcccttaattaatcaagtcctggtagtcc

SEQ ID NO: 55 - DNA, 39 bp, synthetic construct
  source 1..39: mol_type = other DNA
gggcccggcgcgccaatggagactactactagtacggag

SEQ ID NO: 56 - DNA, 32 bp, synthetic construct
  source 1..32: mol_type = other DNA
gggcccttaattaatcaatccgactgcgactg

SEQ ID NO: 57 - DNA, 29 bp, synthetic construct
  source 1..29: mol_type = other DNA
gggcccttaattaaatccgactgcgactg

SEQ ID NO: 58 - DNA, 38 bp, synthetic construct
  source 1..38: mol_type = other DNA
gggcccggcgcgccaatggagacagagaagaaaaatag

SEQ ID NO: 59 - DNA, 35 bp, synthetic construct
  source 1..35: mol_type = other DNA
agcgcgagggccatcggtacgagcctctttcacaa

SEQ ID NO: 60 - DNA, 35 bp, synthetic construct
  source 1..35: mol_type = other DNA
ttgtgaaagaggctcgtaccgatggccctcgcgct

SEQ ID NO: 61 - DNA, 34 bp, synthetic construct
  source 1..34: mol_type = other DNA
gggcccttaattaactactccgaatcagacgatg

SEQ ID NO: 62 - DNA, 38 bp, synthetic construct
  source 1..38: mol_type = other DNA
gcagctttgggaccaccagtcttggcgtggttgataag

SEQ ID NO: 63 - DNA, 37 bp, synthetic construct
  source 1..37: mol_type = other DNA
ttatcaaccacgccaagactggtggtcccaaagctgc

SEQ ID NO: 64 - DNA, 40 bp, synthetic construct
  source 1..40: mol_type = other DNA
catgtggaagggcgggatatactggttaagtttagcccag

SEQ ID NO: 65 - DNA, 42 bp, synthetic construct
  source 1..42: mol_type = other DNA
gtctgggctaaacttaaccagtatatcccgcccttccacatg

SEQ ID NO: 66 - DNA, 36 bp, synthetic construct
  source 1..36: mol_type = other DNA
cccattgcatggattggtttttggtcgagcctggtc

SEQ ID NO: 67 - DNA, 36 bp, synthetic construct
  source 1..36: mol_type = other DNA
ggaccaggctcgaccaaaaaccaatccatgcaatgg

SEQ ID NO: 68 - DNA, 45 bp, synthetic construct
  source 1..45: mol_type = other DNA
gtcatcgataggaaccaagggaagataaccaaccaaagagtaacc

SEQ ID NO: 69 - DNA, 44 bp, synthetic construct
  source 1..44: mol_type = other DNA
ggttactctttggttggttatcttcccttggttcctatcgatga

SEQ ID NO: 70 - DNA, 42 bp, synthetic construct
  source 1..42: mol_type = other DNA
tcatcaacaggaaccaacggaaggtaactgaagataacgtgg

SEQ ID NO: 71 - DNA, 42 bp, synthetic construct
  source 1..42: mol_type = other DNA
gccacgttatcttcagttaccttccgttggttcctgttgatg

SEQ ID NO: 72 - DNA, 28 bp, synthetic construct
  source 1..28: mol_type = other DNA
tatagtcgacatggaggtggagaaggag

SEQ ID NO: 73 - DNA, 28 bp, synthetic construct
  source 1..28: mol_type = other DNA
tataggatcctcaccaccccaacaaacc

SEQ ID NO: 74 - DNA, 34 bp, synthetic construct
  source 1..34: mol_type = other DNA
tatagtcgacatggagactactactagtacggag

SEQ ID NO: 75 - DNA, 29 bp, synthetic construct
  source 1..29: mol_type = other DNA
tataggatcctcaatccgactgcgactgc

SEQ ID NO: 76 - DNA, 37 bp, synthetic construct
  source 1..37: mol_type = other DNA
tatagtcgacatggagactactactagtacggagatg

SEQ ID NO: 77 - DNA, 37 bp, synthetic construct
  source 1..37: mol_type = other DNA
tataggatccttatttgtatagttctaccatgccatg

SEQ ID NO: 78 - DNA, 20 bp, synthetic construct
  source 1..20: mol_type = other DNA
actgcactgtgattgatgcc

SEQ ID NO: 79 - DNA, 20 bp, synthetic construct
  source 1..20: mol_type = other DNA
gacaccagtttccacacgac

SEQ ID NO: 80 - DNA, 25 bp, synthetic construct
  source 1..25: mol_type = other DNA
atggaacgagctatacaaggaaacg

SEQ ID NO: 81 - DNA, 24 bp, synthetic construct
  source 1..24: mol_type = other DNA
cagggcatcctcttgattcattac

SEQ ID NO: 82 - DNA, 20 bp, synthetic construct
  source 1..20: mol_type = other DNA
aggagctagagaccttcgca

SEQ ID NO: 83 - DNA, 20 bp, synthetic construct
  source 1..20: mol_type = other DNA
gcaacacaaagaaccccgtc

SEQ ID NO: 84 - DNA, 20 bp, synthetic construct
  source 1..20: mol_type = other DNA
cgtagagccggtgacatacc

SEQ ID NO: 85 - DNA, 20 bp, synthetic construct
  source 1..20: mol_type = other DNA
aacgtgctctacgacacctg

SEQ ID NO: 86 - DNA, 20 bp, synthetic construct
  source 1..20: mol_type = other DNA
tacatttcttgacccggccc

SEQ ID NO: 87 - DNA, 20 bp, synthetic construct
  source 1..20: mol_type = other DNA
ggtgcgtactgtgtgaagga

SEQ ID NO: 88 - DNA, 20 bp, synthetic construct
  source 1..20: mol_type = other DNA
atatggtgagcaagggcgag

SEQ ID NO: 89 - DNA, 20 bp, synthetic construct
  source 1..20: mol_type = other DNA
cggatcttgaagtgggcctt

SEQ ID NO: 90 - DNA, 20 bp, synthetic construct
  source 1..20: mol_type = other DNA
gcatcccaagtgacccaaga

SEQ ID NO: 91 - DNA, 20 bp, synthetic construct
  source 1..20: mol_type = other DNA
ggctgtagggacagcaatgt

SEQ ID NO: 92 - DNA, 20 bp, synthetic construct
  source 1..20: mol_type = other DNA
gctgcacacttttccagtcg

SEQ ID NO: 93 - DNA, 20 bp, synthetic construct
  source 1..20: mol_type = other DNA
ccatcagccgcatcagtagt

SEQ ID NO: 94 - DNA, 20 bp, synthetic construct
  source 1..20: mol_type = other DNA
ttctttgcctgcctaacagc

SEQ ID NO: 95 - DNA, 21 bp, synthetic construct
  source 1..21: mol_type = other DNA
ttgttaccagccatattgccc

SEQ ID NO: 96 - DNA, 1647 bp, unidentified
  source 1..1647: mol_type = genomic DNA
atggcctcagcagcagtagccaactatgaagaagagattgttcgccccgtcgccgacttctcccctagtctctggggtgatcagttcctttcattctccattgacaatcaggttgcacaaatgtatgctcaagagattgaagcactgaaggaacaaacgaggagtatgctgttagcaaccggaaggaaattggctgatacattgaatttgattgacattattgaacgccttggcatatcctaccactttgagaaagaaattgatgatcttttggatcatattttcaaccaaaactcaaacttcgatgatttcaccacttctgcacttcaatttcgattgcttaggcaacatggtttcaacatctctcctgaaattttcagcaaattccaagatgaaaacggcaaattcaaggagtctcttgccagtgatgtcttaggattgttgaacttgtatgaagcttcacatgtaagaactcatgctgacgatatcttggaagacgcacgtgctttctccactatccatcttgaatccgcaactccacatttgaattctccactaaaggagcaagtgacacatgcccttgagcgatgtttgcacaagggcgttcctagagtcgagacccgattcttcatctcttcaatctatgagaaggaaaaatcgatgaataatatgttacttcgatttgccaaattggattttaacttgctccagatgttgcacaaacaagaacttgctgaagtatcaaggtggtggaaaggtttggatttcgtaacaacactttcatatgctagagatcgagtagttgaatgctacttttgggcattaggagtttattttgagcctcaatactctcaagctcgtgtcatgctcgttaagacaatatcaatgatttcgattgtcgatgacacctttgatgcttatggtacagttaaagaactcgaggcatacacaaatgccatacaaagatggcatatcaaggaaattgatcggcttcctgattacatgaaaatcagctataaagctattctagatctttacaacgattatgaaaaggaattgtctagtgctggaagatctcatattgtcagccatgcaattcaaagaatgaaagaagtagtaagaaattacaatgtcgagtcaacatggtttattgaaggatataagccacctgtttctaaatacctaagcaatgcactggcaactactacatattactaccttgcgacaacatcatatttgggtattaagtctgttacggagcaggattttgagtggttatcaaagaatcctaaaattcttgaagctagtgtgattatatgccgagttattgatgacacagccacgtacgaggttgagaaaagcaggggacaaattgcaacaagaattgagtgttgcatgagagattatggtctatcaacaaaagaggcaatggataaattccaaaaaatggctgagacagcatggaaggatcttaatgaaggtcttcttaggcccactcccgtatctgcagagtttttaaatcctattctcaatcttgctcgtattgttgaggtgacatatatacacaatctagatggatatactcatccggagaaagtcttaaaacctcacattgttgccctacttgtggactccatccaaatttga

SEQ ID NO: 97 - protein, 548 aa, unidentified
MASAAVANYEEEIVRPVADFSPSLWGDQFLSFSIDNQVAQMYAQEIEALKEQTRSMLLATGRKLADTLNLIDIIERLGISYHFEKEIDDLLDHIFNQNSNFDDFTTSALQFRLLRQHGFNISPEIFSKFQDENGKFKESLASDVLGLLNLYEASHVRTHADDILEDARAFSTIHLESATPHLNSPLKEQVTHALERCLHKGVPRVETRFFISSIYEKEKSMNNMLLRFAKLDFNLLQMLHKQELAEVSRWWKGLDFVTTLSYARDRVVECYFWALGVYFEPQYSQARVMLVKTISMISIVDDTFDAYGTVKELEAYTNAIQRWHIKEIDRLPDYMKISYKAILDLYNDYEKELSSAGRSHIVSHAIQRMKEVVRNYNVESTWFIEGYKPPVSKYLSNALATTTYYYLATTSYLGIKSVTEQDFEWLSKNPKILEASVIICRVIDDTATYEVEKSRGQIATRIECCMRDYGLSTKEAMDKFQKMAETAWKDLNEGLLRPTPVSAEFLNPILNLARIVEVTYIHNLDGYTHPEKVLKPHIVALLVDSIQI

SEQ ID NO: 98 - DNA, 1104 bp, unidentified
  source 1..1104: mol_type = genomic DNA
atgcataaatttactggtgtcaatgccaagtttcagcaacccgcgttgaggaacctcagccccgtggtggttgagagggagagggaggagttcgtggggttcttcccgcagatcgtccgcgatctgaccgaggacggcatcggacacccggaggtgggcgacgctgtggcgcggctgaaggaggtgctgcaatacaacgctcccggtgggaaatgcaaccgtgggctgacggtggtggctgcgtaccgggagctgtcggggccggggcagaaggatgctgagagcctgcggtgcgcgctggccgtgggttggtgcatcgagttgttccaggccttcttcctggtggctgatgatatcatggatcagtccctcacgcgccgggggcagctgtgttggtataagaaggagggggtcggtttggatgccatcaacgactccttcctcctcgaatcctctgtgtacagagtgctgaagaagtactgcaggcagcggccgtattacgtgcatctgttggagctcttcctgcagaccgcctaccagactgagctcgggcagatgctggacctcatcacagctcccgtctccaaagtggatttgagtcacttcagtgaggagaggtacaaagccatcgttaagtacaagactgccttctactccttctacctacccgtggctgctgccatgtatatggttgggatcgacagtaaggaagaacacgagaatgccaaagccatcctgctggagatgggggaatacttccagatccaggatgattacctggactgctttggggacccggcgctcacggggaaggtgggcaccgacatccaggacaataaatgcagctggctcgtggtgcagtgcctgcagcgcgtcacgccggagcagcggcagctcctggaggacaactacggccgtaaggagcccgagaaggtggcgaaggtgaaggagctgtatgaggccgtggggatgagggctgcgttccagcagtacgaggagagcagctaccggcgcctgcaggaactgatagagaagcactcgaaccgcctcccgaaggagatcttcctcggcctggcacagaagatctacaaacgccagaaatga

SEQ ID NO: 99 - protein, 367 aa, unidentified
MHKFTGVNAKFQQPALRNLSPVVVEREREEFVGFFPQIVRDLTEDGIGHPEVGDAVARLKEVLQYNAPGGKCNRGLTVVAAYRELSGPGQKDAESLRCALAVGWCIELFQAFFLVADDIMDQSLTRRGQLCWYKKEGVGLDAINDSFLLESSVYRVLKKYCRQRPYYVHLLELFLQTAYQTELGQMLDLITAPVSKVDLSHFSEERYKAIVKYKTAFYSFYLPVAAAMYMVGIDSKEEHENAKAILLEMGEYFQIQDDYLDCFGDPALTGKVGTDIQDNKCSWLVVQCLQRVTPEQRQLLEDNYGRKEPEKVAKVKELYEAVGMRAAFQQYEESSYRRLQELIEKHSNRLPKEIFLGLAQKIYKRQK

SEQ ID NO: 100 - DNA, 1167 bp, unidentified
  source 1..1167: mol_type = genomic DNA
atgaagaccctcatcgccgcctactccggggtcctgcggggtgagcgtcgggcggaagctgcccgcagcgaaaacaagaataaaggatctgccctgtcacgcgaggggtctgggcgatggggcactggctccagcatcctctcagccctccaagacatcttctctgtcacctggctcaacagatctaaggtggaaaaacagctgcaggtcatctcagtactacaatgggtcctatccttcctggtgctaggagtggcctgcagtgtcatcctcatgtacaccttctgcacagactgctggctgatagctgtgctctacttcacctggctggcatttgactggaacacgcccaagaaaggtggcaggagatcgcagtgggtgcgaaactgggccgtgtggcgctacttccgagactactttcccatccagctggtgaagacacacaacctgctgaccaccaggaactatatctttggataccacccccatggcatcatgggcctgggtgccttctgtaacttcagcacagaggctactgaagtcagcaagaagtttcctggcataaggccctatttggctacgttggctggtaacttccggatgcctgtgcttcgcgagtacctgatgtctggaggcatctgccctgtcaaccgagacaccatagactacttgctctccaagaatgggagtggcaatgctatcatcatcgtggtgggaggtgcagctgagtccctgagctccatgcctggcaagaacgcagtcaccctgaagaaccgcaaaggctttgtgaagctggccctgcgccatggagctgatctggttcccacttattcctttggagagaatgaggtatacaagcaggtgatctttgaggagggttcctggggccgatgggtccagaagaagttccagaagtatattggtttcgccccctgcatcttccatggccgaggcctcttctcctctgacacctgggggctggtgccctactccaagcccatcaccaccgtcgtgggggagcccatcactgtccccaagctggagcacccgacccagaaagacatcgacctgtaccatgccatgtacatggaggccctggtgaagctctttgacaatcacaagaccaaatttggccttccagagactgaggtgctggaggtgaactga

SEQ ID NO: 101 - protein, 388 aa, unidentified
MKTLIAAYSGVLRGERRAEAARSENKNKGSALSREGSGRWGTGSSILSALQDIFSVTWLNRSKVEKQLQVISVLQWVLSFLVLGVACSVILMYTFCTDCWLIAVLYFTWLAFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVNRDTIDYLLSKNGSGNAIIIVVGGAAESLSSMPGKNAVTLKNRKGFVKLALRHGADLVPTYSFGENEVYKQVIFEEGSWGRWVQKKFQKYIGFAPCIFHGRGLFSSDTWGLVPYSKPITTVVGEPITVPKLEHPTQKDIDLYHAMYMEALVKLFDNHKTKFGLPETEVLEVN

SEQ ID NO: 102 - DNA, 789 bp, unidentified
  source 1..789: mol_type = genomic DNA
atggatcacctggagcgctgcgcgtggtttctccgcgggacgctggtgagggcgacggtgcggcgccacctgccctgggcgctggtggccgccatgctggcgggctcggtcgtcaaggagctgtccccgctgcccgagagctacctcagcaacaagcgcaacgtcctcaacgtgtattttgttaaattggcctgggcctggacagtctgtctcctcctgcctttcattgcccttaccaactaccacctgacgggcaagaccagcctggtcctgcggcggttgagcaccctcctcgtgggcacagccatctggtatatctgcacagccctcttctccaacattgagcactatacaggcagctgctaccagtccccggccctggagggcgtcagacaggagcatcgcagcaagcagcagtgccaccgggaagggggcttctggcatggctttgacatctcgggccactccttcctcttgaccttctgtgcgctcatgattgtggaagagatggccgtgctgcatgaggtgaagacggaccgcggccaccacctccatgccgccatcaccaccctggttgtcgccctgggtttcctgaccttcatctgggtgtggatgtttctgtgcacagccgtttatttccacgacttgacccagaaagtgttcggcaccatgtttggtttgttgggctggtacggaacatatgggtattggtatctgaagtctttctctccaggacttcctccccagagttgtagtttgactttgaagcgagatacttacaagaaatga

SEQ ID NO: 103 - protein, 262 aa, unidentified
MDHLERCAWFLRGTLVRATVRRHLPWALVAAMLAGSVVKELSPLPESYLSNKRNVLNVYFVKLAWAWTVCLLLPFIALTNYHLTGKTSLVLRRLSTLLVGTAIWYICTALFSNIEHYTGSCYQSPALEGVRQEHRSKQQCHREGGFWHGFDISGHSFLLTFCALMIVEEMAVLHEVKTDRGHHLHAAITTLVVALGFLTFIWVWMFLCTAVYFHDLTQKVFGTMFGLLGWYGTYGYWYLKSFSPGLPPQSCSLTLKRDTYKK

SEQ ID NO: 104 - DNA, 681 bp, unidentified
  source 1..681: mol_type = genomic DNA
atggccgaatcaaaccctaagcctcaactacccgagatggctcagacggaggaagagaaattgaagtatttggaatttttgcaagtggcgatgattcatgcggcgctctatgtagcgaaggtgtatggttacgccaaagagaattctggtcccctgaagcctggtgttcagaccgtcgaaggcaccgtcaagaccgtggttggccctgtctatgataaatttcatgatgtccctgttgaggtcctcaagtttgttgaccgcaaggttgatgagtctgttcgtaagattgagactcgtgttcctcctatggtcaagcaggctcctgcagcagctcgctccgttgcagccgatgtcaaaagtgctggtgtgataggagctgcatcaggacttgcaaaaacagtctatgccaagtatgagcctgctgccaagggactttacaccaagtacgagccaatggccgagcaatatgcagcatcagcttggctctctctcaaccgacttcccgttgttcctaaagttactcaagcagttggtccaacagctgcttactattctggaaagtacaatgagattgttcagcaatctgcagagaaagggtacaaggttgcatcatatcttccattggtaccgactgaaaagatttctaaggtgttcagcactcagcctctgacttccaactaa

SEQ ID NO: 105 - protein, 226 aa, unidentified
MAESNPKPQLPEMAQTEEEKLKYLEFLQVAMIHAALYVAKVYGYAKENSGPLKPGVQTVEGTVKTVVGPVYDKFHDVPVEVLKFVDRKVDESVRKIETRVPPMVKQAPAAARSVAADVKSAGVIGAASGLAKTVYAKYEPAAKGLYTKYEPMAEQYAASAWLSLNRLPVVPKVTQAVGPTAAYYSGKYNEIVQQSAEKGYKVASYLPLVPTEKISKVFSTQPLTSN

SEQ ID NO: 106 - DNA, 2598 bp, Gossypium hirsutum
  source 1..2598: mol_type = genomic DNA
atggaagtggcggtgatcggaggtgggataaaagggttggtttcggcctacgtactggtcaaagccggcgtggacgtggtggtttacgagaaagaagagcaattaggcggccatgcgaagactgttaacttcgacgccgttgacttagaccttggcttcttgtttcttaatcctgcaagatatgcaacactgttggatataatcgacagccttggtgttgatgtagaaacatccgatgtttcattctctataagccatgacaaaggcaacaatggctatgaatggtgcagtcaatatggattttccaattactttgcacaaaagaagaaactgttgaaccctttcaattggcaaaaccttagagagatcatcagattcagcaacgatgtcgaaagttaccttggatcacttgagaacaacccagacattgatcgtactgagaccttgggacagttcataaaatccaagggccactctgaaaattttcaaaacacttacctggctcctatatgtggttcaatgtggtcaagctccaaggaagatgttatgagcttttcagcattttccatcctttcattttgccgtactcatcatttgtaccagcaatttgggcagccacagtggttgactatcaaagggcactcacattttgttaaaagggttagggaagtgctggagactaaaggttgtcaatttaaactcggttgtgaagtacaatctgttttgcctgctgataatggtaccaccatggtctgtggagatggtttccaagaaacttacaatggatgcataatggctgttgatgctcccactgccctaaaattattaggaaaccaagcaacatttgaagaaacaagagtactgggtgctttccaatatgctaccagtgatattttccttcaccgggatagtactttaatgccacaaaacaaatcagcttggagtgcattgaattttctcaatagtagcaaaaataatgcattcttaacatactggctcaatgcattacagaatattgggaaaacaagtgagccattttttgtgactgtcaatccagaccataccccgaagaataccttgcttaagtggtcgactggccatgcaattccctctgttgctgcatcaaaagcttcacttgagcttggtcagattcaggggaagagaggaatctggttctgtggctatgacttcaatcaggatgaactaaaggctggtatggatgctgcacatggtatcttgggaaagcattcttctgttctgcatagtccaaagagtatgtcaccctctttcatggaaacaacggcacgcctctttgttactaaattctttcaacaatatatatctatgggctgtgtaactttcttagaggaaggaggcagaattttcactttcaaaggaaacatggaaaagtgtcctcttaaaacagttctgaaagtgcataatcctcagttttactggaggatcatgaaagaagctgatataggccttgcagatgcatatatccatggagatttttcttttcttgatgaaactgaaggccttcttaatcttttccggattcttgttgccaataaagagaactcagctgcctcaggatcgaataaaagaaggacttggtggtcacctgctctgttaacagctagtatatcatctgcaaagtattttgtgaagcatctcttgagacaaaatactattacacaagctcgtaggaacatttctcgtcattatgatctgagtaatgaacttttcactctatacttgggcaaaatgatgcaatactcttctggagtctttaggacgggagaagaacatttggacgttgcacagcgtagaaaaatcagttctctaattgagaaagcaaggatagagaaacggcacgaagttctcgacattgggtgcggttggggaagcttagctattgaaactgtgaaaagaacaggatgcaaatatactggcatcactctatcagaacagcaactgaaatatgctcaagaaaaagtgaaggaagctggactccaggataacatcaaaatacttctctgtgactatcgccagttacctaaggaacaccaatttgacagaatcatatctgtagagatggtagaacatgttggtgaagaatatattgaggagttttacagatgctgtgaccaattactgaaagaagatgggctttttgttcttcagttcatatctatcccagaagagctttccaaagaaatccagcaaacagcaggttttctaaaggaatatatattccctggaggaaccctgctttctttggataggaatttatcagccatggctgctgcaacaagattcagtgtggagcatgtggaaaatataggaatgagttattaccacacactgagatggtggagaaaacttttcctggaaaacacaagcaaagttctagctctggggttcgacgagaagttcatgaggacatgggaatactatttcgattactgcgctgccggttttaagacaggaactcttatagattaccaggttgtattttcaagggccggaaatttcggtacactcggagatccatacaaaggtttcccttctgcatattccttcatggatgattga

SEQ ID NO: 107 - protein, 865 aa, Gossypium hirsutum
MEVAVIGGGIKGLVSAYVLVKAGVDVVVYEKEEQLGGHAKTVNFDAVDLDLGFLFLNPARYATLLDIIDSLGVDVETSDVSFSISHDKGNNGYEWCSQYGFSNYFAQKKKLLNPFNWQNLREIIRFSNDVESYLGSLENNPDIDRTETLGQFIKSKGYSENFQNTYLAPICGSMWSSSKEDVMSFSAFSILSFCRTHHLYQQFGQPQWLTIKGHSHFVKRVREVLETKGCQFKLGCEVQSVLPADNGTTMVCGDGFQETYNGCIMAVDAPTALKLLGNQATFEETRVLGAFQYATSDIFLHRDSTLMPQNKSAWSALNFLNSSKNNAFLTYWLNALQNIGKTSEPFFVTVNPDHTPKNTLLKWSTGHAIPSVAASKASLELGQIQGKRGIWFCGYDFNQDELKAGMDAAHGILGKHSSVLHSPKSMSPSFMETTARLFVTKFFQQYISMGCVIFLEEGGRIFTFKGNMEKCPLKTVLKVHNPQFYWRIMKEADIGLADAYIHGDFSFLDETEGLLNLFRILVANKENSAASGSNKRRTWWSPALLTASISSAKYFVKHLLRQNTITQARRNISRHYDLSNELFTLYLGKMMQYSSGVFRTGEEHLDVAQRRKISSLIEKARIEKRHEVLDIGCGWGSLAIETVKRTGCKYTGITLSEQQLKYAQEKVKEAGLQDNIKILLCDYRQLPKEHQFDRIISVEMVEHVGEEYIEEFYRCCDQLLKEDGLFVLQFISIPEELSKEIQQTAGFLKEYIFPGGTLLSLDRNLSAMAAATRFSVEHVENIGMSYYHTLRWWRKLFLENTSKVLALGFDEKFMRTWEYYFDYCAAGFKTGTLIDYQVVFSRAGNFGTLGDPYKGFPSAYSFMDD

SEQ ID NO: 108 - DNA, 696 bp, Gossypium hirsutum
  source 1..696: mol_type = genomic DNA
atggcaaccatggagaaggggttagagtttgagaacaagaatttaggcctcaagcacctaaacttcgtcagggtggctacaatccacgccttggtttgcgtctcgaacctttacgattactctaaacagaactcggggccgttgagatccaccgttgacgccgtcgagcatgccgttaccactgccgtcggtcccgcctacgggaaatttaaggatgttcctgatcattttcttggatttcttgataacaaggtagatgaggtttcacacaaatttgacgagcatgctccagcaccagccaagcaagtggttaacctggcccacgatttggtccaaaaggcaacccaaaaggctcagaaactagtagaggaggctcgaaccaatggagcacgaggggctttgcactacgctgcagctgagtacaagaacttggtgttggtaagctcgaccaagttgtgggttaaactgaacagcaacttgactttcaactcagtgacagaaaaggttgttcccaaagcagctaatttgtgtgagaaatacaacagttggttaaaggagaagagcggaaaaggttatcctgtggttgggtatttaccatcgattcccgttgatgcctttgggaaggctgttaaggaagctgaaggaaaagaggaagcaaatgcggatgatcataaatctgaatcagattcagattag

SEQ ID NO: 109 - protein, 231 aa, Gossypium hirsutum
MATMEKGLEFENKNLGLKHLNFVRVATIHALVCVSNLYDYSKQNSGPLRSTVDAVEHAVTTAVGPAYGKFKDVPDHFLGFLDNKVDEVSHKFDEHAPAPAKQVVNLAHDLVQKATQKAQKLVEEARTNGARGALHYAAAEYKNLVLVSSTKLWVKLNSNLTFNSVTEKVVPKAANLCEKYNSWLKEKSGKGYPVVGYLPSIPVDAFGKAVKEAEGKEEANADDHKSESDSD

(19) United States
(12) Patent Application Publication
(10) Pub. No.: US 2026/0176642 A1
(43) Pub. Date: Jun. 25, 2026

(54) COMPOSITIONS, METHODS, AND SYSTEMS FOR TARGETED MODIFICATION OF LIPID DROPLET-ASSOCIATED PROTEIN ACTIVITY IN PLANTS

(71) Applicant: UNIVERSITY OF NORTH TEXAS, Denton, TX (US)

(72) Inventors: Kent Chapman, Denton, TX (US); Payton Whitehead, Denton, TX (US)

(21) Appl. No.: 19/430,545

(22) Filed: Dec. 23, 2025

Related U.S. Application Data
(60) Provisional application No. 63/738,301, filed on Dec. 23, 2024.

(51) Int. Cl. C12N 15/82; C07K 14/415; C12N 9/02; C12N 9/10; C12N 9/88

(52) U.S. Cl. CPC C12N 15/8247; C07K 14/415; C12N 9/0008; C12N 9/1029; C12N 9/1051; C12N 9/88; C12N 15/8205; C12N 15/8207; C12Y 102/01084; C12Y 203/01075; C12Y 204/01143; C12Y 402/03061

(57) ABSTRACT

Described herein are compositions and methods relating to genetically modified plants and their production genetically modified plants. In certain aspects, genetically modified plants described herein have increased wax ester packaging from the endoplasmic reticulum into lipid droplets in the cytoplasm of the cells. In certain aspects, compositions and methods described herein can engineer genetic modifications in plants including a modified lipid droplet-associated protein (LDAP) gene and systems and methods for modifying the LDAP gene in a plant.

### CROSS REFERENCE TO RELATED APPLICATIONS

This application claims priority to, and the benefit of, U.S. provisional application entitled “METHODS AND SYSTEMS FOR TARGETED MODIFICATION OF LIPID DROPLET-ASSOCIATED PROTEIN ACTIVITY IN PLANTS” having Ser. No. 63/738,301, filed Dec. 23, 2024, which is hereby incorporated by reference in its entirety.

### STATEMENT REGARDING FEDERALLY SPONSORED RESEARCH

This disclosure was made with U.S. Government support under grant number DE-SC00016536, awarded by the Department of Energy (Office of Science, BES-Physical Biosciences Program). The U.S. government has certain rights in the disclosure.

### STATEMENT REGARDING COLOR DRAWINGS

In accordance with 37 C.F.R. § 1.84(a)(2)(ii), this application file contains at least one drawing executed in color. Copies of this patent or patent application publication with color drawing(s) will be provided by the United States Patent and Trademark Office upon request and payment of the necessary fee.

### SEQUENCE LISTING

The Sequence Listing submitted Dec. 23, 2025 as an ST.26.xml file named “921402_1230_SeqListing.xml,” created on Dec. 23, 2025 and having a size 177,620 bytes is hereby incorporated by reference as if fully set forth herein.

### BACKGROUND

Storing lipids is a universal and evolutionarily conserved feature across organisms found in all of biology. While storage mechanisms may differ between prokaryotes and eukaryotes, one common theme is that hydrophobic molecules are packaged into subcellular organelles known as lipid droplets (LDs). In plants, LDs are generally small in size (i.e., ˜1.0 μm) and composed of a neutral lipid core surrounded by a phospholipid monolayer derived from the endoplasmic reticulum (ER) membrane during a directional budding process. In many organisms, the primary neutral lipids that are stored in LDs are triacylglycerols (TAGs) and stearyl esters (SEs), but in some organisms the core of the LDs can vary, including wax esters (WEs), retinyl esters, or terpenes. Regardless of their composition, LDs were originally thought of as merely storage depots for lipophilic compounds, but have since been found to be linked to an array of functions including membrane trafficking, lipid signaling, and membrane remodeling during responses to stress and/or developmental changes.

While the overall mechanisms underlying LD biogenesis are still being uncovered, super-resolution microscopy and electron microscopy tomography have revealed that LD biogenesis begins at the endoplasmic reticulum (ER) membrane bilayer. Various proteins, including those involved in the final steps of neutral lipid synthesis, as well as various structural proteins specific to LD formation and maintenance, localize to distinct regions within the ER. While the role(s) of these LD structural proteins are still poorly understood, it is thought that some assist in the mitigation of membrane packaging defects, promotion of membrane curvature, and/or LD stabilization and prevention of LD-LD fusion. In addition, other proteins presumably promote the emergence and vectorial “budding” of the LD away from the ER surface, yielding a fully-formed, nascent LD that may be released into the cytoplasm via a scission event or may remain physically connected to the ER.

While the majority of plant LD biogenesis studies have focused primarily on the storage of TAGs, a number of plant species accumulate other types of lipid compounds, such as WEs, terpenes, terpene esters or TAGs containing unusual fatty acids. Many of these compounds are of economic interest due to their potential usage in high-value nutraceutical or industrial applications. However, they are often produced in plants with low yields or other agronomic limitations. Investigations into the underlying mechanisms responsible for the synthesis and accumulation of these lipids have revealed that differential expression and/or evolutionarily diverged forms of lipid biosynthetic enzymes are often involved in their production. Numerous attempts have been made to express these genes in higher yielding platform crops, but unfortunately, amounts of the desired lipids are often well below that needed for commercial applications and sometimes have negative effects on overall yield or development. Despite advances in LD biogenesis research, production of lipid compounds, such as WEs, in plants with high-yields for commercial applications is still desired.

### SUMMARY

In accordance with the purpose(s) of the disclosure, as embodied and broadly described herein, the disclosure, in one aspect, relates to methods of producing genetically modified plants with altered lipid droplet-associated protein (LDAP) activity. Also provided are genetically modified plants having engineered genetic modifications including altered LDAP activity and expression. The present disclosure also includes recombinant DNA constructs for genetic modification of a LDAP gene and other genes in a plant.

Various embodiments of the present disclosure identify the presence of certain specific sequences in the C-terminal region of LDAP proteins. Furthermore, various embodiments of the present disclosure have surprisingly shown that it is possible to increase the capacity of LDAP proteins to package wax esters from the endoplasmic reticulum into lipid droplets by targeted manipulation of these sequences to produce modified LDAP proteins. The modified LDAP proteins of the present disclosure can be expressed in cells, organisms, and in particular plants, to increase wax ester accumulation. The targeted manipulation of these sequences can also be advantageously and conveniently achieved by introducing relatively small changes to endogenous LDAP genes using gene-editing technologies, to increase wax ester packaging from the endoplasmic reticulum into the lipid droplets in the cells, organisms, and in particular plants.

Embodiments of methods of the present disclosure for producing a genetically modified plant include at least the steps of genetically manipulating a LDAP gene in the plant, wherein genetic manipulation of the LDAP gene results in increased packaging of wax esters from the endoplasmic reticulum into lipid droplets.

According to other aspects of the present disclosure, genetically modified plants are provided including plants made by the methods of the present disclosure. In embodiments, genetically modified plants of the present disclosure include a genetic modification comprising introducing a recombinant DNA construct to a plant or portion of a plant thereof or introducing at least one mutation in the nucleotide sequence of the C-terminal domain of the LDAP gene of the plant. The genetically modified plant is distinguishable from a corresponding wild type plant.

The present disclosure also provides systems for modifying LDAP genes in a plant. In embodiments, the system includes one or more recombinant dis-armed Agrobacterium plasmids where the plasmids include one or more engineered DNA sequences configured to modify a LDAP gene in a plant.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects are usable in all aspects of the disclosure taught herein. Furthermore, the individual features of the dependent claims, as well as all optional and preferred features and modifications of the described aspects are combinable and interchangeable with one another.

The present disclosure further provides recombinant DNA constructs for altering lipid droplet-associated protein (LDAP) activity in a plant. In various embodiments, a recombinant DNA construct can include an LDAP nucleotide sequence encoding an LDAP, or a truncated functional variant thereof, a promoter operably linked to the LDAP nucleotide sequence, and a terminator sequence operably linked at the LDAP nucleotide sequence downstream of the LDAP nucleotide sequence. In embodiments, the LDAP protein can be a chimera of an Arabidopsis LDAP1 (AtLDAP1) and a jojoba LDAP1 (ScLDAP1). In some embodiments, a C-terminal domain of the LDAP can have at least 80%, 85%, 90%, 95%, 96%, 97%, 98%, 99%, or any other value between approximately 80% and 100% sequence identity with a C-terminal domain of a ScLDAP1. The recombinant DNA construct can further include a nucleotide sequence encoding an ScLDAP1, an N. benthamiana LDAP2 (NbLDAP2), an LDAP1, an LDAP2, an LDAP3, or a functional variant of any thereof. In some embodiments, the promoter can be selected from Act1, HSP18.2, ScBV, RUBQ1, RUBQ2, CaMV35S, nos, P OsCon1, Ubi-1, MMV, SVBV, ocs, TBSV p19, TYLCV V2, and RbcS. In various embodiments, the LDAP nucleotide sequence encoding the LDAP can have SEQ ID NO:1.

In embodiments, the LDAP nucleotide sequence can have SEQ ID NO:104 or SEQ ID NO:108. In some embodiments, the LDAP nucleotide sequence can include at least 80% sequence identity with SEQ ID NO:1 or SEQ ID NO:33. In some embodiments, the LDAP nucleotide sequence can include at least 85%, 90%, 95%, 96%, 97%, 98%, 99%, or any other value between approximately 80% and 100% sequence identity with SEQ ID NO:1 or SEQ ID NO:33. In some embodiments, the LDAP nucleotide sequence can encode a protein that has SEQ ID NO:105 or 109. In some embodiments, the LDAP nucleotide sequence can encode a protein with at least 80% sequence identity with SEQ ID NO:26 or SEQ ID NO:34. In some embodiments, the LDAP nucleotide sequence can include at least 85%, 90%, 95%, 96%, 97%, 98%, 99%, or any other value between approximately 80% and 100% sequence identity with SEQ ID NO:26 or SEQ ID NO:34. The recombinant DNA construct can further include a nucleotide sequence encoding a Marinobacter aquaeolei fatty acyl reductase (MaFAR), a jojoba wax synthase (WS), a Fat-Induced Transcript 2 (FIT2), a Mannosyl (alpha-1,6-) glycoprotein beta-1,2-N-acetylglucosaminyltransferase (MmDGAT2), a Nicotiana benthamiana 5-Epi-Aristolochene Synthase (NbEAS), a Histidine-Aspartic Acid-Glutamic Acid-Leucine (HDEL) peptide sequence, a jojoba Fatty Acyl-CoA Reductase (ScFAR), a Seipin1, a Seipin2, an LDAP-interacting protein, an oleosin1, an oleosin5, an oleosin5-like protein, a wax synthase (WS), a Fatty Acyl-CoA Reductase, a Fatty Acid Elongase, or a functional variant of any thereof. The recombinant DNA construct can also include a nucleotide sequence encoding a fluorescent protein.

The present disclosure also provides a method for genetically modifying a plant, including introducing a DNA construct to a plant or portion of a plant thereof, where the plant is a Solanaceae plant or a Brassicaceae plant; or introducing at least one mutation in the nucleotide sequence of the C-terminal domain of the LDAP gene of the plant. In some embodiments, the Solanaceae plant can be a Gossypium hirsutum or a Nicotiana benthamiana. In some embodiments, the Brassicaceae plant can be an Arabidopsis, Camelina, pennycress, Physaira, B. carinata, or a Brassica napus. In various embodiments, the introducing can be any one of floral dipping with A. tumefaciens strain GV301, Agrobacterium-mediated transformation, particle bombardment, or microinjection. In some embodiments, the A. tumefaciens strain GV301 can include a WS gene and an ScLDAP gene. In some embodiments, the WS gene and the ScLDAP gene can be operably linked. In some embodiments, the A. tumefaciens strain GV301 can include an NbLDAP2 gene, an MmDGAT2 gene, a FIT2 gene, an NbEAS gene, or a combination of any thereof. In some embodiments, the NbLDAP2 gene, the MmDGAT2 gene, the FIT2 gene, the NbEAS gene, or the combination of any thereof can be operably linked. In some embodiments, the at least one mutation can result in a leucine at a position corresponding to position 161 in an AtLDAP in the C-terminal domain of the LDAP protein of the plant.

In various embodiments, a plant cell can include a recombinant DNA construct including a nucleotide sequence that encodes a lipid droplet-associated protein 1 (LDAP1) or a lipid droplet-associated protein 2 (LDAP2). In some embodiments, the recombinant DNA can be stably integrated. In embodiments, a plant cell can include a nucleotide sequence encoding a jojoba wax synthase (WS), a Marinobacter aquaeolei FAR (MaFAR), or a combination of any thereof. In some embodiments, a plant cell can include a nucleotide sequence encoding an MmDGAT2, an NbEAS, a FIT2, or a combination of any thereof. In various embodiments, a transgenic plant can include a plurality of the plant cells.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

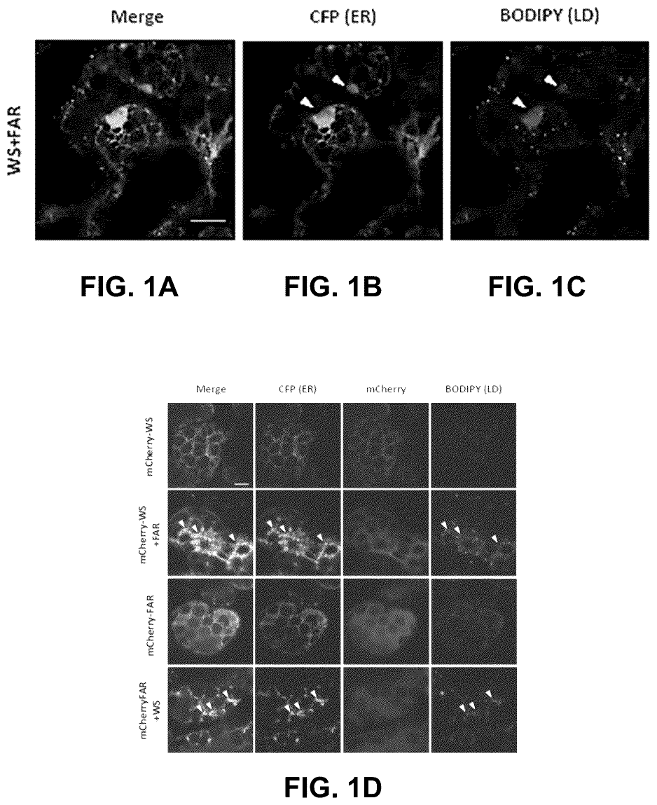

FIGS. 1A-1D show accumulation of wax esters (WEs) in the leaf tissue of N. benthamiana (a wild relative of common tobacco indigenous to Australia) induces ER defects. FIGS. 1A-1C show representative confocal laser scanning microscopic (CLSM) images (z-sections) of infiltrated N. benthamiana leaves transiently expressing jojoba wax synthase (WS), jojoba fatty acyl-CoA reductase (ScFAR), and a CFP tagged ER marker CFP-HDEL. Samples were stained with BODIPY (493/503) to visualize neutral lipids. Arrows point to regions of ER defects. Transient expression of WS and ScFAR induced large swollen regions of ER defects presumed to be caused by improper release of WEs from the endoplasmic reticulum (ER). Scale bars represent 10 μm. CFP-HDEL; cyan fluorescence, BODIPY (493/503); yellow fluorescence. FIG. 1D shows representative CLSM images (z-sections) of infiltrated N. benthamiana leaves transiently expressing mCherry tagged WS with CFP-HDEL, or both CFP-HDEL and untagged SCFAR; and mCherry tagged SCFAR transiently expressed in N. benthamiana with either CFP-HDEL, or both CFP-HDEL and untagged WS. Samples were stained with BODIPY (493/503) to visualize neutral lipids. Arrows point to regions of ER defects. Transient expression of either mCherry tagged WS or ScFAR showed expected cellular localization, and normal ER structuring. Co-expression of either fluorescently tagged WS or ScFAR with untagged ScFAR or WS respectively showed no alteration to the cellular localization of the proteins, but did induce the same BODIPY stained regions, further supporting the concept that the ER defects are induced by mispackaging of WEs. Arrows point to regions of ER defects presumed to be caused by the mispackaging of WEs. CFP-HDEL; cyan fluorescence, BODIPY (493/503); yellow fluorescence, mCherry; red fluorescence. Scale bar represents 10 μm. Transient expression of heterologous genes was accomplished by Agrobacterium-mediated infiltration. For each transient expression experiment Agrobacterium tumefaciens harboring the P19 viral suppressor construct was added to suppress transgene silencing. All samples were stained and imaged (Zeiss LSM710 confocal laser scanning microscope, fitted with AIRYSCAN attachment) at 5 days post infiltration.

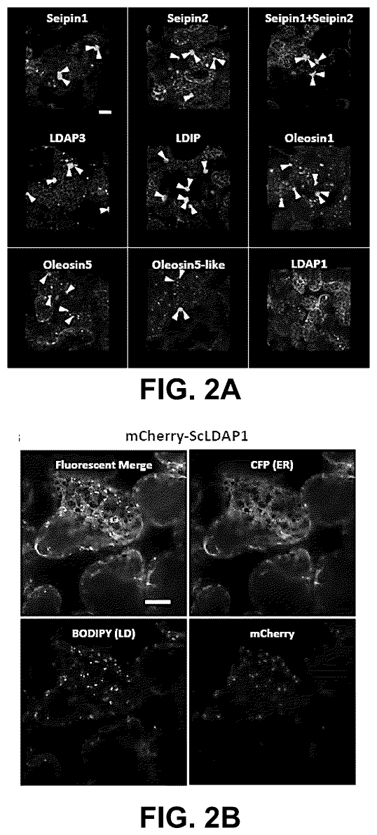

FIGS. 2A-2B show transient expression of jojoba lipid droplet packaging proteins and their capacity to correct WE induced ER defects. FIG. 2A shows representative CLSM images (z-sections) of infiltrated N. benthamiana leaves transiently expressing WS, SCFAR, and CFP-HDEL, in conjunction with jojoba lipid droplet (LD) proteins either individually, or in the case of Seipin1 and Seipin2, in combination with one another. All samples were stained with BODIPY (493/503) to visualize neutral lipids. Arrows point to regions of ER defects. Scale bar represents 20 μm. chlorophyll, blue fluorescence; BODIPY (493/503); yellow fluorescence. Of the jojoba LD proteins expressed with WS and ScFAR, only jojoba lipid droplet-associated protein 1 (ScLDAP1) showed the capacity to restore ER defects suggesting it has some involvement in WE packaging. FIG. 2B shows representative high resolution AIRYSCAN images of infiltrated N. benthamiana leaves transiently expressing WS, ScFAR, CFP-HDEL, and mCherry tagged jojoba LDAP1 (ScLDAP1). Samples were stained with BODIPY (493/503) to visualize neutral lipids. Expression of ScLDAP1 with WS and ScFAR showed “normal” reticulate ER structure and BODIPY stained LDs colocalizing with mCherry tagged ScLDAP1. CFP-HDEL; cyan fluorescence, BODIPY (493/503); yellow fluorescence, mCherry; red fluorescence. Scale bar represents 10 μm. Transient Expression of heterologous genes was accomplished by Agrobacterium-mediated expression. For each transient expression experiment A. tumefaciens harboring the P19 viral suppressor construct was added to suppress transgene silencing. All samples were stained and imaged (Zeiss LSM710 confocal laser scanning microscope, fitted with AIRYSCAN attachment) at 5 days post infiltration.

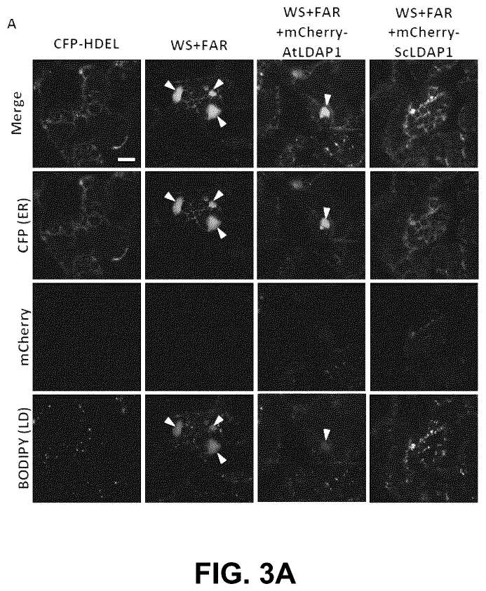

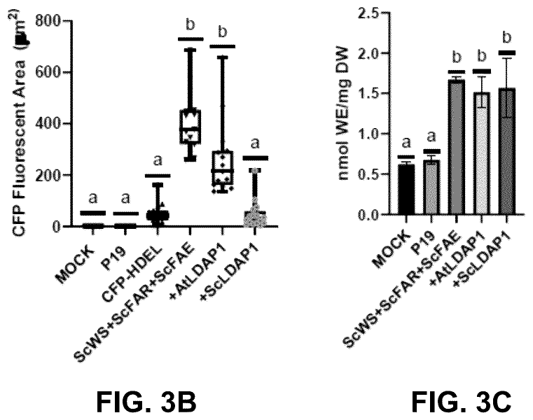

FIGS. 3A-3C show a comparison of jojoba and Arabidopsis LDAP1s' effect on ER structures and WE levels in N. benthamiana leaves. FIG. 3A shows representative CLSM images (z-sections) of infiltrated N. benthamiana leaves transiently expressing WS, ScFAR, CFP-HDEL, and either mCherry tagged Arabidopsis LDAP1 (AtLDAP1), or mCherry tagged jojoba (ScLDAP1). Samples were stained with BODIPY (493/503) to visualize neutral lipids. Arrows point to regions of ER defects. CFP-HDEL; cyan fluorescence, BODIPY (493/503); yellow fluorescence, mCherry; red fluorescence. Scale bar represents 10 μm. Transient expression of ScLDAP1 showed a restored reticulate ER structure, while AtLDAP1 showed no restoration suggesting a species-specific WE specificity of ScLDAP1. FIG. 3B shows the quantification of the CFP fluorescent area (μm²) associated with swollen ER structures. Values correspond to the averages of three individual experiments (with five images from each replicate). Different letters indicate significant difference at p≤0.05, as determined by one-way ANOVA with Tukey's post-test. FIG. 3C shows the quantification of the total amount of WEs produced in N. benthamiana leaves analyzed by electrospray ionization-mass spectrometry (ESI-MS). Asterisks indicate significant difference in comparison with the Mock control as determined by one-way ANOVA with Tukey's post-test (n=3, **p≤0.01). Transient expression of heterologous genes was accomplished by Agrobacterium-mediated transient expression. For each transient expression experiment A. tumefaciens harboring the P19 viral suppressor construct was added to suppress transgene silencing. For each transient expression experiment A. tumefaciens harboring a jojoba fatty acid elongase (FAE) was included to elongate endogenous Tobacco fatty acids to 20 and 22 carbon in length to mimic endogenous jojoba fatty acids.

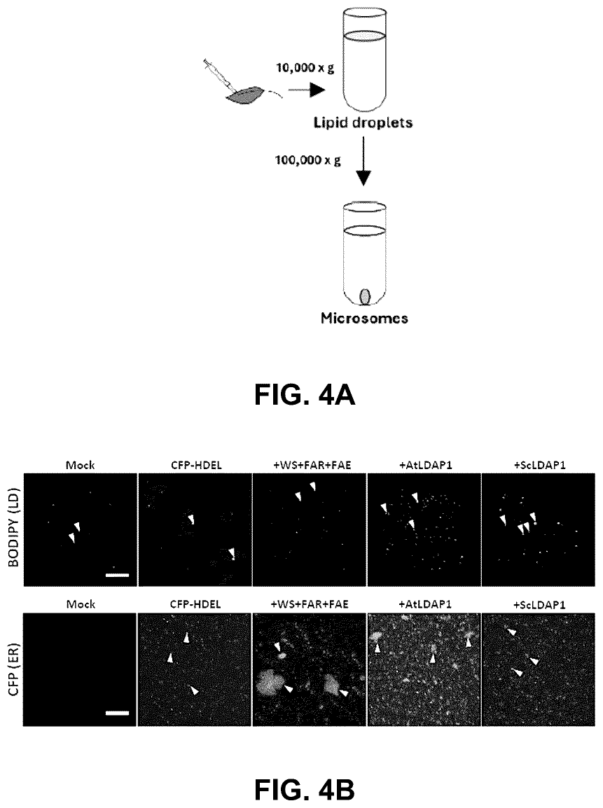

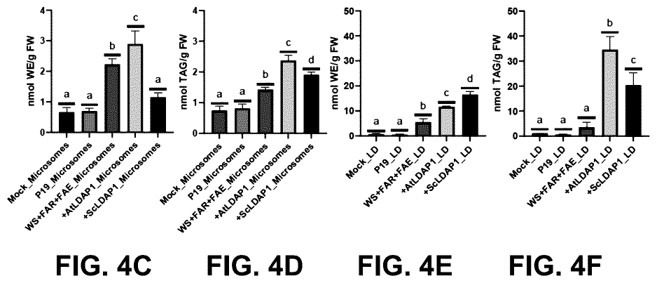

FIGS. 4A-4F show the analysis of WE distribution between Isolated LDs and microsomes from infiltrated N. benthamiana leaves. FIG. 4A is an illustration showing LDs from N. benthamiana leaf tissues were isolated by floatation centrifugation, and microsomes were pelleted from the remaining supernatant by ultracentrifugation. FIG. 4B shows representative CLSM images (single plane) of LDs and microsomes isolated from infiltrated N. benthamiana leaves. All isolated LD fractions were stained with BODIPY (493/503) to visualize neutral lipids. Arrows point to BODIPY stained LDs or CFP tagged microsomes. Scale bar represents 40 μm. CFP-HDEL; cyan fluorescence, BODIPY (493/503); yellow fluorescence. Addition of either AtLDAP1 or ScLDAP1 showed an increase in the number of LDs, but only ScLDAP1 showed a decrease in the size of CFP tagged structures. FIGS. 4C-4F show the quantification and analysis of percent distribution of either WEs or TAGs between LD and microsomal fractions isolated from infiltrated N. benthamiana leaves. Different letters indicate significant difference at p≤0.05, as determined by one-way ANOVA with Tukey's post-test (n=3). Transient expression of heterologous genes was accomplished by Agrobacterium-mediated expression. For each transient expression experiment A. tumefaciens harboring the P19 viral suppressor construct was added to suppress transgene silencing. For each transient expression experiment A. tumefaciens harboring a jojoba fatty acid elongase (FAE) was included to elongate endogenous Tobacco fatty acids to 20 and 22 carbon in length to mimic endogenous jojoba fatty acids.

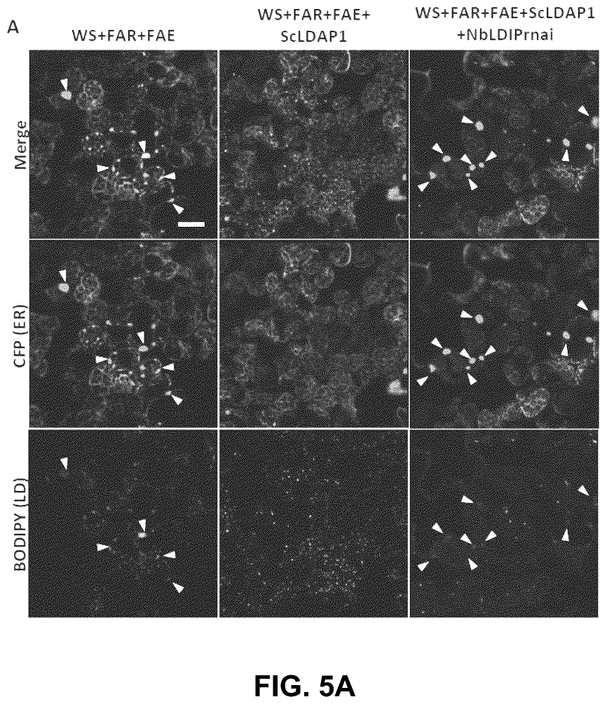

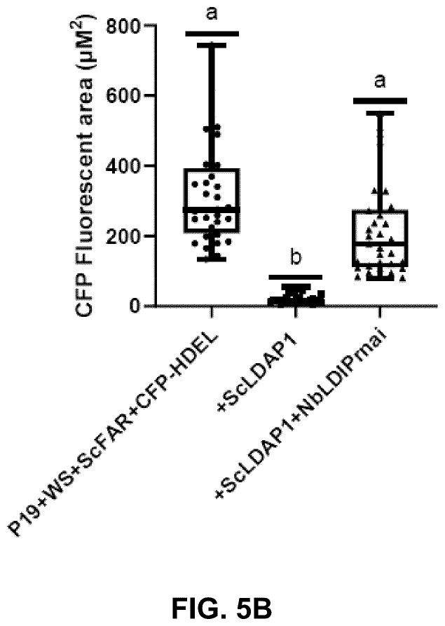

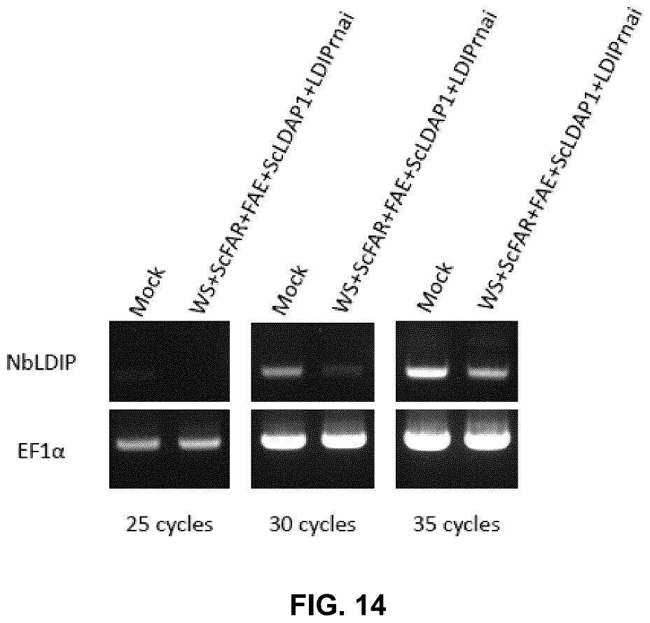

FIGS. 5A-5B show RNA-interference based suppression of N. benthamiana LDIP (LDAP-interacting protein) shows a loss of ScLDAP1 WE specific function, indicating that LDAP function is dependent on adequate availability of LDIP. FIG. 5A shows representative CLSM images of N. benthamiana transiently expressing WS, ScFAR, CFP-HDEL, and either ScLDAP1, or ScLDAP1 and N. benthamiana LDIP RNAi suppressor (NbLDIPrnai). Samples were stained with BODIPY (493/503) to visualize neutral lipids. Arrows point to regions of ER defects. Scale bar corresponds to 40 μm. CFP-HDEL; cyan fluorescence, BODIPY (493/503); yellow fluorescence, chlorophyll; blue fluorescence. Transient expression of heterologous genes was accomplished by Agrobacterium-mediated expression. For each transient expression experiment A. tumefaciens harboring the P19 viral suppressor construct was added to suppress transgene silencing. All samples were stained and imaged (Zeiss LSM710 confocal laser scanning microscope, fitted with AIRYSCAN attachment) at 5 days post infiltration. CFP quantification was based on a minimum of 29 images from three replicate infiltration experiments for each treatment. Confirmation of RNAi suppression of NbLDIP was evaluated by RT-PCR (FIG. 14) RNAi suppression of NbLDIP caused a reversion of ScLDAP1 restored ER back to swollen ER defects. This suggests that LDIP can be utilized for ScLDAP1 to perform its WE specific function. FIG. 5B shows the quantification of the CFP fluorescent area (μm²) associated with swollen ER defects. Values correspond to the averages of three individual infiltration experiments (with a minimum of 10 images taken from 3 separate infiltrations). Asterisks indicate significant difference in comparison between each sample as determined by one-way ANOVA with Kruskal-Wallis (****p≤0.0001).

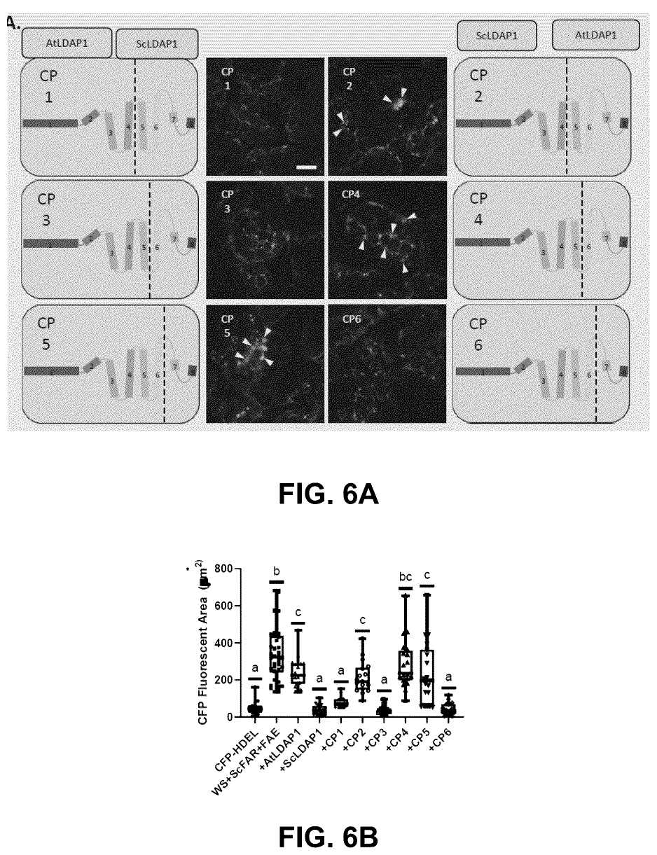

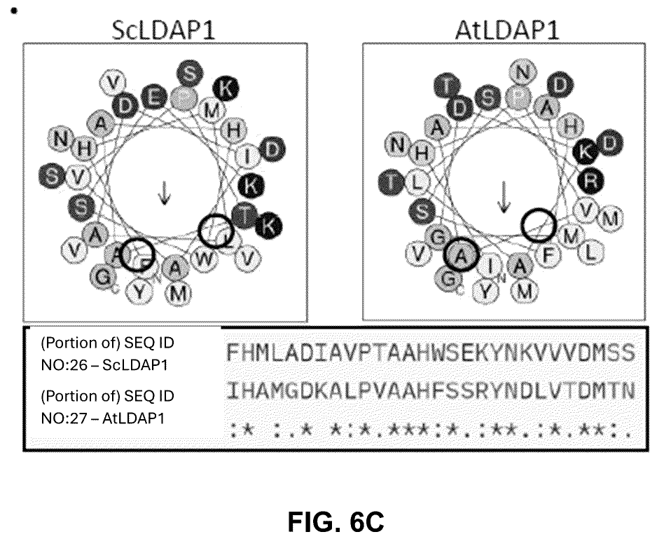

FIGS. 6A-6C show that chimeric LDAP1s expression shows WE-induced ER defect restoration. FIG. 6A shows representative high resolution AIRYSCAN images of infiltrated N. benthamiana leaves transiently expressing WS, ScFAR, CFP-HDEL, and mCherry tagged chimeric LDAP1s. Samples were stained with BODIPY (493/503) to visualize neutral lipids. Arrows point to regions of ER defects. Scale bar represents 10 μm. CFP-HDEL; cyan fluorescence, BODIPY (493/503); yellow fluorescence, mCherry; red fluorescence. For each transient expression experiment A. tumefaciens harboring the P19 viral suppressor construct was added to suppress transgene silencing. All samples were stained and imaged (Zeiss LSM710 confocal laser scanning microscope, fitted with AIRYSCAN attachment) at 5 days post infiltration. Cartoon diagrams illustrate regions of chimeric swaps between Arabidopsis and jojoba LDAP1 indicated by a dotted line. Cartoons representing secondary structures (rectangles, alpha helices; lines, disordered loops) are colored in a rainbow scheme starting from the N-terminus to C-terminus of the protein. Based on the expression of each chimeric protein, one helix from jojoba LDAP1 (colored yellow) has been identified that seems to affect its ability to correct WE-induced ER defects. FIG. 6B shows quantification of the CFP fluorescent area (μm²) associated with swollen ER defects. Values correspond to the averages of three individual experiments (with ˜7 images from each replicate). Different letters indicate significant difference at p≤0.05, as determined by one-way ANOVA with Tukey's post-test. FIG. 6C shows the helical wheel projection of the alpha helices from both AtLDAP1 and ScLDAP1 (generated by Heliquest webserver). Hydrophobic residues are colored yellow, hydrophilic and charged residues are colored purple, blue, pink, red and blue. Alanine and glycine residues are colored grey. Proline residues are colored green. The direction of the arrowhead in the center of the wheel indicates the position of the hydrophobic face along the axis of the helices. Black circles indicate residues that have been identified as being unique to both ScLDAP1 and AtLDAP1, suggesting some involvement in ScLDAP1s WE specific function, and AtLDAP1s lack of WE specificity.

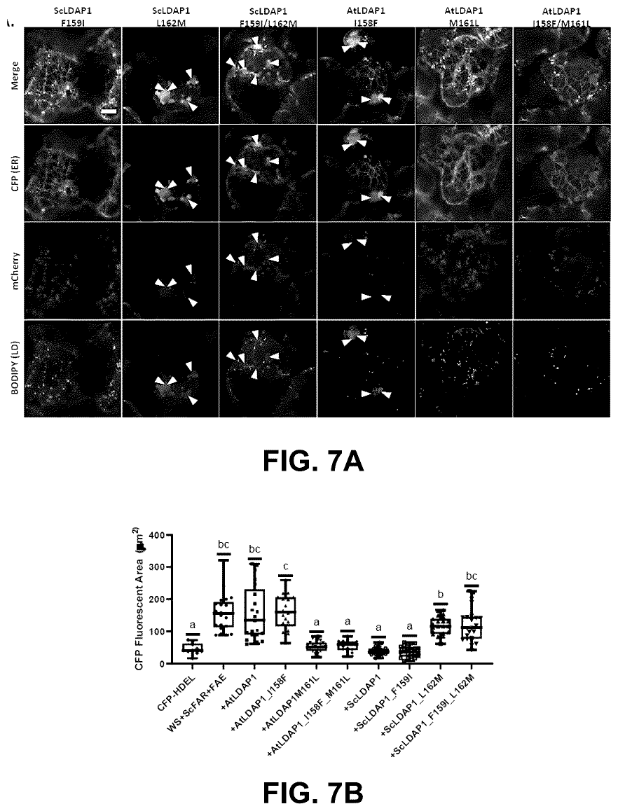

FIGS. 7A-7B show the mutant LDAP1 expression shows a gain and loss of WE specificity for both AtLDAP1 and ScLDAP1. FIG. 7A shows representative high resolution AIRYSCAN images of infiltrated Tobacco leaves transiently expressing WS, ScFAR, FAE, CFP-HDEL, and mCherry tagged mutant LDAP1s. Samples were stained with BODIPY (493/503) to visualize neutral lipids. Arrows point to regions of ER defects. All samples were stained and imaged (Zeiss LSM710 confocal laser scanning microscope, fitted with AIRYSCAN attachment) at 5 days post infiltration. Mutation of AtLDAP1 residue 161 from methionine (M) to Leucine (L) showed a gain of WE specificity, while the comparable mutation of ScLDAP1 residue 161 from L to M showed a loss of WE specificity. Mutations of AtLDAP1 residue 158 from Isoleucine (1) to phenylalanine (F), and ScLDAP1 residue 159 from F to I did not show any effect on WE specificity. Double mutations of LDAP1s from both Arabidopsis and jojoba showed a similar gain and loss of function that was shown with the singular L and M mutations. FIG. 7B shows the quantification of the CFP fluorescent area (μm²) associated with swollen ER defects. Values correspond to the multiple images collected from each of three individual experiments (with ˜7 images from each replicate). Different letters indicate significant difference at p≤0.05, as determined by one-way ANOVA with Tukey's post-test.

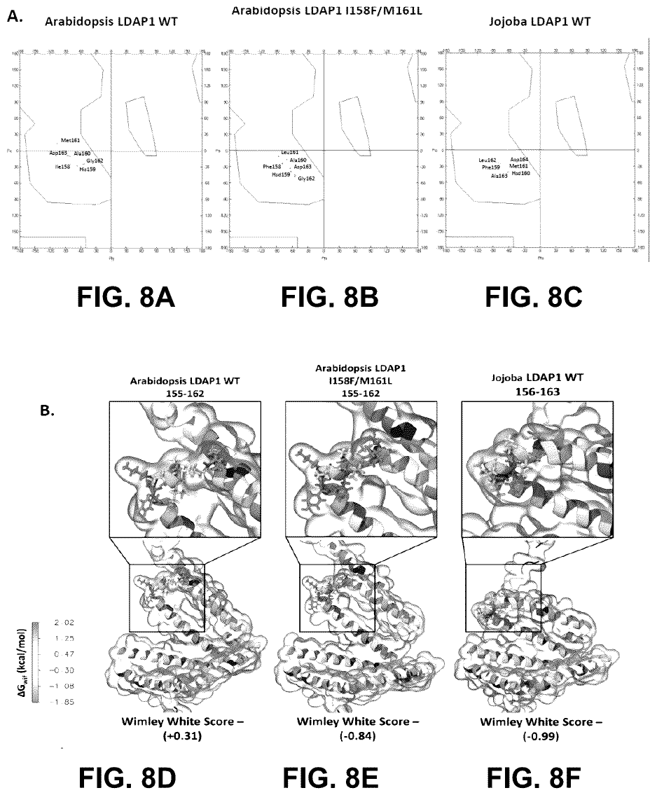

FIGS. 8A-8F show the structural and hydrophobic differences of wild type (WT) and mutant LDAP1. FIGS. 8A-8C show the Ramachandran plots analyzing the phi and psi angles of Arabidopsis LDAP1 WT (FIG. 8A), Arabidopsis LDAP1 I158F/M161L (FIG. 8B), and jojoba LDAP WT (FIG. 8C). Dots shown are the residues in the loops where the protein interacts with the membrane. The blue and yellow outlined regions represent the marginally allowed and allowed regions for dihedral angle distribution respectively. FIGS. 8D-8F show the predicted and highlighted secondary structures for AtLDAP (FIG. 8D), AtLDAP1 I158F/M161L (FIG. 8E), and ScLDAP1 (FIG. 8F). Hydrophobicity measurements using the Wimley-White scale of amino acids are shown for these amplified regions. Blue or positive values correspond to higher free energy values (hydrophilicity), while red or negative values indicate lower free energy values (hydrophobicity). Wimley-White score comparison of selected residues in the region of LDAP1s shown to be important for WE specific function show jojoba LDAP1 and Arabidopsis I158F/M161L mutant LDAP1 as having a more favorable free energy value compared to WT Arabidopsis LDAP1, suggesting more favorable associations with the wax-containing LD monolayer.

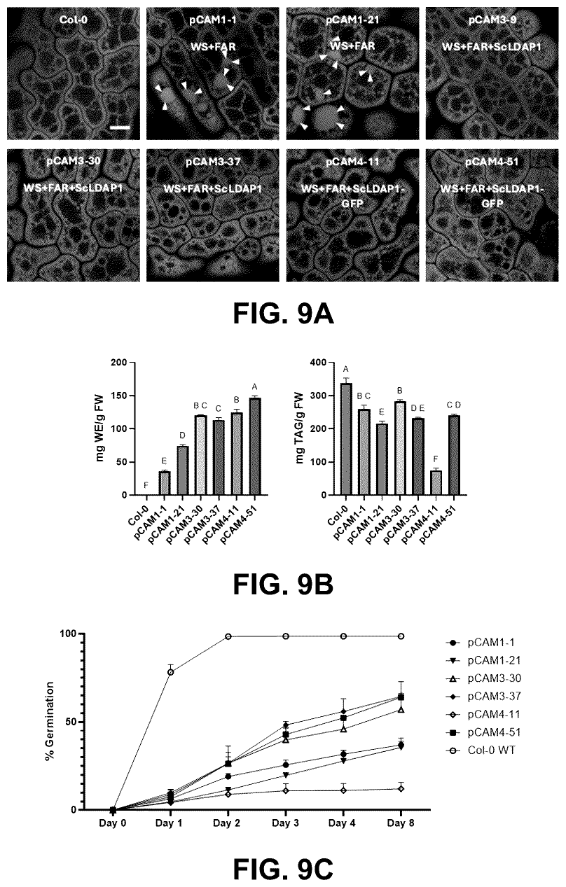

FIGS. 9A-9C show that ScLDAP1 improves WE partitioning and accumulation in stable transgenic Arabidopsis lines. FIG. 9A shows representative high resolution AIRYSCAN images of Arabidopsis seed embryos stably expressing ScWS, MaFAR, and ScLDAP1. Samples were stained with BODIPY (493/503) to visualize neutral lipids. Arrows point to regions of lipid aggregates. BODIPY (493/503); green fluorescence. Scale bar represents 5 μm. AIRYSCAN imaging of Arabidopsis seed embryos expressing ScWS and MaFAR show large amorphous lipid-stained structures similar to those seen in N. benthamiana leaves expressing the same genes. The expression of ScLDAP1 in conjunction with ScWS and MaFAR showed a more uniform LD phenotype with no large lipid-stained structures. FIG. 9B shows WE and TAG quantification of transgenic Arabidopsis lines expressing ScWS and MaFAR with or without ScLDAP1. Quantification represents mean values of triplicate analysis of each line (n=3). Different letters indicate significant difference at p≤0.05, as determined by one-way ANOVA with Tukey's post-test Samples labeled pCAM1 correspond to WE and TAG amounts from lines expressing just ScWS and MaFAR. Samples labeled pCAM3 correspond to WE and TAG amounts from lines expressing ScWS, MaFAR, and untagged ScLDAP1. Samples labeled pCAM4 correspond to WE and TAG amounts from lines expressing ScWS, MaFAR, and C-terminal GFP tagged ScLDAP1. Several lines expressing ScLDAP1 showed a noticeable increase in the amount of WE that is accumulated compared to lines that only express ScWS and MaFAR. FIG. 9C shows the percent seed germination of Arabidopsis seeds expressing either ScWS and MaFAR individually, or in conjunction with ScLDAP1. Seeds were considered to be germinating by radical emergence. Three replicate germinations of a minimum of 150 seeds were used per line. Number of germinating seeds were counted at 1, 2, 3, 4, and 8 days post exposure to light.

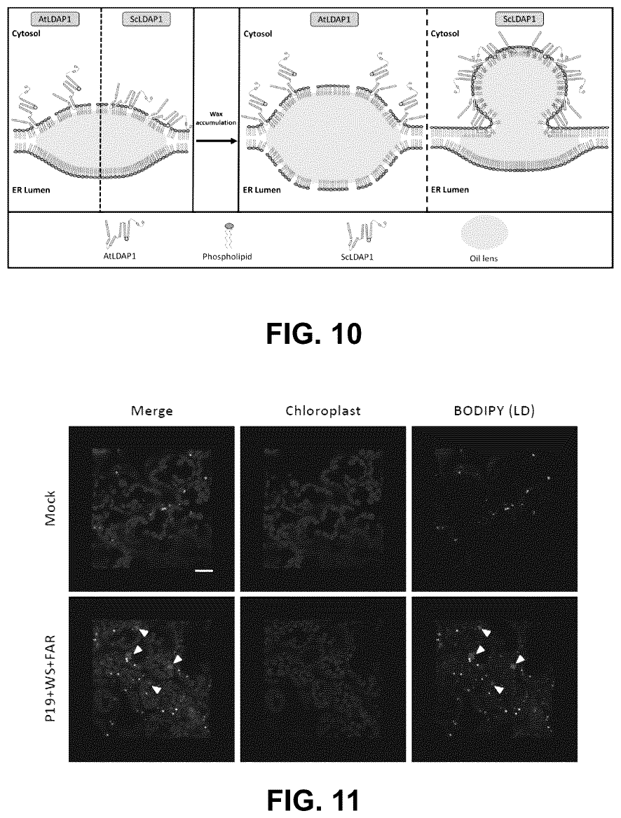

FIG. 10 shows a model for ScLDAP1 WE specific function. The model depicts the proposed effects of ScLDAP1 and AtLDAP1 on WE LD formation. Both AtLDAP1 and ScLDAP1 associate to sites of LD biogenesis through a helix-loop region near the N-terminus of the protein that recognizes the monolayer phospholipid surface with underlying WE (left). Further WE accumulation proceeds, but it remains trapped in the ER because the AtLDAP1 lacks a wax-ester specific sensor region near helix 6 (middle). On the other hand, the jojoba isoform ScLDAP1 has a hydrophobic sensor region near helix 6 that provides for a secondary interaction with the monolayer wax ester surface, providing for correct packaging of LDs and facilitating their export from the ER (right).

FIG. 11 shows WE induced BODIPY stained defects form independent of CFP-HDEL expression. Representative CLSM images of N. benthamiana leaves transiently expressing WS, and ScFAR. Samples were stained with BODIPY (493/503) to visualize neutral lipids. Arrows point to large BODIPY stained structures. For each transient expression experiment A. tumefaciens harboring the P19 viral suppressor construct was added to suppress transgene silencing. All samples were stained and imaged (Zeiss LSM710 confocal laser scanning microscope, fitted with AIRYSCAN attachment) at 5 days post infiltration. Chlorophyll autofluorescence, blue fluorescence; BODIPY (493/503); yellow fluorescence. Scale bars represent 20 μm. The appearance of large BODIPY stained structures upon expression of WS and ScFAR support the hypothesis that ER defects and neutral lipid accumulation in the ER is a result of WE miss-packaging and not protein aggregation.

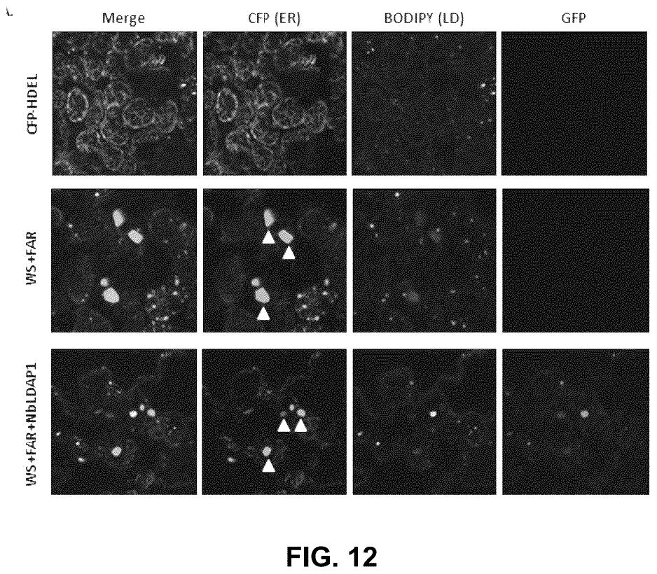

FIG. 12 shows transient expression of N. benthamiana LDAP1 does not correct WE-induced defects. Representative CLSM images of N. benthamiana leaves transiently expressing WS, ScFAR, CFP-HDEL (ER), and N-terminal GFP tagged N. benthamiana LDAP1 (NbLDAP1). Samples were stained with BODIPY (493/503) to visualize neutral lipids. Arrows point to regions of ER defects. For each transient expression experiment A. tumefaciens harboring the P19 viral suppressor construct was added to suppress transgene silencing. All samples were stained and imaged (Zeiss LSM710 confocal laser scanning microscope, fitted with AIRYSCAN attachment) at 5 days post infiltration. Scale bars represent 20 μm. Transient expression of NbLDAP1 in conjunction with WE accumulation showed no restoration of WE-induced defects.

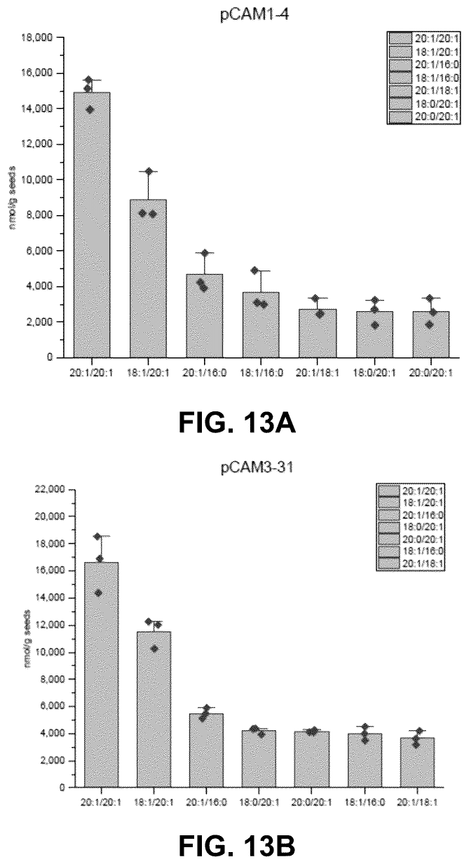

FIGS. 13A-13B show ScLDAP1 expression does not affect WE profiles in Arabidopsis seeds expressing WE machinery. Molecular species of WEs in seeds of representative Arabidopsis lines expressing MaFAR/WS (pCAM1-4) (FIG. 13A) and MaFAR/WS/ScLDAP1 (pCAM3-31) (FIG. 13B). WE composition was determined by nano-ESI-MS/MS. Shown is the mean content (+SD; n=3) of the top seven WE molecular species (alcohol moiety/acyl moiety).

FIG. 14 shows confirmation of NbLDIP silencing in N. benthamiana leaves by qualitative RT-PCR. Qualitative RT-PCR reactions from RNA extracted from either mock infiltrated, or listed construct infiltrated N. benthamiana leaves. Primers specific to endogenous NbLDIP were used for amplification at 25, 30, and 35 cycles. N. benthamiana elongation factor 1α (EF1α) was used as a reference gene with its primers listed in Sequences. Top row of gel images corresponds to amplification of NbLDIP. Bottom row of gel images corresponds to amplification of EF1α.

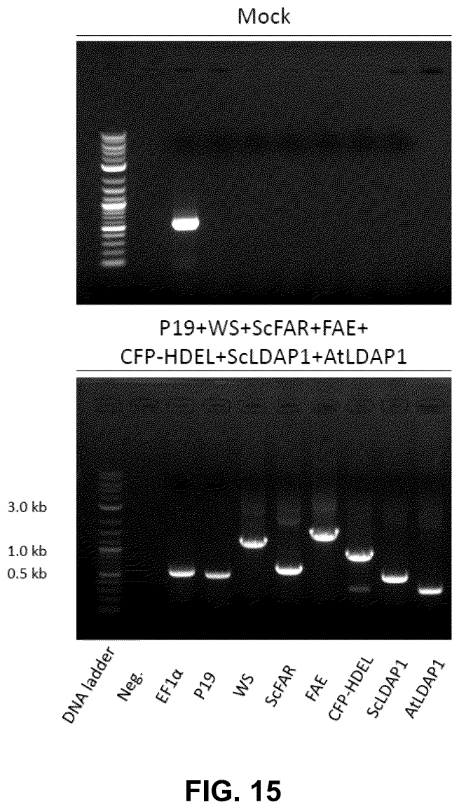

FIG. 15 shows confirmation of transgene expression by qualitative RT-PCR. Transient expression of transgenes (P19, WS, ScFAR, FAE, CFP-HDEL, ScLDAP1, and AtLDAP1) was confirmed by qualitative RT-PCR. EF1α was used as a reference gene for each sample. Top gel shows only the expression of EF1α and not transgenes in mock control. Bottom gel shows expression of all transgenes as well as EF1α. Negative control (Neg.) reactions with EF1α specific primers using RNA samples not treated with reverse transcriptase were shown to confirm no DNA contaminations in the samples.

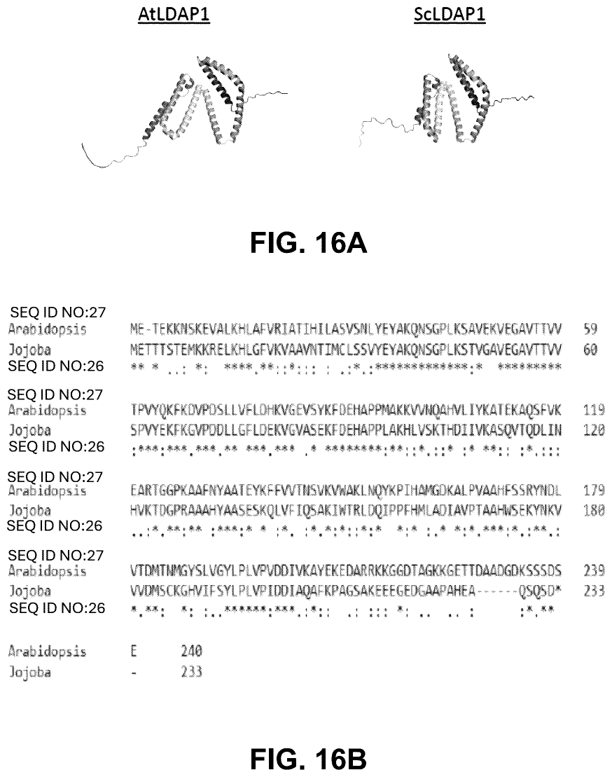

FIGS. 16A-16B show Alphafold2 modeling, and Sequence Alignment of AtLDAP1 and ScLDAP1. FIG. 16A shows Alphafold predicted structural models of AtLDAP1 and ScLDAP1. Blue to red coloring of models follows N-terminal to C-terminal residue progression. FIG. 16B shows sequence alignment of the deduced peptide sequences of LDAP1 from A. thaliana (Arabidopsis), and S. chinensis (jojoba). Numbers to the right of the sequences indicate residue position in the respective peptide. Asterisks underneath residue indicate conservation of residues between the two peptides at that position. Colons underneath residues indicate conservation of residues with similar properties at that position. Periods indicate conservation of residue with weakly similar properties at that position.

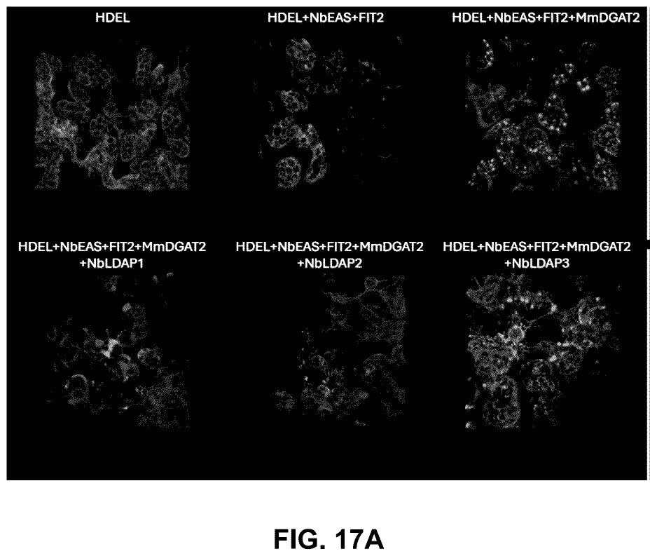

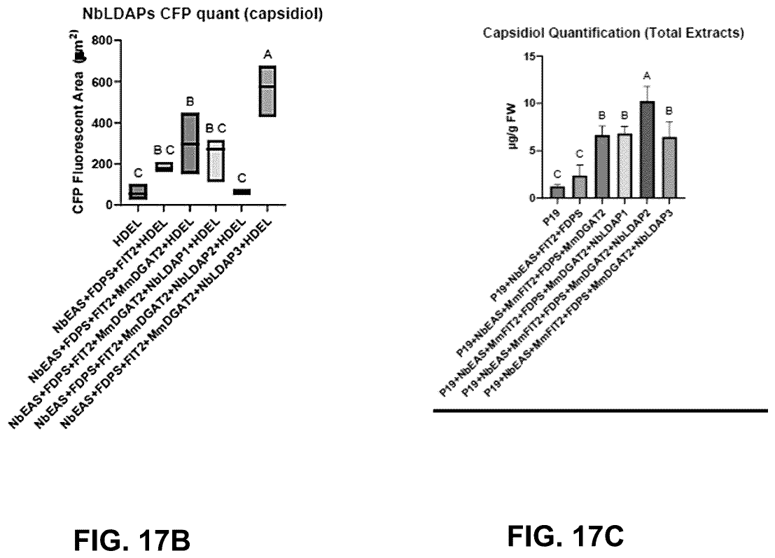

FIGS. 17A-17C show a comparison of the effects of N. benthamiana LDAP1 (NbLDAP1), NbLDAP2, and NbLDAP3 on ER structures and capsidiol levels in N. benthamiana leaves. FIG. 17A shows representative CLSM images (z-sections) of infiltrated N. benthamiana leaves transiently expressing HDEL, NbEAS, FIT2, MmDGAT2, and one of NbLDAP1, NbLDAP2, or NbLDAP3. CFP-HDEL; cyan fluorescence. Scale bar represents 10 μm. Transient expression of NbLDAP2 showed a restored reticulate ER structure, while NbLDAP1 and NbLDAP2 showed little to no restoration. FIG. 17B shows the quantification of the CFP fluorescent area (μm²) associated with swollen ER structures. Values correspond to the averages of three individual experiments. Different letters indicate significant difference at p≤0.05, as determined by one-way ANOVA with Tukey's post-test. FIG. 17C shows the quantification of the total amount of capsidiol produced in Tobacco leaves by ESI-MS analysis. Transient expression of heterologous genes was accomplished by Agrobacterium-mediated expression. For each transient expression experiment A. tumefaciens harboring the P19 viral suppressor construct was added to suppress transgene silencing.

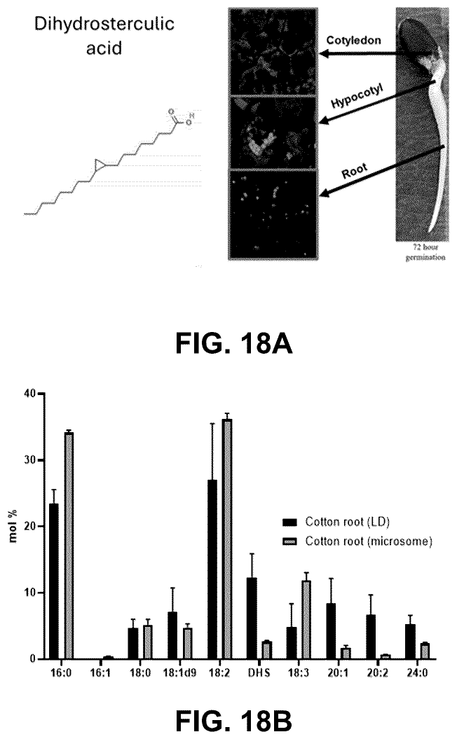

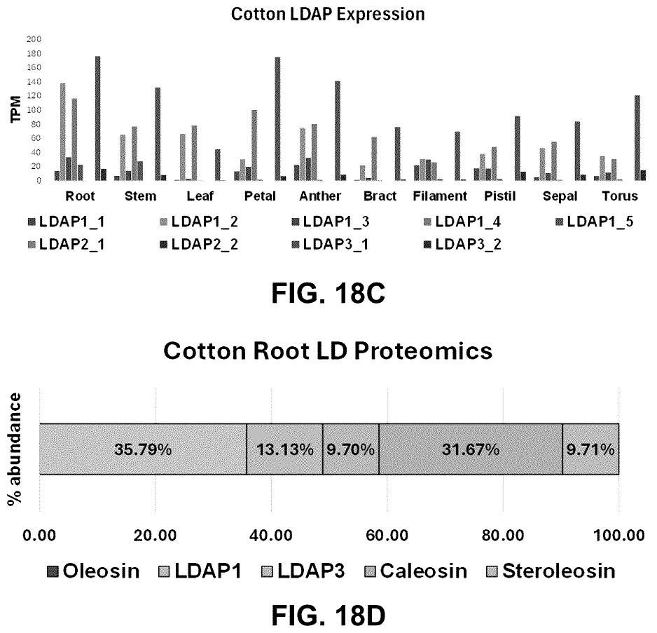

FIGS. 18A-18D show germinating cotton roots accumulate CFA rich lipid droplets that may require specialized LDAPs. FIG. 18A shows the chemical structure of a cyclopropane fatty acid (CFA) and representative CLSM images (z-sections) of BODIPY 493/503 stained hand sections of 3 day old germinating cotton seeds. Cotyledon, hypocotyl, and root sections were sectioned stained and images. Lipid droplet (LD) size and number differ significantly between tissues. FIG. 18B shows fatty acid methyl ester mol % quantification from germinating cotton root LD and microsome cellular fractions. CFAs are enriched in LD fractions isolated from germinating cotton roots. FIG. 18C shows the proteomic analysis of major LD proteins abundance associated with germinating cotton root LDs. Percent amount derived from averaged intensity of each isoform in the five LD protein families listed (n=3). Percent abundance of LDAP1 and LDAP3 in isolated cotton root LD proteome is similar to that of isolated lipid droplets from wax ester accumulating tissues from jojoba. FIG. 18D shows the expression profiles of LDAP1 and LDAP3 isoforms in various cotton tissues. Profiles derived from cottonMD multiomics analysis tool. Expression levels are plotted as TPM. Abbreviations: CFA, cyclopropane fatty acid; CLSM, confocal laser scanning microscopy; LD, lipid droplet. The expressions of LDAP3_1, LDAP1_1, and LDAP1_2 are shown to be higher in CFA accumulating tissues (root, stem), and could suggest that they are involved in the packaging of CFAs.

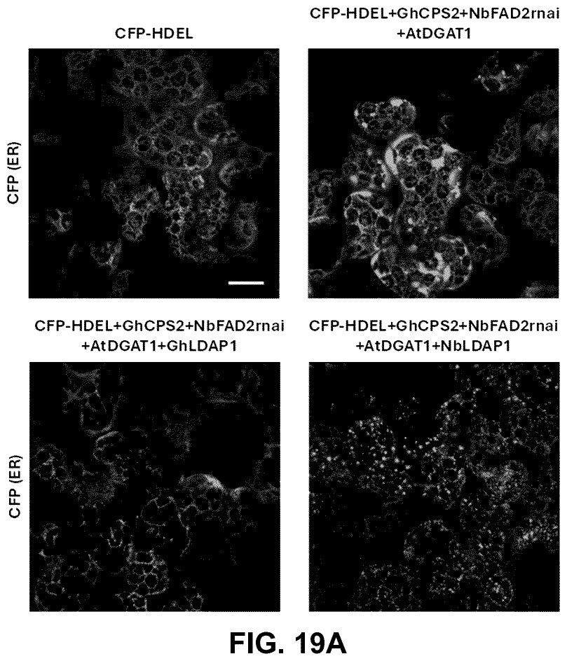

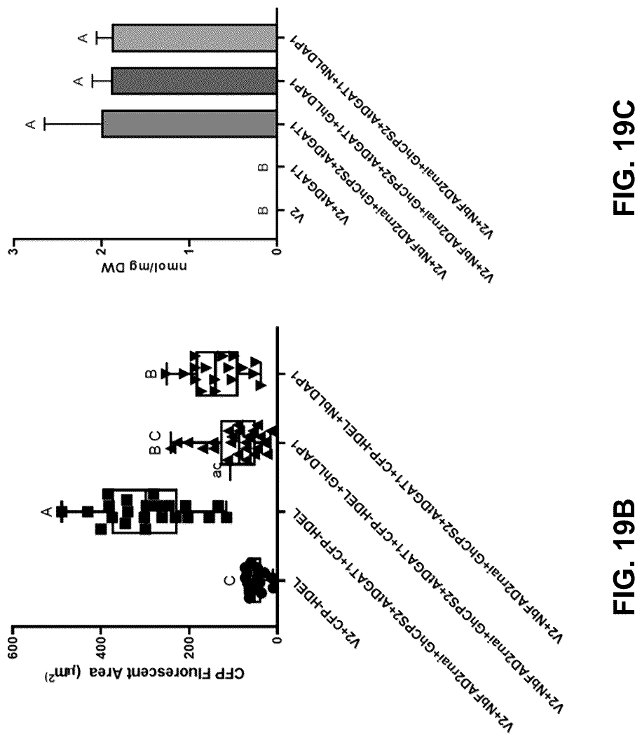

FIGS. 19A-19C show expression of G. hirsutum LDAP1 is more efficient at restoring CFA induced ER defects. FIG. 19A shows representative CLSM images (z-sections) of infiltrated N. benthamiana leaves transiently expressing the ER marker CFP-HDEL with and without GhCPS2, FAD2rnai, AtDGAT1, and either GhLDAP1 or NbLDAP1. Arrows point to regions of ER defects. GhLDAP1 expression showed the most significant restoration of ER defects. Scale bars represent 20 μm. CFP-HDEL; cyan fluorescence. Expression of either GhLDAP1 or NbLDAP1 showed a significant restoration of CFA induced ER defects. FIG. 19B shows quantification of the CFP fluorescent area (μm²) associated with swollen ER structures. Values correspond to the averages of three individual experiments (with four images from each replicate). Different letters indicate significant difference at p≤0.05, as determined by one-way ANOVA with Tukey's post-test. While neither GhLDAP1 nor NbLDAP1 seemed to be capable of restoring ER defects back to normal levels, both showed a partial restoration. FIG. 19C shows relative DHSA quantification from N. benthamiana leaf total extracts by GC-MS analysis. Different letters indicate significant difference at p≤0.05, as determined by one-way ANOVA with Tukey's post-test (n=3). Expression of either GhLDAP1 or NbLDAP1 did not increase the amount of CFA produced.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure.

Embodiments of the present disclosure will employ, unless otherwise indicated, techniques of molecular biology, genetics, biochemistry, botany, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20-25° C. and 1 atmosphere.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

Prior to describing the various aspects of the present disclosure, the following definitions are provided and should be used unless otherwise indicated. Additional terms may be defined elsewhere in the present disclosure.

### A. DEFINITIONS

In describing and claiming the disclosed subject matter, the following terminology will be used in accordance with the definitions set forth below. As used herein, the term “and/or” includes any and all combinations of one or more of the associated listed items. Expressions such as “at least one of,” when preceding a list of elements, modify the entire list of elements and do not modify the individual elements of the list.

It must be noted that, as used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

As used herein with reference to the relationship between DNA, cDNA, mRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “DNA molecule” can include nucleic acids/polynucleotides that are made of DNA.

As used herein, the term “encode” refers to principle that DNA can be transcribed into RNA, which can then be translated into amino acid sequences that can form proteins.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. As used herein, “identity,” can refer to a relationship between two or more nucleotide or polypeptide sequences, as determined by comparing the sequences. In the art, “identity” can also refer to the degree of sequence relatedness between nucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides of the present disclosure, unless stated otherwise. The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein. One of skill will recognize that these values can be appropriately adjusted to determine corresponding identity of proteins encoded by two nucleotide sequences by taking into account codon degeneracy, amino acid similarity, reading frame positioning and the like.

The term “LDAP” as used herein means lipid droplet-associated protein.

The term “unmodified LDAP” as used herein typically means a naturally occurring or native LDAP. In some cases, the LDAP sequence may have been assembled from sequences in the genome of a plant but may not be expressed in the plants.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “operatively linked” in the context of recombinant DNA molecules, vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms also contemplate plants, fungi, bacteria, etc.

As used herein, the terms “optional” or “optionally” means that the subsequently described event or circumstance may or may not occur, and that the description includes instances where said event or circumstance occurs and instances where it does not.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “promoter” includes all sequences capable of promoting or otherwise modulating transcription of a coding or a non-coding sequence. In particular, the term “promoter” as used herein refers to a nucleotide sequence generally, but not always, described as the 5′ regulator region of a gene, located proximal to the start codon, but can also include additional sequences influencing transcript of the gene. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two or more different proteins or protein fragments), the combination of a nucleic acid encoding a polypeptide to heterologous sequence (e.g., a regulatory sequence such as, but not limited to, a promoter sequence, where the coding sequence and heterologous sequence are from different sources or otherwise do not typically occur together naturally, such as a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “selectable marker” refers to a gene whose expression allows one to identify cells that have been transformed or transfected with a vector containing the marker gene. For instance, a recombinant nucleic acid may include a selectable marker operatively linked to a gene of interest and a promoter, such that expression of the selectable marker indicates the successful transformation of the cell with the gene of interest.

A “suitable control” is a control that will be appreciated by one of ordinary skill in the art as one that is included such that it can be determined if the variable being evaluated has an effect, such as a desired effect or hypothesized effect. One of ordinary skill in the art will also appreciate based on inter alia, the context, the variable(s), the desired or hypothesized effect, what is a suitable or an appropriate control needed.

As used herein, “transforming” (also “transformation” or “transformed”) when used in the context of engineering or modifying a cell, refers to the introduction by any suitable technique and/or the transient or stable incorporation and/or expression of an exogenous nucleic acid (e.g., DNA or RNA) in a cell in such a way as to allow expression of the coding portions of the introduced nucleic acid.

As used herein, the term “transfection” refers to the introduction of an exogenous and/or recombinant nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be in the form of naked DNA or RNA, it may be associated with various proteins or regulatory elements (e.g., a promoter and/or signal element), or the nucleic acid may be incorporated into a vector or a chromosome. A “transformed” cell is thus a cell transfected with a nucleic acid sequence. The term “transformation” refers to the introduction of a nucleic acid (e.g., DNA or RNA) into cells in such a way as to allow expression of the coding portions of the introduced nucleic acid. “Introducing” as described herein, in particular relating to nucleic acid sequences, can refer to any method of introducing nucleic acids into a cell as described herein or otherwise known in the art, for example, floral dipping (with A. tumefaciens strain GV301, for example), Agrobacterium-mediated methods, particle bombardment, electroporation, silicon carbide whiskers, microinjection, and/or nanoparticle-mediated delivery.

As used herein a “transformed cell” is a cell transfected with a nucleic acid sequence. As used herein, a “transgene” refers to an artificial gene or portion thereof that is used to transform a cell of an organism, such as a bacterium or a plant.

As used herein, “variant” can refer to a polynucleotide or polypeptide that differs from a reference polynucleotide or polypeptide but retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylations, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

As used herein, “isolated” means removed or separated from the native environment. Therefore, isolated DNA can contain both coding (exon) and noncoding regions (introns) of a nucleotide sequence corresponding to a particular gene. An isolated peptide or protein indicates the protein is separated from its natural environment. Isolated nucleotide sequences and/or proteins are not necessarily purified. For instance, an isolated nucleotide or peptide may be included in a crude cellular extract or they may be subjected to additional purification and separation steps.

With respect to nucleotides, “isolated nucleic acid” refers to a nucleic acid with a structure (a) not identical to that of any naturally occurring nucleic acid or (b) not identical to that of any fragment of a naturally occurring genomic nucleic acid spanning more than three separate genes, and includes DNA, RNA, or derivatives or variants thereof. The term covers, for example but not limited to, (a) a DNA which has the sequence of part of a naturally occurring genomic molecule but is not flanked by at least one of the coding sequences that flank that part of the molecule in the genome of the species in which it naturally occurs; (b) a nucleic acid incorporated into a vector or into the genomic nucleic acid of a prokaryote or eukaryote in a manner such that the resulting molecule is not identical to any vector or naturally occurring genomic DNA; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, e.g., a gene encoding a fusion protein, and (e) a recombinant nucleotide sequence that is part of a hybrid sequence that is not naturally occurring. Isolated nucleic acid molecules of the present disclosure can include, for example, natural allelic variants as well as nucleic acid molecules modified by nucleotide deletions, insertions, inversions, or substitutions.

As used herein, the term “vector” is used in reference to a vehicle used to introduce an exogenous nucleic acid sequence into a cell. A vector may include a nucleic acid molecule (e.g., a DNA or RNA polynucleotide), linear or circular (e.g., plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both. In embodiments of the present disclosure, vectors are derived from viruses capable of infecting one or more plant species can be referred to herein as plant viral vectors.

As used herein, “plasmid” refers to a type of vector that includes a non-chromosomal double-stranded DNA sequence including an intact “replicon” such that the plasmid is replicated in a host cell.

As used herein, the term “expression system” includes a biologic system (e.g., a cell based system) used to express a polynucleotide to produce a protein. Such systems generally employ a plasmid or vector including the polynucleotide of interest (e.g., an exogenous nucleic acid sequence, a recombinant sequence, etc.), where the plasmid or expression vector is constructed with various elements (e.g., promoters, selectable markers, etc.) to enable expression of the protein product from the polynucleotide. Expression systems use the host system/host cell transcription and translation mechanisms to express the product protein. Common expression systems include, but are not limited to, bacterial expression systems (e.g., E. coli), yeast expression systems, viral expression systems, animal expression systems, and plant expression systems.

As used herein, “wild-type” is the typical form of an organism, variety, strain, gene, protein, or characteristic as it occurs in nature, as distinguished from mutant forms that may result from selective breeding or transformation with a transgene.

The phrase “increased LDAP activity” means increased specific activity relative to that of the un-modified LDAP.

The term “plant” is intended to include a whole plant, any part of a plant, a seed, a fruit, propagules and progeny of a plant.

The term “propagule” means any part of a plant that may be used in reproduction or propagation, either sexual or asexual, including seeds and cuttings.

As used herein, “electroporation” is a transformation method in which a high concentration of plasmid DNA (containing exogenous DNA) is added to a suspension of host cell protoplasts, and the mixture shocked with an electrical field of about 200 to 600 V/cm.

As used herein, a “transgene” refers to an artificial gene which is used to transform a cell of an organism, such as a bacterium or a plant.

As used herein, the term “exogenous DNA, “Exogenous gene,” “exogenous nucleic acid sequence” or “exogenous polynucleotide” refers to a nucleic acid sequence that was introduced into a cell, organism, or organelle via transfection, electroporation, infiltration, or other delivery procedure. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

As used herein, the term “over-expression” and “up-regulation” or “increasing” production of a polypeptide refers to the expression of a nucleic acid encoding a polypeptide (e.g., a gene) in a modified cell at higher levels (therefore producing an increased amount of the polypeptide encoded by the gene) as compared to a “wild type” cell (e.g., a substantially equivalent cell that is not modified in the manner of the modified cell) under substantially similar conditions.

Conversely, “under-expression” and “down-regulation” refers to expression of a polynucleotide (e.g., a gene) at lower levels (producing a decreased amount of the polypeptide encoded by the polynucleotide) than in a “wild type” cell. As with over-expression, under-expression can occur at different points in the expression pathway, such as by decreasing the number of gene copies encoding for the polypeptide; removing, interrupting, or inhibiting (e.g., decreasing or preventing) transcription and/or translation of the gene (e.g., by the use of antisense nucleotides, suppressors, knockouts, antagonists, etc.), or a combination of such approaches. “Suppression” refers to the inhibition of production and/or activity functional gene product. Thus, the suppression of a gene or protein may indicate that the expression of the gene and/or activity of the encoded peptide has been inhibited such as by transcription and/or translation being inhibited, thus resulting in low to no production of the encoded protein, or production of a non-functional product, or production of an interfering nucleic acid that otherwise suppresses activity of the target protein.

Similarly, with respect to a gene product, such as a protein, “reduced activity” indicates that the activity of the protein is reduced relative to activity in a “wild type cell”. Such reduction in activity can be the result of inhibition/suppression/down-regulation/under-expression of the gene encoding the protein, the result of inhibition of translation of the messenger RNA into a functional gene product, or the result of production of a non-functional protein with reduced or no activity, or the direct suppression of the protein activity (e.g., preventing binding to a target), or the like. “Reduced production” of a gene product (e.g., a protein), such as by suppression, interruption, or other inhibition of transcription or translation, may result in reduced activity, but “reduced activity” of a protein or other gene product may result from other causes other than “reduced production”, such as set for the above.

Similarly, with respect to genes or other nucleic acids, “silencing” or “deletion” of a gene may include complete deletion of the nucleic acid/gene encoding a target peptide, complete suppression of translation or transcription of the target nucleic acid such that the target peptide is not produced, but the terms may also include some of the methods for “suppression” and “down-regulation” discussed above, where the “suppression” is significant enough to reduce expression of the target gene to the extent that the resulting peptide is inactive or the activity of the resulting peptide is so minimal as to be virtually undetected.

The term “null mutation” refers to a mutation in which the gene product (e.g., the protein encoded by the gene) is either not produced (or produced at significantly reduced levels, so as to be negligible) or is non-functional. Typically, a null mutation will involve a mutation of the native gene, such that the gene is not transcribed into RNA, the RNA product cannot be translated, or the protein produced by gene expression is non-functional.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Reference to “a” chemical compound refers to one or more molecules of the chemical compound rather than being limited to a single molecule of the chemical compound. Furthermore, the one or more molecules may or may not be identical, so long as they fall under the category of the chemical compound. Thus, for example, “a” chemical compound is interpreted to include one or more molecules of the chemical, where the molecules may or may not be identical (e.g., different isotopic ratios, enantiomers, and the like).

As used in the specification and the appended claims, the singular forms “a,” “an” and “the” include plural referents unless the context clearly dictates otherwise. Thus, for example, reference to “a metal oxide,” “an inert gas,” or “a catalyst,” includes, but is not limited to, two or more such metal oxides, inert gases, or catalysts, and the like.

Reference to “a/an” chemical compound, protein, and antibody each refers to one or more molecules of the chemical compound, protein, and antibody rather than being limited to a single molecule of the chemical compound, protein, and antibody. Furthermore, the one or more molecules may or may not be identical, so long as they fall under the category of the chemical compound, protein, and antibody. Thus, for example, “an” antibody is interpreted to include one or more antibody molecules of the antibody, where the antibody molecules may or may not be identical (e.g., different isotypes and/or different antigen binding sites as may be found in a polyclonal antibody).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, the term “effective amount” refers to an amount that is sufficient to achieve the desired modification of a physical property of the composition or material. For example, an “effective amount” of a filler refers to an amount that is sufficient to achieve the desired improvement in the property modulated by the formulation component, e.g. achieving the desired level of modulus. The specific level in terms of wt % in a composition required as an effective amount will depend upon a variety of factors including the amount and type of polycarbonate, amount and type of polycarbonate, amount and type of thermally conductive filler, and end use of the article made using the composition.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### B. INTRODUCTION

In accordance with the purpose(s) of the present disclosure, as embodied and broadly described herein, embodiments of the present disclosure, in some aspects, relate to compositions and methods for producing modified plants by modifying lipid droplet-associated protein (LDAP) genes in a plant to alter LDAP activity in the plant. The present disclosure also provides modified plants produced by the methods of modifying LDAP genes of the present disclosure and systems and methods for modifying plant genes. The present disclosure also provides genetically modified plants including an engineered modification including modified LDAP genes or, alternatively, an engineered modification including a mutation to the LDAP gene, seeds produced from these modified plants, and the like. Compositions, systems, and methods for modifying LDAP genes in a plant are also provided. Embodiments of such compositions and/or systems include, for example, a viral vector or a recombinant DNA plasmid to modify a LDAP gene in a plant or, alternatively, a site-directed mutagenesis to modify an amino acid or amino acids in a LDAP. Embodiments of such compositions, methods, plants, and systems facilitate and/or provide targeted genetic modifications to plant species without tissue culture.

Methods of the present disclosure involve targeted manipulation of LDAP expression to enhance packaging of wax esters into lipid droplets in order to increase wax ester collection from higher-yielding crops for commercial applications. This can provide the ability to address current and future challenges associated with lipid harvesting for commercial applications and advance agricultural technologies by informing future biotechnology strategies for the efficient packaging of various neutral lipid types as demonstrated in the current disclosure for wax esters (WEs) in transgenic seeds.

As described in greater detail in the examples below, since the jojoba plant expresses the unusual capacity to store liquid wax esters (WEs) in its seeds instead of triacylglycerols (TAGs) like most oilseed crops, jojoba was used to elucidate genetic pathways regulating WE packaging into lipid droplets (LDs). In the examples below, WE synthesis and packaging were reconstituted in leaves of Nicotiana benthamiana in order to screen jojoba proteins for the ability to support LD formation. The jojoba LDAP1 (ScLDAP1) protein was identified to be responsible for packaging WEs from the endoplasmic reticulum (ER) into LDs. It was shown that identification and disruption of this protein's activity would reduce WE packaging into LDs, whereas LDAP1 isoforms from other plants, such as Arabidopsis thaliana (AtLDAP1), did not support such WE partitioning from the ER into LDs, although both AtLDAP1 and ScLDAP1 were targeted specifically to LD monolayer surfaces.

As described in greater detail in the examples below, it was found that the selective, efficient WE partitioning by ScLDAP1 was facilitated by an amphipathic α-helix near the C-terminus of the protein, and mutational analysis identified one amino acid residue within this helix that facilitated proper WE packaging into cytoplasmic LDs. Manipulation of LDAP genes in order to facilitate such WE packaging into LDs provides methods of the present disclosure for modifying LDAP proteins in plants, modified plants produced by such methods, and systems and methods for producing modified plants.

### C. TECHNICAL CONTEXT

Storing lipids is a universal and evolutionarily conserved feature across organisms found in all of biology. While storage mechanisms may differ between prokaryotes and eukaryotes, one common theme is that hydrophobic molecules are packaged into subcellular organelles known as lipid droplets (LDs). In plants, LDs are generally small in size (i.e., ˜1.0 μm) and composed of a neutral lipid core surrounded by a phospholipid monolayer derived from the endoplasmic reticulum (ER) membrane during a directional budding process. In many organisms, the primary neutral lipids that are stored in LDs are triacylglycerols (TAGs) and stearyl esters (SEs), but in some organisms the core of the LDs can vary, including storing WEs, retinyl esters, or terpenes. Regardless of their composition, LDs were originally thought of as merely storage depots for lipophilic compounds but have since been found to be linked to a diverse array of functions including membrane trafficking, lipid signaling, and membrane remodeling during responses to stress and/or developmental changes.

While the overall mechanisms underlying LD biogenesis are still being uncovered, super-resolution microscopy and electron microscopy tomography have revealed that LD biogenesis begins at the ER membrane bilayer. Various proteins, including those involved in the final steps of neutral lipid synthesis, as well as various structural proteins specific to LD formation and maintenance, localize to distinct regions within the ER. It is at these sites that neutral lipid synthesizing enzymes (e.g., acyltransferases) function to increase the local concentrations of neutral lipids, such as TAGs within the ER bilayer. Once a specific concentration is reached, these non-bilayer-forming lipids demix and coalesce into what is referred to as a “lipid lens” between the two leaflets of the phospholipid bilayer. After the establishment of the lipid lens, various ER membrane bound structural proteins (referred to as class 1 type LD proteins) localize to and promote the protrusion of the lipid lens into the cytoplasm. In addition to the membrane-bound proteins, soluble proteins from the cytoplasm (referred to as class 2 type LD proteins) associate with and assist in stabilizing the growing, nascent LD. Although a consensus LD targeting sequence has yet to be identified, most class 2-type LD proteins utilize an amphipathic α-helix(s) to associate with the LD surface. While the role(s) of these LD structural proteins are still poorly understood, it is thought that some assist in the mitigation of membrane packaging defects, promotion of membrane curvature, and/or LD stabilization and prevention of LD-LD fusion. In addition, other proteins presumably promote the emergence and vectorial “budding” of the LD away from the ER surface, yielding a fully-formed, nascent LD that may be released into the cytoplasm via a scission event or may remain physically connected to the ER.

Across eukaryotes, some homologous proteins seem to be conserved in their involvement in LD biogenesis. One such protein, Seipin, which is an ER membrane protein, has been shown to facilitate site determination of LD formation, the directional emergence of the LD toward the cytoplasm, and for determining LD size and number. Other proteins such as the class 2-type perilipin proteins in mammals, also influence LD size and numbers, and also are involved in resolving packing defects at the surface of nascent LDs. Although no obvious perilipin homologues exist in plants, it seems that analogous proteins have evolved similar functions, including the oleosins and LD-associated proteins (LDAPs), to support the emergence and stability of LDs in plant cells.

While most plant LD biogenesis studies have focused primarily on the storage of TAGs, a number of plant species accumulate other types of lipid compounds, such as WEs, terpenes, or TAGs containing unusual fatty acids. Many of these compounds are of economic interest due to their potential usage in high-value nutraceutical or industrial applications. However, they are often produced in plants with low yields or other agronomic limitations. Investigations into the underlying mechanisms responsible for the synthesis and accumulation of these lipids have revealed that differential expression and/or evolutionarily diverged forms of lipid biosynthetic enzymes are often involved in their production. Numerous attempts have been made to express these genes in higher yielding platform crops, but unfortunately, amounts of the desired lipids are often well below that needed for commercial applications and sometimes have negative effects on overall yield or development. For example, studies attempting to over accumulate economically valuable lipid compounds, such as patchoulol and WEs, in transgenic plants had some detrimental impacts on seedling establishment and plant growth and development.

In an effort to identify factors that reduce the negative effects associated with WE production and accumulation, a tissue-specific proteomic and transcriptomic analysis of the WE-accumulating seed tissues of the plant Simmondsia chinensis, which is more commonly referred to as jojoba, were performed. These analyses revealed that along with gene transcripts encoding proteins well known to be involved in WE synthesis, such as wax synthase (WE), fatty acyl-CoA reductase (FAR), and fatty acid elongase (FAE), transcripts for a number of LD protein homologues were preferentially expressed in WE-accumulating seed tissues, with corresponding proteins accumulated on the LDs.

Based on these results, it was hypothesized that jojoba has evolved one or more LD packaging protein(s) to facilitate or be specific for the efficient packaging WEs into LDs. In one example of the present disclosure, several of these LD-associated proteins from jojoba were tested for their capacity to support WE packaging in a transient, heterologous system, namely Nicotiana benthamiana leaves, along with molecular dynamics simulations and mutagenesis studies to develop a mechanistic explanation for WE recognition and packaging. Among the proteins examined, jojoba LDAP1 (ScLDAP1) uniquely promoted the partitioning of WEs from the ER into LDs, and one amino acid residue in an amphipathic α-helix near the C-terminus of the ScLDAP1 protein was found to facilitate the efficient packaging of WEs into LDs. ScLDAP1 not only promoted the packaging of WEs into LDs in leaves but also corrected the defective compartmentation of WEs in transgenic Arabidopsis seeds designed to synthesize and accumulate jojoba-like WEs.

### D. NUCLEIC ACID CONSTRUCTS

Described herein are recombinant DNA constructs for altering lipid droplet-associated protein (LDAP) activity in a plant. In embodiments, a DNA construct can comprise an LDAP nucleotide sequence encoding an LDAP (i.e., a “LDAP nucleotide sequence”), or a truncated functional variant thereof.

In certain aspects, a DNA construct can further comprise a promoter operably linked to the LDAP nucleotide sequence.

In certain aspects, DNA constructs can comprise a terminator sequence operably linked at the LDAP nucleotide sequence downstream of the LDAP nucleotide sequence.

In certain aspects, a LDAP protein can be a chimera of an Arabidopsis LDAP1 (AtLDAP1) and a jojoba LDAP1 (ScLDAP1).

In certain aspects, a recombinant DNA construct described herein can further comprise one or more of a nucleotide sequence encoding an ScLDAP1, an N. benthamiana LDAP2 (NbLDAP2), an LDAP1, an LDAP2, an LDAP3, or a functional variant of any thereof, individually or in any combination of any thereof.

In certain aspects, a promoter can be one or more of Act1, HSP18.2, ScBV, RUBQ1, RUBQ2, CaMV35S, nos, P OsCon1, Ubi-1, MMV, SVBV, ocs, TBSV p19, TYLCV V2, and RbcS.

In certain aspects, a LDAP nucleotide sequence encoding the LDAP can comprise, consist essentially of, or consist of a sequence having 80% or greater sequence identity with SEQ ID NO:1. In certain aspects, a LDAP nucleotide sequence comprises SEQ ID NO:104 or SEQ ID NO:108.

In certain aspects, a LDAP nucleotide sequence can have at least 80% sequence identity with SEQ ID NO:1 or SEQ ID NO:33. In certain aspects, the recombinant DNA construct can comprise a LDAP nucleotide sequence encoding a protein comprising SEQ ID NO:105 or 109.

In certain aspects, the LDAP nucleotide sequence encodes a protein having at least 80% sequence identity with SEQ ID NO:26 or SEQ ID NO:34.

In certain aspects, DNA constructs described herein can further comprise one or more nucleotide sequences encoding a Marinobacter aquaeolei fatty acyl reductase (MaFAR), a jojoba wax synthase (WS), a Fat-Induced Transcript 2 (FIT2), a Mannosyl (alpha-1,6-) glycoprotein beta-1,2-N-acetylglucosaminyltransferase (MmDGAT2), a Nicotiana benthamiana 5-Epi-Aristolochene Synthase (NbEAS), a Histidine-Aspartic Acid-Glutamic Acid-Leucine (HDEL) peptide sequence, a jojoba Fatty Acyl-CoA Reductase (ScFAR), a Seipin1, a Seipin2, an LDAP-interacting protein, an oleosin1, an oleosin5, an oleosin5-like protein, a wax synthase (WS), a Fatty Acyl-CoA Reductase, a Fatty Acid Elongase, or a functional variant of any thereof, individually or in any combination of any thereof.

In certain aspects, a recombinant DNA construct described herein can further comprise a nucleotide sequence encoding a fluorescent protein, for example, green fluorescent protein or other fluorescent protein, such as one or more listed in the fluorescent protein database at FPbase.org.

### E. SYSTEMS FOR GENETIC MODIFICATION OF LDAP IN PLANTS

The present disclosure also provides systems for modifying meristem genes in a plant. In embodiments, the system includes one or more viral vectors or other nucleic acid delivery vehicle that can deliver a recombinant DNA construct described herein (such as that described in section D above) to a cell or plant described herein. The one or more viral vectors can include engineered RNA sequences and/or RNA-based signals configured to modify a LDAP gene in a plant. In other embodiments, the system includes one or more recombinant DNA plasmids to modify a LDAP gene in a plant or, alternatively, a site-directed mutagenesis to modify an amino acid or amino acids in a LDAP.

The genetic constructs (i.e., DNA constructs or recombinant DNA constructs) of the present disclosure comprise one or more polynucleotide sequences of the present disclosure and/or polynucleotides encoding polypeptides of the present disclosure, and may be useful for transforming, for example, plant organisms. The genetic constructs of the present disclosure are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987.

Genetic constructs for expression of genes in transgenic plants typically include promoters for driving the expression of one or more cloned polynucleotides, terminators and selectable marker sequences to detect presence of the genetic construct in the transformed plant.

The promoters suitable for use in the constructs of this present disclosure are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the present disclosure. Examples of constitutive plant promoters include the CaMV 35S promoter, the nopaline synthase promoter and the octopine synthase promoter, and the Ubi 1 promoter from maize. Plant promoters which are active in specific tissues respond to internal developmental signals or external abiotic or biotic stresses are described in the scientific literature. Exemplary promoters are described, e.g., in WO 02/00894 and WO2011/053169, which is herein incorporated by reference.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phosphotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenberg. Eds) Springer Verlag. Berline, pp. 325-336.

In some embodiments, the RNA sequences and/or RNA-based signals are selected from siRNA sequences, guide RNA sequences for CRISPR-mediated gene editing, aptamers, ribozymes, and riboswitches and/or the LDAP gene to be silenced or modified. The engineered RNA sequences and/or RNA-based signals can induce a mutation in the LDAP gene. In embodiments, the mutation is selected from, but not limited to, the following: silencing, deletion of all or a portion of a polynucleotide sequence of the LDAP gene, modification of a portion of a polynucleotide sequence of the LDAP gene, and replacement of the LDAP gene with an exogenous gene. In embodiments, the engineered RNA sequences and/or RNA-based signal comprises an siRNA sequence that silences an LDAP gene in the plant.

Although the silencing and/or genetic modification to the LDAP gene can be produced by various methods known to those of skill in the art of genetic manipulation of plants, in some embodiments, such as those described in greater detail in the Examples below, the engineered RNA sequences and/or RNA-based signal include a CRISPR/Cas system. For instance, in a non-limiting example, a system for modifying LDAP genes in a plant according to the present disclosure can include a CRISPR RNA guide sequence targeting the LDAP gene, such that binding of the CRISPR RNA guide sequence to the LDAP gene enables editing of the LDAP gene with a Cas nuclease (such as, but not limited to, Cas 9 and Cas 12a).

As discussed above, while various LDAP genes can be modified according to aspects of the present disclosure, in embodiments, the modified LDAP gene is ScLDAP1 from jojoba or a homolog (e.g., ortholog or paralog) thereof. For instance, the modified LDAP gene can have a polynucleotide sequence that is about 80-100% identical to ScLDAP1 from jojoba having SEQ ID NO:1.

In embodiments, the modified LDAP gene can be a chimera of the C-terminal region of ScLDAP1 and the N-terminal region of LDAP from other plants, such as Arabidopsis thaliana, such as a chimera having a C-terminus region with a protein sequence that is about 80-100% identical to SEQ ID NO:26.

Various viral vector systems can be used according to different aspects of the present disclosure. In some embodiments, the system includes one or more viral vectors. It may be advantageous to include both the gene silencing constructs and engineered RNA sequences/or RNA-based signals for modification of the LDAP gene all in a single vector for delivery to the recipient plant. However, in some embodiments of systems of the present disclosure, the constructs may be included in two or more viral vectors. In some embodiments, a first viral vector includes the siRNA targeting a LDAP gene in a plant, and a second viral vector includes the engineered RNA sequences and/or RNA-based signals configured to modify a LDAP gene in a plant. In some embodiments, the viral vecor is a single vector including the RNA for silencing the LDAP gene and the engineered RNA sequences or RNA-based signals to modify the LDAP gene.

### F. METHODS OF MODIFYING LDAP GENES IN A PLANT

Methods for modifying the sequence of proteins, or the polynucleotide sequences encoding them, are well known to those skilled in the art. The sequence of a protein may be conveniently modified by altering/modifying the sequence encoding the protein and expressing the modified protein. Approaches such as site-directed mutagenesis may be applied to modify existing polynucleotide sequences. Altered polynucleotide sequences may also be conveniently synthesized in its modified form. In certain aspects, methods as described herein can comprise introducing or otherwise delivering a recombinant DNA construct or system described herein to a cell or a plant by methods described herein and as known in the art.

Some embodiments of the present disclosure involve modifying endogenous LDAP polynucleotides to express the modified LDAP proteins of the present disclosure.

In embodiments, the modified LDAP gene can be, but is not limited to, ScLDAP1 and/or homologs/orthologs thereof.

The methods of the present disclosure are applicable to a wide variety of plant species and cultivars. For purposes of illustration, in embodiments, the plant can be but is not limited to the following: a cotton plant, a tobacco plant, other Malvaceae plants; Arabidopsis, canola and other Brassicaceae plants; and Simmondsia chinesis (jojoba), among others.

In some embodiments, the LDAP gene is ScLDAP1 from jojoba or an ortholog/homolog thereof. For instance, such as illustrated in the Examples below, the LDAP gene can also include functional homologs/orthologs of ScLDAP1 from jojoba that are in other plant species, such as a polynucleotide sequence that is about 80-100% identical to ScLDAP1 from jojoba having SEQ ID NO:1. In embodiments, the LDAP gene can be a chimera of the C-terminal region of ScLDAP1 and the N-terminal region of LDAP from other plants, such as Arabidopsis thaliana, such as a chimera having a protein sequence of a C-terminal domain that is about 80-100% identical to SEQ ID NO:26.

The LDAP gene can be manipulated/modified via a variety of genetic modification techniques, such as mutation/editing, substitution, and the like. In embodiments, genetically manipulating the LDAP gene editing the LDAP gene with delivery of RNA molecules and/or RNA-based signals, such as, for instance, guide RNA sequences for CRISPR-mediated gene editing, aptamers, ribozymes, and riboswitches. For instance, in some embodiments, genetically manipulating the LDAP gene is done by genetically modifying the LDAP gene with a CRISPR/Cas gene editing system including a CRISPR RNA guide sequence targeting the LDAP gene. The CRISPR RNA guides sequences can include one or more CRISPR RNA guide sequences targeting the LDAP gene and a Cas nuclease that delivered to plant cells in one or more viral vectors. The Cas nuclease can be selected from a wide variety of Cas nucleases (many are known and others are still being discovered). Examples of some possible Cas nucleases that could be used with methods of the present disclosure include, but are not limited to, Cas 9 and Cas 12a.

Methods for modifying endogenous genomic DNA sequences in plants are known to those skilled in the art. Such methods may involve the use of sequence-specific nucleases that generate targeted double-stranded DNA breaks in genes of interest. Examples of such methods for use in plants include: zinc finger nucleases (Curtin et al., 2011. Plant Physiol. 156:466-473; Sander, et al., 2011 Nat. Methods 8: 67-69), transcription activator-like effector nucleases or “TALENs” (Cermak et al., 2011, Nucleic Acids Res. 39: e82; Mahfouz et al., 2011 Proc. Natl. Acad. Sci. USA 108: 2623-2628; Li et al., 2012 Nat. Biotechnol. 30: 390-392), and LAGLIDADG homing endonucleases, also termed “meganucleases” (Tzfira et al., 2012 Plant Biotechnol. J. 10: 373-389).

Targeted genome editing using engineered nucleases such as clustered, regularly interspaced, short palindromic repeat (CRISPR) technology, is an important new approach for generating RNA-guided nucleases, such as Cas9, with customizable specificities. Genome editing mediated by these nucleases has been used to rapidly, easily and efficiently modify endogenous genes in a wide variety of biomedically important cell types and in organisms that have traditionally been challenging to manipulate genetically. A modified version of the CRISPR-Cas9 system has been developed to recruit heterologous domains that can regulate endogenous gene expression or label specific genomic loci in living cells (Sander and Young, Nature Biotechnology 32, 347-355 (2014)). The system is applicable to plants, and can be used to regulate expression of target genes. (Bortesi and Fischer, Biotechnology Advances Volume 33, Issue 1, January-February 2015, Pages 41-52).

In certain embodiments of the present disclosure, a genome editing technology (e.g., TALENs, a Zinc finger nuclease or CRISPR-Cas9 technology) can be used to modify one or more base pairs in a target endogenous LDAP gene or polynucleotide to create a codon encoding a Leucine (L) residue in the C-terminal region of the LDAP protein.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

A number of plant transformation strategies are available (e.g. Birch, 1997, Ann Rev Plant Phys Plant Mol Biol, 48, 297; Hellens et al., 2000, Plant Mol Biol 42: 819-32; Hellens et al., Plant Meth 1: 13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Transformation strategies may be designed to reduce expression of a polynucleotide/polypeptide in a plant cell, tissue, organ or at a particular developmental stage which/when it is normally expressed. Such strategies are known as gene silencing strategies.

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); Brassica (U.S. Pat. Nos. 5,188,958; 5,463,174 and 5,750,871); cereals (U.S. Pat. No. 6,074,877); pear (Matsuda et al., 2005, Plant Cell Rep. 24(1):45-51); Prunus (Ramesh et al., 2006 Plant Cell Rep. 25(8):821-8; Song and Sink 2005 Plant Cell Rep. 2006; 25(2):117-23; Gonzalez Padilla et al., 2003 Plant Cell Rep. 22(1):38-45); strawberry (Oosumi et al., 2006 Planta. 223(6):1219-30; Folta et al., 2006 Planta April 14; PMID: 16614818), rose (Li et al., 2003), Rubus (Graham et al., 1995 Methods Mol Biol. 1995; 44:129-33), tomato (Dan et al., 2006, Plant Cell Reports V25:432-441), apple (Yao et al., 1995, Plant Cell Rep. 14, 407-412), Canola (Brassica napus L.). (Cardoza and Stewart, 2006 Methods Mol Biol. 343:257-66), safflower (Orlikowska et al, 1995, Plant Cell Tissue and Organ Culture 40:85-91), ryegrass (Altpeter et al., 2004 Developments in Plant Breeding 11(7):255-250), rice (Christou et al., 1991 Nature Biotech. 9:957-962), maize (Wang et al., 2009 In: Handbook of Maize pp. 609-639) and Actinidia eriantha (Wang et al., 2006, Plant Cell Rep. 25, 5: 425-31). Transformation of other species is also contemplated by the present disclosure. Suitable methods and protocols are available in the scientific literature.

Targeted genome editing using engineered nucleases such as clustered, regularly interspaced, short palindromic repeat (CRISPR) technology, is an important new approach for generating RNA-guided nucleases, such as Cas9, with customizable specificities. Genome editing mediated by these nucleases has been used to rapidly, easily and efficiently modify endogenous genes in a wide variety of cell types and in organisms that have traditionally been challenging to manipulate genetically. A modified version of the CRISPR-Cas9 system has been developed to recruit heterologous domains that can regulate endogenous gene expression or label specific genomic loci in living cells (Nature Biotechnology 32, 347-355 (2014). The system is applicable to plants, and can be used to regulate expression of target genes. (Bortesi and Fischer, Biotechnology Advances Volume 33, Issue 1, January-February 2015, Pages 41-52). Use of CRISPR technology in plants is also reviewed in Zhang et al., 2019, Nature Plants, Volume 5, pages 778-794.

Methods of the present disclosure further include, after the above manipulation of a LDAP gene in a plant, then growing the modified plants, and identifying modified plants or plant sections with increased WE packaging into LDs, indicating the presence of modified genes, and collecting seeds from the plants. Embodiments further include growing the seeds to confirm retention of the increased WE packaging in subsequent generations.

An art skilled worker would know how to test the “increased WE packaging into LDs” activity of the chimeric LDAP. This may typically be done by isolating, enriching and quantifying the recombinant LDAP then using this material to determine the quantity of wax esters in the lipid droplets of a plant cell.

### G. GENETICALLY MODIFIED PLANTS WITH MANIPULATED LDAP GENE(S)

In addition to the above-described methods for genetically modifying plants, the present disclosure also provides genetically modified plants, such as plants made by the above-described methods. Such plants include an engineered genetic modification including a mutation to a LDAP gene or a genetic modification altering LDAP activity, where the genetically modified plant has increased WE packaging into LDs in comparison to WE packaging into LDs in a corresponding wild type plant.

The present disclosure further provides plant cells which comprise a genetic construct of the present disclosure, and plant cells modified to alter expression of a polynucleotide or polypeptide of the present disclosure, or used in the methods of the present disclosure. Plants comprising such cells also form an aspect of the present disclosure.

The present disclosure provides a host cell which comprises a genetic construct or vector of the present disclosure. Host cells comprising genetic constructs, such as expression constructs, of the present disclosure are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the present disclosure. Such methods may involve the culture of host cells in an appropriate medium in conditions suitable for or conducive to expression of a polypeptide of the present disclosure. The expressed recombinant polypeptide, which may optionally be secreted into the culture, may then be separated from the medium, host cells or culture medium by methods well known in the art (e.g., Deutscher, Ed, 1990, Methods in Enzymology, Vol 182, Guide to Protein Purification).

In one embodiment the modified LDAP protein has a greater capacity to increase cellular wax ester packaging from the endoplasmic reticulum to lipid droplets than does the unmodified LDAP protein.

The phrase “greater capacity to increase cellular wax ester packaging from the endoplasmic reticulum to lipid droplets” means that the modified LDAP of the present disclosure when expressed in a cell increases wax ester accumulation in lipid droplets, more than does the unmodified LDAP.

An art skilled worker would know how to test the “capacity to increase cellular wax ester packaging from the endoplasmic reticulum to lipid droplets” of the modified LDAP. This would typically involve expressing the modified LDAP in a cell, or cells, and expressing the unmodified LDAP in a separate cell, or cells of the same type. Wax ester accumulation in lipid droplets in the respective cells can then be assessed, for example, by methods well-known to those skilled in the art. An increase in wax ester accumulation in lipid droplets by expression of the modified LDAP protein relative to that by the unmodified protein, at the same time point, indicates that the modified LDAP protein has a “greater capacity to increase cellular wax ester packaging from the endoplasmic reticulum to lipid droplets” than does the unmodified LDAP protein.

The unmodified LDAP sequences, from which the modified LDAP sequences are produced, may be naturally-occurring LDAP sequences. In certain embodiments the cells into which the modified LDAP proteins are expressed are from plants. In other embodiments the modified LDAP proteins are expressed in plants.

In embodiments, the engineered mutation or alteration of LDAP activity is selected from, but not limited to: silencing, deletion of all or a portion of a polynucleotide sequence of the LDAP gene, modification of a portion of a polynucleotide sequence of the LDAP gene, and replacement of the LDAP gene with an exogenous gene.

Although various plant species and cultivars can be modified by methods of the present disclosure, in some embodiments, the plant is selected from the following: a cotton plant, a tobacco plant, and other Malvaceae plants; Arabidopsis, canola and other Brassicaceae plants; Simmondsia chinesis (jojoba), and other plant species.

As discussed above, while various LDAP genes from different species may be modified according to the present disclosure, in some embodiments, the modified LDAP gene is ScLDAP1 from jojoba or a homolog (e.g., ortholog thereof or paralog thereof) in another cultivar or species. In embodiments, the modified LDAP gene has a polynucleotide sequence that is about 80-100% identical to ScLDAP1 from jojoba having SEQ ID NO:1.

In embodiments, the modified LDAP gene can be a chimera of the C-terminal region of ScLDAP1 and the N-terminal region of LDAP from other plants, such as Arabidopsis thaliana, such as a chimera with a C-terminus region having a protein sequence that is about 80-100% identical to SEQ ID NO:26.

The plants of the present disclosure may be grown and either self-ed or crossed with a different plant strain and the resulting progeny, comprising the polynucleotides or constructs of the present disclosure, and/or expressing the modified LDAP sequences of the present disclosure, also form a part of the present disclosure.

Preferably the plants, plant parts, propagules and progeny comprise a polynucleotide or construct of the present disclosure, and/or express a modified LDAP sequence of the present disclosure.

The present disclosure also provides seeds produced by the genetically modified plants of the present disclosure.

Additional details regarding the methods, plants, seeds, and systems of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent.

It will be seen that aspects herein are well adapted to attain all the ends and objects hereinabove set forth together with other advantages which are obvious, and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Embodiments of the disclosure described above, and as described in the Examples below, include modification of an LDAP to express the ability of ScLDAP1 to package WEs from the ER into LDs.

### 1. Example 1—Materials and Methods

Embodiments of materials and methods according to the present disclosure as described herein the present example:

a. Plant Materials

Tobacco plants used in transient expression experiments were germinated and grown in soil at 28° C. under 16-h/8-h day night cycles. Leaves of 4-5-week-old Tobacco plants were infiltrated with the A. tumefaciens strain GV3101 harboring the appropriate binary vectors. For all infiltrations A. tumefaciens harboring the Tomato bushy stunt virus (TBSV) gene P19 were included to suppress gene silencing and enhance transgene expression, except for infiltrations that utilized N. benthamiana LDIP RNAi which instead utilized the Tomato yellow leaf curl virus (TYLCV) gene V2 to facilitate the suppression of gene silencing. A. tumefaciens transformation, growth, infiltration, and preparation of infiltrated material for microscopic analysis have been described previously in. For confirmation of both RNAi suppression as well as transgene expression, qualitative RT-PCR was used. Samples from mock infiltrated, as well as Agrobacterium infiltrate leaves were collected, flash frozen, and RNA extracted using the QIAGEN RNeasy Plant mini kit (QIAGEN, Inc., Valencia, CA) following manufacturer's protocol. 100 ng of RNA was then used to generate cDNA utilizing the SuperScript™ III First-Strand Synthesis System following manufacturers protocol. Approximately 50 ng of cDNA was then used as template sequences for PCR reactions to confirm the presence of transgene transcripts. PCR reactions were performed using Promega GoTaq® Green Master Mix (Promega, Madison, WI, USA) following the manufactures protocol. The following thermocycler protocol was used: 98° C. for 1 min, 35 amplification cycles (98° C. for 10 sec, 55° C. for 15 sec. 72° C. for 60 sec) and 72° C. for 7 min. Sequences of primers used in the reactions are listed in Sequences. Confirmation of NbLDIP RNAi suppression and transgene expression in N. benthamiana leaves is shown in FIGS. 14 and 15.

Experiments involving Arabidopsis employed wildtype (WT) Columbia (Col-0) ecotype, as well as transgenic lines overexpressing the genes involved in synthesis of WEs and/or jojoba LDAP1 generated from Col-0 ecotype. The Arabidopsis transgenic lines were obtained via floral dipping (Clough and Bent 1998) using A. tumefaciens strain GV3101 harboring binary vectors containing WE synthesizing, and jojoba LDAP1 genes. T1 plants with transgene expression were selected by spraying 7-day-old seedlings with 0.05% (w/v) Basta solution. T2 seeds were used for lipid analyses.

b. Plasmid Construction

Coding regions for the cloning of jojoba LD proteins and jojoba FAE were isolated from cDNA generated from jojoba seeds. RNA was purified from mature jojoba seeds. Approximately 100 ng of purified RNA was used to generate cDNA utilizing the SuperScript One-Step RT-PCR system (Invitrogen). jojoba WS, ScFAR, Seipin1, and Seipin2 were synthesized by the DNA synthesis services provided by GenScript Inc. Forward and reverse primers were utilized to amplify coding regions and are listed in Sequences. Restriction enzymes AscI and PacI (NEB) and T4 ligase (NEB) were used to insert amplified DNA into the plant expression vectors pMDC32 and pMDC32mCherry. Chimeric proteins were generated utilizing overlap extension PCR. Fragments used for overlap extension were amplified from vectors containing either AtLDAP1 or ScLDAP1 utilizing primers listed in Sequences. Fragments were then fused using an Overlap PCR reaction and cloned into the plant expression vector pMDC32mCherry using the restriction enzymes AscI and PacI (NEB), and T4 ligase (NEB). AtLDAP1 and ScLDAP1 mutants were synthesized by the DNA synthesis services provided by Gene Universal Inc. Genes were amplified using the appropriate forward and reverse primers from vectors containing synthesized genes, and cloned into pMDC32mCherry using the restriction enzymes AscI and PacI (NEB) and T4 ligase (NEB). The binary vectors for simultaneous expression of genes encoding Marinobacter aquaeolei fatty acyl reductase (MaFAR), S. chinensis wax synthase (WS), and S. chinensis LDAP1 (ScLDAP1) were generated using the Gateway technology (Thermo Fischer Scientific) as previously described. To construct the entry vectors, the coding sequences for WS, ScLDAP1, and ScLDAP1-GFP were amplified from plasmids pBinGlyRed-FWS3, pMDC32, and pMDC84, respectively, using Phusion High-Fidelity DNA polymerase (Thermo Fischer Scientific) and primers listed in Sequences. The amplified sequences were cloned into the SalI/BamHI restriction sites of either pENTRY-C5 (WS) or pENTRY-A4 (ScLDAP1, ScLDAP1-GFP). The resulting constructs were sequenced and used in combination with pENTRY-B6 containing YFP:myc:MaFAR to yield the binary vectors: pCAMBIA33-βcon::YFP:myc:MaFAR/gly::ScWS, pCAMBIA33-nap::LDAP1/βcon::YFP:myc:MaFAR/gly::ScWS, and pCAMBIA33-nap::LDAP1:GFP/βcon::YFP:myc:MaFAR/gly::ScWS. The correct assembly of the vectors was confirmed by sequencing.

c. Microscopy

Agrobacterium Infiltrated Tobacco leaves expressing heterologous genes were prepped for confocal microscopic analysis. Micrographs of N. benthamiana leaves were captured by using a Zeiss LSM710 confocal microscope retrofitted with an AiryScan detector head. Images were acquired with either a 63× oil immersion objective lens (NA=1.4), or a 40× water immersion objective lens (NA=1) and an Ar-ion laser (Carl Zeiss Inc.). Images of leaf cells were acquired as either Z-stacks (0.5 μm z-sections, 25 sections total) or as single optical plane images, and saved as 1,024×1,024-pixel digital images. AiryScan images were acquired as single optical plane images (0.25 μm thick) and saved as 1,240×1,240-pixel digital images. BODIPY 493/503 a neutral lipid specific dye and chlorophyll autofluorescence were excited with a 488 nm laser, CFP with a 405 nm laser, and mCherry with a 563 nm laser. Emission spectras for the above listed fluorophores were collected as follows: 500 to 540 for BODIPY fluorescence, 590 to 640 for mCherry, 450 to 490 for CFP, and 650 to 750 for chlorophyll autofluorescence. For neutral lipid visualization samples were stained with 2 μg/ml BODIPY 493/503 (prepared from a stock solution of 4 mg/ml dissolved in DMSO) in 50 mM PIPES buffer (pH 7). Images were taken from at least 2 separate infiltrated leaves across 3 individual infiltration experiments. Per sample, a minimum of 15 images were taken with representative images being shown. For total CFP fluorescence area quantification, the ImageJ 1.54f plugin 3D objects counter was used to quantify total CFP fluorescence area associated with ER defects. A minimum of 15 images across three infiltration experiments were used for CFP area quantification. All significance tests performed in this study were determined by one-way ANOVA with Tukey's post-test.

Transgenic seeds were prepared for confocal microscopic analysis of LDs by first imbibing dry seeds in water for 20-40 minutes to soften seed coat. Seed coats were then removed by gently rolling them between glass slide and cover slip. Seed embryos were then added to a fixative solution (4% paraformaldehyde in 50 mM PIPES pH 7.0) and incubated on a rotational shaker at 100 rpm for 20 minutes. Fixative solution was removed, and embryos washed three times with a solution of 50 mM PIPES pH 7.0 for 10 minutes each wash before being transferred to a dye solution (BODIPY 493/503 2 μg/ml in 50 mM PIPES pH 7.0) to stain for 20 minutes on a rotational shaker at 100 rpm covered from the light. Staining solution was removed, and embryos washed three times with a wash solution of 50 mM PIPES pH 7.0 for 10 minutes on a rotational shaker at 100 rpm covered from the light. Stained seed embryos were then transferred to slides and analyzed using a Zeiss LSM710 confocal microscope retrofitted with an AiryScan detector head. BODIPY 493/503 was excited by a 488 nm laser, and the emission signal was collected from 500 nm to 540 nm.

d. LD and Microsome Isolation

For LD and microsome isolations approximately 2.5 grams of infiltrated Tobacco leaves taken from plants 5 days post-infiltration were homogenized in 5 ml of ice-cold buffer 600 mM sucrose buffer (600 mM sucrose, NaH₂PO₄, pH 7.5, 150 mM NaCl, 0.1% (v/v) Tween-20, 1 mM PMSF, 1× cOmplete™ Protease Inhibitor Cocktail (Roche)). The resulting supernatant was then transferred to 15 ml centrifuge tubes. 2.5 ml of ice cold 400 mM sucrose buffer (400 mM Sucrose, 10 mM NaH₂PO₄, pH 7.5, 150 mM NaCl, 0.1% (v/v) Tween-20, 1 mM PMSF, 1× Complete™ Protease Inhibitor Cocktail (Roche)) was then layered on top of leaf homogenate and samples centrifuged at 10,500×g for 60 minutes at 4° C. to float LDs. The floated LDs were then carefully transferred to fresh tubes for either lipid extraction, or microscopic analysis, while the remaining supernatant was transferred to ultracentrifuge tubes and centrifuged and 100,000×g for 60 minutes at 4° C. The resulting supernatant was then removed, and the microsomal pellet resuspended in fresh 400 mM sucrose buffer and either used for lipid extractions or microscopic analysis.

e. Lipid Extraction and Analysis

For Cyclic FA extraction and analysis ˜100 mg of fresh leaves were submerged in 1.6 ml methanol. To this 200 μl of both toluene and 0.5 M sodium methoxide in methanol were added. 200 μg of a 15:0/15:0/15:0 TAG standard was added to this mixture, and then incubated at 65° C. for 30 minutes (vortexing for 5 seconds at ˜15 minutes) to generate FA methyl esters (FAME). Each sample was then quenched with 1 ml of 1% KCl (aq) and vortexed for 5 seconds. 1 ml of hexane was then added and vortexed for 10 seconds. Samples were then centrifuged for 5 minutes at 500×g. The upper organic phase was then transferred to fresh tubes and dried under a stream of N2. Samples were resuspended in 1 ml of hexane, and transferred to amber GC-MS vials prior to analysis by GC-MS.

For FAME analysis 1 μl of FAMEs were analyzed by GC-FID (Agilent 8890 GC system, equipped with a vf-23 ms capillary column (30 m×0.25 mm, 0.25 mm coating thickness; Agilent Technologies, Santa Clara, CA, USA). The oven program was as follows: 170° C. for 1 min, 5° C./min to 200° C., hold for 1 min, 2.5° C./min to 210° C., then finally 10° C./min to 240° C., holding for 3 minutes. Identification of peaks was based on retention times of standard FAMES, and extracts derived from CycFA producing plants (cotton, lychee). CycFAs were quantified by comparison against the C17 FAME peak response.

Sesquiterpene isolation and analysis were adapted from the methods listed in (Y. Cai et al., 2019). For terpene extraction from total tissues approximately 400 mg of infiltrated tissue was flash frozen in liquid nitrogen and terpenes extracted in 1 ml of CHCl₃. In addition, 200 nmols of the internal standard α-cedrene was added prior to incubation on a rotational shaker at room temp for 1 hour. Samples were then transferred to the fridge to incubate at 4° C. overnight. For lipid isolation from LD and microsomal fractions, 1 ml of CHCl₃ and 200 nmols of α-cedrene were added to each cellular fraction and vortexed vigorously for ˜30 seconds prior to centrifugation at 500×g for 10 minutes. For both the total tissue and cellular fractions the resulting organic phase was transferred to pre-washed SPE columns (Discovery™ DSC-Si SPE Tube) for separation of the polar lipid fraction from other neutral lipids. Neutral lipids were eluted by subsequent washes of 5 ml of 4:1 hexanes/diethyl ether and 1:1 hexanes/diethyl ether. Polar lipids were eluted into fresh test tubes by washing the column with 5 ml of 1:2 CHCl₃/MeOH. Samples were dried and derivatized with BSTFA by incubating at 50° C. for 1 hour. Samples were then dried one final time prior to resuspension in hexane and transferred to GC vials prior to analysis by GC-MS.

For capsidiol analysis, 1 μl of the derivatized lipids were analyzed by GC-MS (Agilent GC 7890A/MSD 5975C system equipped with a HP-5MS capillary column (30 m×0.250 mm, 0.25 mm coating thickness; Agilent Technologies, Santa Clara, CA, USA). The oven program temperature was as follows: 70° C. for 1 min, 20° C./min to 90° C., 8° C./min to 250° C., 30° C./min to 280° C., 280° C. for 5 min, 20° C./min to 300° C., and 300° C. for 1 min. Derivatized capsidiol was quantified by comparison against α-cedrene response under full mass scan mode.

For total lipid extraction approximately 5 grams of infiltrated Tobacco leaves taken from plants 5 days post-infiltration were flash frozen with liquid nitrogen, and then lyophilized overnight to remove water and prevent metabolic degradation of lipids by endogenous lipases. A portion of the lyophilized tissues (˜50-70 mg) were then measured into homogenization tubes prefilled with hexane-washed glass beads, and 40 nmols of both a WE standard heptadecanoyl heptadecanoate (17:0/17:0) (Nu-check Prep Inc.), and TAG standard glycerol triheptadecanoate (17:0/17:0/17:0) (Nu-check Prep Inc.) were added. A 70° C. solution of 2-propanal with the addition of 1% BHT (w/v) was added, and tissues disrupted for 1 minute using a Glen Mills Mini-Beadbeater-16. Homogenized tissue was transferred to new tubes and incubated at 70° C. for 30 minutes in a hot water bath. Samples were removed and allowed to cool to room temp before 1 ml of CHCl₃ was added with 400 μl of water to establish a ratio of solution of 2:1:0.45 2-propanol/CHCl₃/H₂0 (v/v/v) before being transferred to a 4° C. fridge overnight. Total lipids were separated into an organic layer through the addition of 1 ml of CHCl₃ and then washed 2× with 1 M KCl, with the aqueous layer removed after each wash. The organic layer was then transferred to new tubes, and concentrated under a stream of N₂, before being resuspended in 1 ml of CHCl₃. The resulting total lipid fraction was then added to SPE columns (Discovery™ DSC-Si SPE Tube) for separation of the neutral lipid fraction from polar lipids. For SPE fractionation of the neutral lipids, SPE columns were first washed with 5 ml of acetone, then conditioned with 2× washes of 5 ml of hexane. Total lipid fractions were then added and allowed to enter the column. For neutral lipid elution 6 ml of 4:1 hexanes/diethyl ether was added and washed through the column. The samples were then concentrated under a stream of N₂ and resuspended once more in 1 ml of CHCl₃ for storage before being prepped for MS analysis. For isolation of lipids from LD and microsomal fractions, lipids were extracted.

For MS analysis lipid extracts were dried under a stream of N₂ and then dissolved in 1 ml of methanol:chloroform (2:1, v/v) containing 5 mM ammonium acetate. Lipid extracts were analyzed on a Waters SYNAPT G2-si Mass Spectrometry System by direct infusion. Samples of each extract were analyzed using the following parameters: positive ionization mode, voltage of 2.5 kV, back pressure of 0.5 psi, source temperature of 40° C., and curtain gas set to 10 (arbitrary units). Data was collected using the MassLynx software. WE signals were selected using a 0.01-atomic mass unit window, a minimal signal-to-noise ratio of 1.0, and a maximum intensity of 0%. Data was processed using the mMass mass spectrometry tool. Samples were normalized against tissue dry weight, and relative quantification calculated based on the internal standard response. Confirmation of WE peaks was achieved using MS/MS fragmentation utilizing the same instrument and settings at a collision voltage of 5 kV, 10 kV, and 20 kV. Resulting fragmentations were then compared to published fragmentation patterns of WEs to confirm WE ID.

Lipid extraction from Arabidopsis seeds was performed. Briefly, 1.5 mg (for ESI-MS/MS analysis) or 5 mg (for GC-FID analysis) seeds were homogenized in 1 mL methanol with a glass rod in an 8 ml glass tube. After homogenization, 1 mL of chloroform was added to each sample. For ESI-MS/MS analysis, 5 nmol heptadecanoyl-heptadecanoate (Nu-Chek Prep, Inc., Elysian, MN, USA) was added to each sample as an internal standard. For GC-FID analysis, 50 μg di-17:0 WE and 100 μg tri-15:0 TAG were added. Lipids were extracted by shaking for 20 min at 4° C. Non-soluble cell fragments were pelleted by centrifugating at 450 g for 5 min. The supernatant was transferred into a new 8 ml glass tube, and the pellet was re-extracted with 1 mL n-hexane:diethyl ether:acetic acid (65:53:1, v/v/v) for 10 min at 4° C. with shaking. After centrifugation, the supernatants were combined and evaporated under streaming nitrogen. The lipid extracts were dissolved in 40 μl chloroform and separated by thin-layer chromatography (TLC) on 0.25×20×20 cm F60 silica gel glass plates using n-hexane:diethyl ether:acetic acid (80:20:1, v/v/v) as a solvent system. The lipid bands were visualized under UV light after spraying the plate with 0.2% (w/v) 8-anilino-1-naphthalenesulfonic acid or primuline (0.05% w/v in acetone/water, 80:20 v/v). For ESI-MS/MS measurement, WE bands were scraped from the plate and extracted twice with 1 ml n-hexane. The resulting supernatant was evaporated under streaming nitrogen. The WE fractions were dissolved in 2 ml methanol:chloroform (2:1, v/v) containing 5 mM ammonium acetate and diluted 100-fold. WE molecular species profiling was done according to the protocol described previously (Iven et al. 2013). For GC-FID analysis, WE and TAG bands were scraped from the plate, directly used for acidic methanolysis, and quantified.

f. Statistical Analysis

Statistical analysis of total CFP fluorescence associated with ER defects induced by WE accumulation were all performed using either ordinary one-way ANOVA test followed by Tukey's post-hoc multiple comparison test or one-way ANOVA followed by Kruskal-Wallis test using PRISM (v10.2.3) (GraphPad; www/graphpad.com). Statistical analysis of WE and TAG quantification from both total lipids, and isolated cellular fractions, were all performed using ordinary one-way ANOVA followed by Tukey's post-hoc multiple comparison test using PRISM (v10.2.3) (GraphPad; www/graphpad.com).

### 2. Example 2—Newly Synthesized, Very Long-Chain WEs Accumulate in the ER of N. benthamiana Leaves and their Partitioning into LDs is Ameliorated by Co-Expression of ScLDAP1

To assess the capacity of jojoba LD proteins for packaging WEs into LDs, the present disclosure reconstituted WE biosynthesis in N. benthamiana leaves via Agrobacterium-mediated ectopic-(co)expression of jojoba wax synthase (WS) and jojoba fatty acyl-CoA reductase (ScFAR). As shown in FIGS. 1A-1C, expression of both WS and ScFAR in N. benthamiana leaves revealed large, diffuse-stained, swollen-like structures within the ER, which was labeled with the ER marker protein CFP-HDEL, that co-localized with the neutral-lipid-specific dye BODIPY (493/503). To confirm that the BODIPY staining of ER defects was not a result of CFP-tagged recombinant protein overexpression, WS and ScFAR were co-expressed without CFP-HDEL and leaves were stained with BODIPY (FIG. 11). Similar large, diffuse structures were stained with BODIPY (arrows) indicating that the ER disruption most likely reflected neutral lipid accumulation (i.e., newly-synthesized WE) that was trapped in the ER rather than being properly partitioned into LDs.

To confirm that these ER structures were due to aberrant WE accumulation, and not due to protein aggregation from ectopic overexpression of the WS and FAR enzymes, N-terminal mCherry-tagged WS and ScFAR were co-expressed with either CFP-HDEL only, or with both CFP-HDEL and an untagged ScFAR or WS respectively. As shown in FIG. 1D, mCherry-WS expression with CFP-HDEL revealed that mCherry-WS localized in an expected manner to the ER and showed a normal reticulate-like ER pattern. Similarly, expression of both mCherry-WS and untagged ScFAR showed the same localization of mCherry-WS to the ER, and the same ER structures colocalizing with neutral lipid staining observed with WS and ScFAR co-expression (FIGS. 1A-1C), but no apparent aggregation of either protein at those ER defect sites (FIG. 1D). Expression of mCherry-ScFAR with CFP-HDEL showed cytoplasmic localization of ScFAR and normal reticulate ER structure (FIG. 1D). On the other hand, when mCherry-ScFAR and CFP-HDEL were expressed with untagged WS, the mCherry-ScFAR showed cytoplasmic localization, but, importantly, ER defects were observed and there was a lack of mCherry-ScFAR localization to the swollen ER regions (FIG. 1D). As expected WS and mCherry-ScFAR expression caused the appearance of swollen BODIPY stained structures (FIG. 1D, bottom row) similar to those seen when untagged WS and untagged ScFAR were expressed with CFP-HDEL (FIGS. 1A-1C). Taken together these results suggest that swollen regions of ER in WS and ScFAR expressing cells were caused by accumulation and improper WE release from the ER. This CLSM-based assay provided a means to visually survey jojoba proteins for their capacity to promote the proper partitioning of WEs from the ER into LDs.

Previous proteomic and transcriptomic analyses of developing jojoba seeds identified several candidate LD associated proteins that were enriched in WE-accumulating seed tissues. Of these, the present disclosure selected the following jojoba proteins for expression in N. benthamiana leaf cells, along with co-expressed untagged WS and ScFAR: Seipin1, Seipin2, LDAP1, LDAP3, LDAP-interacting protein (LDIP), oleosin1, oleosin5, and oleosin5-like. As shown in FIG. 2A, the majority of the jojoba proteins examined showed no noticeable reduction in the swollen regions of the ER, with the exception of ScLDAP1. Specifically, ScLDAP1 co-expression with WS and ScFAR appeared to ameliorate the ER disruptions and promote normal LD accumulation. LDAP1 proteins were shown previously to localize to the LD monolayer, and in transient or stable plant expression assays, promote the accumulation of LDs. Consistent with this, N-terminal mCherry-tagged ScLDAP1 (mCherry-LDAP1) co-expressed with untagged WS and ScFAR was localized specifically to LDs (based on its colocalization with BODIPY; FIG. 2B), and this was accompanied by an obvious reduction in ER swelling, i.e., compared to cells only co-expressing WS and ScFAR (FIGS. 1A-1D). Collectively, these results suggest that ScLDAP1 promotes the efficient partitioning of WEs from the ER into cytoplasmic LDs that are stabilized by the binding of ScLDAP1.

LDAP1 is a ubiquitous LD protein found in all plants that is predominately expressed in vegetative tissues, such as leaves and stems. However, in jojoba, LDAP1 is most abundantly expressed in developing seeds during WE accumulation and is enriched in the proteome of isolated LDs. To assess whether the reversal of the aberrant swollen ER phenotype in N. benthamiana leaves was specific to the LDAP1 from jojoba or rather was a property shared by LDAP1 isoforms from other plant species, N-terminal mCherry-tagged Arabidopsis LDAP1 (mCherry-AtLDAP1) was co-expressed with untagged WS and ScFAR. ER organization was then analyzed by CLSM based on the co-expressed ER marker protein CFP-HDEL and compared to jojoba mCherry-LDAP1. As shown in FIG. 3A, mCherry-AtLDAP1 localized to LDs similar to mCherry-ScLDAP1 but did not reverse the ER swelling or the release of the trapped neutral lipids. For a quantitative estimate of the restoration of the ER, the total fluorescent area of CFP-HDEL associated with the enlarged ER structures was measured and compared to control samples (i.e., empty vector [mock] and P19 viral suppressor of transgene silencing alone), or with the WS and ScFAR in the presence of either mCherry-ScLDAP1 or mCherry-AtLDAP1 (FIG. 3B). As anticipated from the visual CLSM results, mCherry-AtLDAP1 did not show a significant reduction in ER swelling area when compared to samples (co)expressing only WS and ScFAR, while mCherry-ScLDAP1 reduced the ER swelling to levels similar to leaves without WE production (CFP-HDEL alone; FIG. 3B). Similar ER swelling also was observed in cells expressing the native N. benthamiana LDAP1 (FIG. 12), reinforcing the notion that only ScLDAP1 was able to promote efficient WE exit from the ER to LDs. That is, it appeared that the functional activity of WE partitioning from the ER was unique to ScLDAP1 and not a characteristic shared with broader LDAP1 isoform family members.

To compare the total WE levels accumulated by co-expressing either AtLDAP1 or ScLDAP1, total neutral lipids were extracted and WE levels were quantified by direct-infusion, ESI-MS/MS (FIG. 3C). Overall, there was no difference in total WE content among any samples expressing the WE biosynthesis enzymes, regardless of whether an LDAP1 was co-expressed (FIG. 3C), suggesting that ScLDAP1 does not function to facilitate additional WE synthesis, but rather to promote more efficient partitioning of the synthesized WEs out of the ER and into LDs in these transient assays.

To corroborate in situ microscopy studies, a cell fractionation approach also was implemented to assess WE partitioning, whereby LDs from N. benthamiana leaf tissues were isolated by floatation centrifugation, and microsomes were pelleted from the remaining supernatant by ultracentrifugation (FIG. 4A). N. benthamiana leaves were infiltrated with WS, ScFAR, and jojoba FAE (to support fatty acid elongation for wax synthesis), with or without either AtLDAP1 or ScLDAP1. Then, LDs (marked by BODIPY) and microsomes (marked by CFP-HDEL) were isolated. Representative images of each of these fractions are shown in FIG. 4B. LDs were more abundant in LD fractions that had harbored either of the LDAP1s compared to mock or CFP-HDEL controls (FIG. 4B). It has been previously shown that overexpression of AtLDAP1 increased the number of LDs accumulated in transgenic Arabidopsis leaves, so it was not entirely unexpected that LDs would be induced/stabilized by either LDAP1 isoform. When comparing the microsomal fractions, a difference in the size and shape of CFP-HDEL-labeled ER-derived microsomes was obvious. That is, in samples expressing the WE biosynthesis enzymes alone, large CFP-HDEL containing structures were evident (FIG. 4B) similar in size to the swollen ER regions visualized in leaves (FIGS. 1A-1C, 2A, and 3A). Co-expression of AtLDAP1 yielded smaller sized CFP-HDEL structures, whereas the co-expression of ScLDAP1 resulted in the relative absence of these large structures from the microsomal fractions (FIG. 4B). Neutral lipids were quantified in these subcellular fractions and the amounts are reported in FIGS. 4C-4F. Overall, more WEs (on a tissue weight basis) were recovered in the LD fractions relative to microsome fractions in all samples that contained the WE synthesis machinery (left panel). However, the relative proportion remaining in the microsomes appeared to be greater in the absence of ScLDAP1 (FIGS. 4C-4F). That is, the co-expression of ScLDAP1 resulted in the most WEs recovered in the LDs, and the samples with AtLDAP1 appeared to recover significantly less WEs in LDs (FIGS. 4C-4F). Interestingly, the quantification of TAGs showed the reverse, whereby there was significantly more TAG in the LD fraction in the presence of AtLDAP1 compared to ScLDAP1 (or in the absence of either LDAP1) (FIGS. 4C-4F). Despite the caveats of losses of material during cell fractionation, it appears from the biochemical studies that the partitioning of WEs into LDs is promoted best by ScLDAP1. It is tempting to speculate from these results that ScLDAP1 has evolved to be more selective for the packaging of WEs and that the AtLDAP1 is more selective for TAGs.

### 3. Example 3—The Function of ScLDAP1 in WE Partitioning Depends on LDIP

A previous yeast two-hybrid interaction screen identified an Arabidopsis hydrophobic protein that directly interacts with LDAP proteins. This LDAP interacting protein (LDIP), was subsequently shown to interact with not only LDAPs but also SEIPIN proteins in Arabidopsis and facilitated the proper formation of LDs. Given this, it was suspected that the endogenous N. benthamiana LDIP (NbLDIP) might participate in the partitioning function of ScLDAP1. To test this possibility, an RNA interference (RNAi) assay previously used to suppress N. benthamiana LDIP expression in leaves was utilized, and to examine the effects of LDIP suppression in the WE reconstitution assays. As shown in FIG. 5A, LDIP suppression in leaves that were co-expressing WE biosynthetic enzymes and ScLDAP1 eliminated the corrective effect to the ER afforded by ScLDAP1. The ScLDAP1 function was confirmed to be LDIP-dependent based on CFP-fluorescent quantification associated with WE induced ER defects (FIG. 5B). ScLDAP1s capacity to more efficiently partition WEs into LDs was dependent on sufficient levels of endogenous LDIP, suggesting that WE-containing LD formation may proceed in an analogous manner to that of TAG-containing LDs except that ScLDAP1 has evolved features that make it specific to WEs.

### 4. Example 4—ScLDAP1 Utilizes a Specific Amphipathic Alpha-Helix for its WE Specificity

While results from molecular dynamics simulations indicated that there was a higher propensity for the identified loop of ScLDAP1 to bind to WE-filled LDs, the observed AtLDAP1 capacity to bind through the same region suggested that some other portion of ScLDAP1 can be responsible for facilitating WE partitioning out of the ER and into LDs. To test the interaction simulations, various embodiments of the present disclosure took advantage of the predicted, conserved multi-α-helix organization of the LDAP1 proteins with interrupted proline turns to swap peptide segments of the AtLDAP1 and the ScLDAP1 and evaluate the function of the resulting chimeric proteins (FIGS. 6A-6C). These chimeric proteins were then expressed in the N. benthamiana-based WE reconstitution assays to assess their capacity to promote proper WE exit from the ER. Initially, a pair of chimeric proteins were generated by swapping two halves of the protein: the N-terminal half of ScLDAP1 being replaced with the comparable region from AtLDAP1 (referred to as chimeric protein (CP) 1 and 2, respectively; FIG. 6A). Upon transient co-expression of either chimeric protein with the WE biosynthetic enzymes, only the chimeric protein (CP1) that contained the C-terminal half from ScLDAP1 had the ability to reduce the WE-induced swollen ER defects (FIG. 6A). To assess if the full C-terminal half of ScLDAP1 or one or more pieces of this protein segment were required for function, additional chimeric proteins (CP3, CP4, CP5, CP6) made by swapping smaller C-terminal regions, based around conserved LDAP α-helices were generated and tested in the same manner (FIG. 6A). Upon chimeric protein co-expression with the WE synthesizing machinery, confocal imaging revealed that neutral lipid release from the ER relied on the presence of an α-helix in the ScLDAP1 spanning residues 156 to 187 (FIG. 6A, 6B). This helix 6, colored yellow in the cartoon models of the LDAP proteins, sourced from ScLDAP1, but not from AtLDAP1, was sufficient to reduce the abundance of the swollen ER regions induced by WE synthesis. Chimeric protein production was confirmed by visualizing the mCherry fluorescence where the chimeric LDAPs each localized to LDs, further supporting separate functional regions on the LDAP proteins—one part for targeting to LDs which is the same for each LDAP1, and one that is selective for WE partitioning that is unique to the jojoba LDAP1 helix 6.

To assist in the identification of potential features from ScLDAP1 helix 6 that could potentially be responsible for the WE specific function, protein helical wheel projections and amino acid sequence alignments were compared for ScLDAP1 and AtLDAP1 (FIG. 6C). Helices from both LDAP1 proteins exhibited a strong amphipathic character, with similar overall hydrophobic and hydrophilic faces (FIG. 6C). Several residues were different between the two helices in terms of their position on the hydrophobic faces of the helices and the primary sequence (FIG. 6C). LD-associated protein analysis by others have suggested that large hydrophobic residues present on the non-polar face of amphipathic helices played a key role in preferential associations with LDs. Taking this into consideration, the residues 159 (F) and 162 (L) from jojoba were selected and the comparable residues 158 (1) and 161 (M) from Arabidopsis were selected to test for their function in facilitating the partitioning of WEs from the ER into LDs. N-terminal, mCherry-tagged single and double mutated versions of ScLDAP1 and AtLDAP1 coding sequences were generated and co-expressed in N. benthamiana leaves with the WE biosynthesis enzymes (i.e., untagged WS, ScFAR, FAE), as well as CFP-HDEL to visualize ER organization (FIGS. 7A and 7B). Mutations that swapped jojoba residues for Arabidopsis residues and vice versa included the following: ScLDAP1 F159I (ScL1-F159I), ScLDAP1 L162M (ScL1-L162M), ScLDAP1 F159I+L162M (ScL1-F159I/L162M), AtLDAP1 I158F (AtL1-I158F), AtLDAP1 M161L (AtL1-M161L), and AtLDAP1 I158F+M161L (AtL1I158F/M161L).

Converting the jojoba residue from F to I did not change its partitioning function; however, changing the L to M appeared to disrupt the protein's ability to reduce the ER swelling (FIG. 7A, compare the first two columns). The double mutant, ScL1F159I/L162M, also had disrupted function, due to it also carrying the L162M mutation. Further, the inverse mutations in the AtLDAP1 corroborated these results. The single mutant I158F in the Arabidopsis sequence did not alter the protein function from WT AtLDAP1; i.e., the ER defects were still visible (FIG. 7A, column 4). However, the single mutant M161L (and the double mutant containing the M161L) dramatically reduced the ER swelling and appeared to facilitate partitioning of neutral lipids out of the ER and into LDs in a manner similar to the jojoba native sequence (FIG. 7A, see last two columns). Enhanced resolution confocal images were collected from seven different leaf locations in each of three independent infiltration experiments and were quantified for CFP fluorescence (FIG. 7B). These combined data report a reproducible quantitative read-out of the ER swelling. The confirmation of mutant protein production was assessed in each experiment by mCherry fluorescence, where the mCherry-tagged LDAP1 proteins all co-localized with BODIPY staining of LDs. Taken together these results indicate that L162 within Helix 6 on ScLDAP1 is responsible for WE selectivity, and ultimately the ability to more efficiently partition WEs into LDs.

### 5. Example 5—Structural and Hydrophobic Differences Between Wildtype and Mutant LDAP1s

Both chimeric and site-directed mutation experiments point to a specific region of one amphipathic α-helix that appears to be responsible for ScLDAP1 WE selectivity. To seek a structural explanation for the differences in the helix 6, structural comparisons were performed between the AlphaFold predicted AtLDAP1 and ScLDAP1 helices. Comparing the AlphaFold predicted structures in this region highlighted differences in secondary structure between Arabidopsis and jojoba helices (FIGS. 8A-8F). In AtLDAP1, the secondary structure for residues between 159 to 163 is predicted to be a split helix-coil-helix motif. By comparison, in ScLDAP1 the secondary structure of helix 6 is predicted to be one continuous helix (FIG. 8C). By mutating the AtLDAP, M161L greater helicity was predicted (FIG. 8B). By visualizing the structure of the wildtype and mutant AtLDAP1 protein, the helicity breakage point is around the M161 residue. To classify any residue as part of an alpha helix by the visualization software the φ and ψ angles should be near −57° and −47° respectively. Plotting the φ and ψ angles on a Ramachandran plot for the wildtype Arabidopsis, mutant and wildtype jojoba shows that the dihedral angle of M161 is the largest change in this region (FIGS. 8A-8C).

Beyond the slightly modified helical angle predictions described above, the double mutant that introduces the two jojoba residues into the Arabidopsis sequence also changes the overall hydrophobicity of this region. As measured by summing the Whimley-White (WW) hydrophobicity scale values for the residues in the loop region, this specific place where the mutation is targeted is not particularly hydrophobic overall in AtLDAP1, with a sum score of +0.31 (FIG. 8D). By contrast, the jojoba LDAP1 is highly hydrophobic in this region, with a WW score of −0.99 (FIG. 8F). This negative score implies that the protein would be more favorable in this region to interact with a hydrophobic membrane environment. Just swapping the two jojoba residues into the AtLDAP1 protein (AtL1-I158F/M161L) was enough to change the WW score to negative (−0.84), similar to the native jojoba LDAP1 protein (FIG. 8E). These results suggest that the hydrophobicity of this specific loop region facilitates the selective interaction of ScLDAP1 with nascent LDs containing WEs and efficiently promotes the exit of WEs into LDs. It also explains why the AtLDAP1 is less effective in this functional role.

### 6. Example 6—Transgenic Arabidopsis Lines Co-Expressing ScLDAP1 with WE Biosynthetic Enzymes Show Improved Storage Lipid Compartmentalization

Previous studies for transgenic oil seed lines ectopically expressing WE synthesizing machinery, reported that WE accumulation led to deleterious side effects. Notably, one of these effects was disrupted neutral lipid packaging. Based on the results suggesting that ScLDAP1 supports WE partitioning out of the ER and into LDs, various embodiments of the present disclosure tested whether ScLDAP1 could improve the neutral lipid packaging in transgenic seeds producing WEs. Therefore, transgenic Arabidopsis lines were generated expressing either WS and Marinobacter aquaeolei FAR (MaFAR) by themselves, or co-expressed with untagged or C-terminal GFP-tagged ScLDAP1. Embryos from these transgenic lines were stained with BODIPY (493/503) and LD phenotypes visualized by enhanced-resolution confocal microscopy (Airyscan, FIG. 9A). Similar to what was reported previously, transgenic lines expressing just the WE machinery showed a disrupted neutral lipid packaging in the form of large lipid structures that somewhat resembled the ER defects observed in N. benthamiana reconstitution assays. Upon co-expression of either untagged- or GFP-tagged ScLDAP1 in transgenic Arabidopsis seeds, the disrupted lipid packaging was reversed to a more uniform LD population, similar to LD organization in seeds of the Col (0) non-transgenic background (FIG. 9A). WE and TAG levels within each transgenic line were also measured, and some but not all the lines expressing both ScLDAP1 and WE machinery showed increased WE accumulation when compared to the lines expressing only the WE machinery (FIG. 9B). However, expressing ScLDAP1 did not affect the profile of WE accumulated in the seeds (FIGS. 13A and 13B). These results indicate that overall, the ScLDAP1 is an important factor for effective WE packaging in plant cells.

### 7. Example 7—Jojoba (Simmondsia chinensis) Lipid Droplet-Associated Protein 1 Facilitates the Efficient Packaging of Wax Esters into Lipid Droplets

Within the last decade, the list of proteins and the understanding of their mechanistic contributions to LD biogenesis in plants have expanded considerably. Certainly, the abundant oleosin proteins have long been known to facilitate the stable compartmentalization of neutral lipids LDs in seeds and pollen, where cells undergo extreme desiccation and subsequent rehydration. However, a growing appreciation that LDs accumulate in many tissues that do not express oleosin genes, and the awareness of conserved protein-mediated mechanisms for lipid storage across kingdoms, has prompted a further examination of the potentially distinct ways in which LDs are produced and function in plants. This growing understanding has revealed an increasingly elaborate mechanism for LD biogenesis that involves the cooperation between several protein complexes to allow for the efficient formation and directional LD release from the ER into the cytoplasm. In plants, several central players have been shown to play roles in LD biogenesis, including SEIPINs, LDIP, LDAPs, and VAP27-1, which has provided a general model for how these fundamental components work together to produce LDs in all types of tissues and cells. Additional accessory proteins contribute to LD stability (oleosins), intracellular LD distribution (SLDP and LIPA), LD turnover (SDP1, PUX10, CDC48A, MIEL1), and LD function (caleosins, steroleosins), depending upon cellular demands, plant tissue types, and/or developmental stage. The present disclosure shows that there are other subtle, but important, variations in the fundamental LD biogenetic components that contribute to LD formation/function, depending upon the type of neutral lipid that is synthesized and packaged in LDs. In one aspect, the results show an amino acid residue in an amphipathic α-helix of LDAP1 from jojoba seeds (ScLDAP1) can be sufficient to support the efficient packaging of newly-synthesized WEs in a heterologous plant cell environment, supported by other regions of LDAP that bind to the LD surface. In work by others, transgenic oilseeds that were re-designed to synthesize WEs in their seeds showed aberrant LD morphologies with deleterious effects on seed viability and seedling growth. Given these atypical LD morphologies, it was proposed that the transgenic plants hosting the WE biosynthetic enzymes might lack the LD-related proteins utilized for efficient packaging of WEs into LDs. The results with Arabidopsis seeds co-expressing ScLDAP1 with the WE biosynthetic enzymes (FIGS. 9A-9C) support this concept and show that LD packaging proteins in general are an important consideration for biotechnology strategies aimed at producing large amounts of high-value, non-native lipids in heterologous plant systems.

a. ScLDAP1 Facilitates the Efficient Packaging of WEs into LDs

LD proteins have varied roles in the formation, stability, and degradation of LDs. Disruption of genes encoding most of these proteins has shown substantial impacts on lipid content, LD size, quantity, and/or location in vegetative and seed tissues. The transient expression assays reconstituting WE biosynthesis in N. benthamiana leaves offered both visual and biochemical readouts for the effective partitioning, or lack thereof, of neutral lipids out of the ER and into LDs (FIGS. 1-4). Even temporary overaccumulation of WEs resulted in disruptions in ER organization and the retention of neutral lipids in the ER rather than export to LDs, and this was only alleviated by co-expression of ScLDAP1 (FIGS. 2, 3). By contrast, the corresponding LDAP1 isoforms from Arabidopsis or N. benthamiana were ineffective at promoting the partitioning of the WEs out of the ER in these transient assays (FIGS. 3, 4, and 12).

Recent studies have shown that packing defects in the monolayer surface are sensed by amphiphilic α-helices of some LD proteins, with some also showing differential affinity towards different neutral lipid classes. For example, differential surface affinities by helical segments from various LD proteins were demonstrated for synthetic squalene-containing LDs, triolein-containing LDs, or sterol ester-containing LDs, indicating that packing defects at the monolayer surface, the underlying neutral lipid composition, and the nature of the amphipathic α-helix amino acids, all act to facilitate the association of LD proteins to monolayer rather than bilayer surfaces. Further, two-thirds of 442 plastoglobuli proteins identified in a proteomics study contain an amphipathic α-helix, which mediates the specific interaction of these proteins to the monolayer surface of the plastoglobuli and not to the bilayer surface of the thylakoid membrane. While a canonical LD targeting sequence has been difficult to generalize, it has been shown that for a majority of class 2 type LD proteins, localization to the LD is facilitated in part by the presence of amphipathic α-helices. Previous studies with LDAP1 from Arabidopsis showed that removing any part of the LDAP protein disrupted its localization to LDs in situ. Further, liposome-binding assays were insufficient to support a specific surface lipid-binding mechanism; however, no “monolayer” membrane was used to test the binding affinity of AtLDAP1, only different phospholipid compositions reflective of subcellular compartments (including LDs).

Studies show that protein-protein interactions at the surface via other LDAP proteins and/or the LDAP-interacting protein, LDIP, help to drive selectivity. Notably, LDAP proteins are known to interact with each other and also with LDIP, and the efficient WE partitioning out of the ER required LDIP (FIGS. 5A and 5B), so in one aspect the results are consistent with a protein-protein mediated mechanism. In another aspect, a selective WE-specific interaction(s) may occur over a longer time scale than the microsecond time frame simulated here. Consequently, an unbiased series of helix-swapping experiments followed by site-directed mutational analysis was implemented to gain further mechanistic insights into the nature of the selective partitioning activity of ScLDAP1 for WEs.

b. A Key Residue in an Amphipathic Alpha-Helix Confers WE Specificity to ScLDAP1 In Vivo

ScLDAP1 was predicted to be comprised largely of α-helices (e.g., FIGS. 8C and 8F; FIGS. 16A and 16B), and it was postulated that one or more of these helices in jojoba LDAP1 (ScLDAP1) may have uniquely evolved to have WE selectivity. Chimeric proteins comprised of various portions of ScLDAP1 and AtLDAP1 showed that one distinct amphipathic α-helix facilitated the proper packaging of WEs (FIGS. 6A-6C). Analyses by others showed that the residue composition of the hydrophobic face of amphipathic α-helices (as well as overall hydrophobicity of this face) determines the degree of affinity of an LD protein towards packing defects that occur during the formation of LDs, and that subtle changes in residue composition could dramatically alter this affinity. Based on the present results, increased hydrophobicity through a single residue mutation can be sufficient to allow AtLDAP1 to function like ScLDAP1 (FIGS. 7A and 7B). Introducing the mutation altered the predicted secondary structure in the AtLDAP1 helix to one more like that of ScLDAP (FIGS. 8B and 8E) which the results indicate affects residue positioning and ultimately association towards a WE-containing LD surface. In one aspect, the results indicate that a more hydrophobic helix might confer a more rapid association with a region of the ER where WEs are accumulating and promote a more efficient packaging process by accelerating binding of additional LDAP1 proteins or other protein partners, and/or a more pronounced curvature that would facilitate exit of WEs from the ER. In addition, the greater hydrophobicity of WEs compared to TAGs may highlight a need for an equally hydrophobic LD associating protein to stabilize the monolayer surface during early LD formation. The results indicate that a small evolutionary change in jojoba LDAP1 accompanied the plant's ability to efficiently store WEs in its seeds, a storage lipid class that is not widely found in other plants. Further, this change in ScLDAP1 hydrophobicity and functionality was a feature not revealed by simple sequence comparisons, but rather was context specific in an amphipathic α helix.

c. A Model for ScLDAP1 Function.

Taken together, the results are consistent with two separate regions that would allow for ScLDAP1 targeting and function. The first region suggests a conserved loop required for the initial targeting and insertion of LDAPs to a monolayer surface, while the region identified through mutational analysis is tuned towards facilitating WE partitioning from the ER to cytoplasmic LDs in jojoba. In one aspect the results are consistent with a model for the WE-specific function of ScLDAP1 (FIG. 10). Here, both AtLDAP1 and ScLDAP1 are depicted as capable of associating with, and binding to, WE-filled nascent LDs through a conserved N-terminal loop region shared between both proteins. Upon further WE accumulation within the ER bilayer and the appearance of packing defects on the nascent LD surface, the ScLDAP1 residues shown to be specific to WEs associate with and potentially stabilize those defects, thereby reducing membrane surface tension and promoting vectoral budding of the nascent LD. AtLDAP1, with its less hydrophobic region, is consequently less efficient at associating with and covering defects of the underlying WEs which in turn leads to higher surface tension and less efficient LD formation, resulting in retention of WEs in the aberrant, large lipid-stained structures trapped in the ER. Both AtLDAP1 and ScLDAP1 appear to be adequately suited to recognize similar defects that occur when just TAGs accumulate in LDs, and both proteins facilitate the release of TAG-filled LDs from the ER with equal efficiency (FIGS. 4A-F). In one aspect, the results are consistent with the model in FIG. 10. The results indicate that SEIPIN plays an important role in WE storage since LDIP appears to be required for ScLDAP1 function, and LDIP and SEIPIN are known to cooperate in the release of TAG-filled LDs from the ER.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

### I. REFERENCES

References are cited herein throughout using the format of reference number(s) enclosed by parentheses corresponding to one or more of the following numbered references. For example, citation of references numbers 1 and 2 immediately herein below would be indicated in the disclosure as (Refs. 1 and 2).
1. Al Sulaimi, R., Macknojia, A., Eskandari, M., Shirani, A., Gautam, B., Park, W., Whitehead, P., Alonso, A. P., Sedbrook, J. C., & Chapman, K. D. (2023). Evaluating the effects of very long chain and hydroxy fatty acid content on tribological performance and thermal oxidation behavior of plant-based lubricants. Tribology International, 185, 108576.
2. Antoine, G., Vaissayre, V., Meile, J.-C., Payet, J., Conéjéro, G., Costet, L., Fock-Bastide, I., Joët, T., & Dussert, S. (2023). Diterpenes of Coffea seeds show antifungal and anti-insect activities and are transferred from the endosperm to the seedling after germination. Plant Physiology and Biochemistry, 194, 627-637.
3. Bartels, D., & Dörmann, P. (n.d.). Plant Lipids.
4. Birsoy, K., Festuccia, W. T., & Laplante, M. (2013). A comparative perspective on lipid storage in animals. Journal of Cell Science, 126(7), 1541-1552.
5. Boerner, T. J., Deems, S., Furlani, T. R., Knuth, S. L., & Towns, J. (2023). ACCESS: Advancing Innovation: NSF's Advanced Cyberinfrastructure Coordination Ecosystem: Services & Support. Practice and Experience in Advanced Research Computing 2023: Computing for the Common Good, 173-176. https://doi.org/10.1145/3569951.3597559
6. Computing 2023: Computing for the Common Good, 173-176. https://doi.org/10.1145/3569951.3597559
7. Bouchnak, I., Coulon, D., Salis, V., D'Andréa, S., & Brehelin, C. (2023). Lipid droplets are versatile organelles involved in plant development and plant response to environmental changes. Frontiers in Plant Science, 14, 1193905.
8. Braun, R. J., & Swanson, J. M. J. (2022). Capturing the liquid-crystalline phase transformation: Implications for protein targeting to sterol ester-rich lipid droplets. Membranes, 12(10), 949.
9. Brooks, B. R., Bruccoleri, R. E., Olafson, B. D., States, D. J., Swaminathan, S. a, & Karplus, M. (1983). CHARMM: a program for macromolecular energy, minimization, and dynamics calculations. Journal of Computational Chemistry, 4(2), 187-217.
10. Cai, Y., Goodman, J. M., Pyc, M., Mullen, R. T., Dyer, J. M., & Chapman, K. D. (2015). Arabidopsis SEIPIN Proteins Modulate Triacylglycerol Accumulation and Influence Lipid Droplet Proliferation. The Plant Cell, 27(9), 2616-2636. https://doi.org/10.1105/tpc.15.00588
11. Cartwright, B. R., & Goodman, J. M. (2012). Seipin: from human disease to molecular mechanism. Journal of Lipid Research, 53(6), 1042-1055. https://doi.org/https://doi.org/10.1194/jlr.R023754
12. Chapman, K. D., Aziz, M., Dyer, J. M., & Mullen, R. T. (2019). Mechanisms of lipid droplet biogenesis. Biochemical Journal, 476(13), 1929-1942. https://doi.org/10.1042/BCJ20180021
13. Chen, J. C. F., Tsai, C. C. Y., & Tzen, J. T. C. (1999). Cloning and secondary structure analysis of caleosin, a unique calcium-binding protein in oil bodies of plant seeds. Plant and Cell Physiology, 40(10), 1079-1086.
14. Chorlay, A., & Thiam, A. R. (2020). Neutral lipids regulate amphipathic helix affinity for model lipid droplets. Journal of Cell Biology, 219(4), e201907099. https://doi.org/10.1083/jcb.201907099
15. Choudhary, V., & Schneiter, R. (2020). Lipid droplet biogenesis from specialized ER subdomains. Microbial Cell, 7(8), 218.
16. Chung, J., Wu, X., Lambert, T. J., Lai, Z. W., Walther, T. C., & Farese, R. V. (2019). LDAF1 and Seipin Form a Lipid Droplet Assembly Complex. Developmental Cell, 51(5), 551-563.e7. https://doi.org/https://doi.org/10.1016/j.devcel.2019.10.006
17. Clews, A. C., Ulch, B. A., Jesionowska, M., Hong, J., Mullen, R. T., &Xu, Y. (2023). Variety of plant oils: Species-specific lipid biosynthesis. Plant and Cell Physiology, pcad147.
18. Čopič, A., Antoine-Bally, S., Giménez-Andrés, M., La Torre Garay, C., Antonny, B., Manni, M. M., Pagnotta, S., Guihot, J., & Jackson, C. L. (2018). A giant amphipathic helix from a perilipin that is adapted for coating lipid droplets. Nature Communications, 9(1), 1332.
19. Curtis, M. D., & Grossniklaus, U. (2003). A gateway cloning vector set for high-throughput functional analysis of genes in planta. Plant Physiology, 133(2), 462-469.
20. Darden, T., York, D., & Pedersen, L. (1993). Particle mesh Ewald: An N·log(N) method for Ewald sums in large systems. The Journal of Chemical Physics, 98(12), 10089-10092.
21. de Vries, J., & Ischebeck, T. (2020). Ties between Stress and Lipid Droplets Pre-date Seeds. Trends in Plant Science, 25(12), 1203-1214. https://doi.org/https://doi.org/10.1016/j.tplants.2020.07.017
22. Deruyffelaere, C., Purkrtova, Z., Bouchez, I., Collet, B., Cacas, J.-L., Chardot, T., Gallois, J.-L., & D'Andrea, S. (2018). PUX10 is a CDC48A adaptor protein that regulates the extraction of ubiquitinated oleosins from seed lipid droplets in Arabidopsis. The Plant Cell, 30(9), 2116-2136.
23. Dhiman, R., Caesar, S., Thiam, A. R., & Schrul, B. (2020). Mechanisms of protein targeting to lipid droplets: A unified cell biological and biophysical perspective. Seminars in Cell & Developmental Biology, 108, 4-13. https://doi.org/https://doi.org/10.1016/j.semcdb.2020.03.004
24. Domergue, F., & Miklaszewska, M. (2022). The production of wax esters in transgenic plants: towards a sustainable source of bio-lubricants. Journal of Experimental Botany, 73(9), 2817-2834. https://doi.org/10.1093/jxb/erac046
25. Essmann, U., Perera, L., Berkowitz, M. L., Darden, T., Lee, H., & Pedersen, L. G. (1995). A smooth particle mesh Ewald method. The Journal of Chemical Physics, 103(19), 8577-8593.
26. Feller, S. E., Zhang, Y., Pastor, R. W., & Brooks, B. R. (1995). Constant pressure molecular dynamics simulation: The Langevin piston method. The Journal of Chemical Physics, 103(11), 4613-4621.
27. Gao, M., Huang, X., Song, B.-L., & Yang, H. (2019). The biogenesis of lipid droplets: lipids take center stage. Progress in Lipid Research, 75, 100989.
28. Gidda, S. K., Park, S., Pyc, M., Yurchenko, O., Cai, Y., Wu, P., Andrews, D. W., Chapman, K. D., Dyer, J. M., & Mullen, R. T. (2016). Lipid Droplet-Associated Proteins (LDAPs) Are Required for the Dynamic Regulation of Neutral Lipid Compartmentation in Plant Cells. Plant Physiology, 170(4), 2052-2071. https://doi.org/10.1104/pp. 15.01977
29. Gidda, S. K., Watt, S. C., Collins-Silva, J., Kilaru, A., Arondel, V., Yurchenko, O., Horn, P. J., James, C. N., Shintani, D., & Ohlrogge, J. B. (2013). Lipid droplet-associated proteins (LDAPs) are involved in the compartmentalization of lipophilic compounds in plant cells. Plant Signaling & Behavior, 8(11), e27141.
30. Giménez-Andrés, M., Čopič, A., & Antonny, B. (2018). The many faces of amphipathic helices. Biomolecules, 8(3), 45.
31. Gülz, P.-G., & Eich, C. (1982). Composition of Phospholipids in Seed Oil of Jojoba (Simmondsia chinensis [Link], Schneider). Zeitschrift Für Naturforschung C, 37(11-12), 1286-1287.
32. Guzha, A., Whitehead, P., Ischebeck, T., & Chapman, K. D. (2023). Lipid droplets: packing hydrophobic molecules within the aqueous cytoplasm. Annual Review of Plant Biology, 74, 195-223.
33. Hayes, D. G., Kleiman, R., & Phillips, B. S. (1995). The triglyceride composition, structure, and presence of estolides in the oils of Lesquerella and related species. Journal of the American Oil Chemists' Society, 72(5), 559-569.
34. Henne, M., Goodman, J. M., & Hariri, H. (2020). Spatial compartmentalization of lipid droplet biogenesis. Biochimica et Biophysica Acta (BBA)—Molecular and Cell Biology of Lipids, 1865(1), 158499.
35. Hilgarth, R. S., & Lanigan, T. M. (2020). Optimization of overlap extension PCR for efficient transgene construction. Methods X, 7, 100759.
36. Horn, P. J., James, C. N., Gidda, S. K., Kilaru, A., Dyer, J. M., Mullen, R. T., Ohlrogge, J. B., & Chapman, K. D. (2013). Identification of a new class of lipid droplet-associated proteins in plants. Plant Physiology, 162(4), 1926-1936.
37. Howell, S. H. (2013). Endoplasmic reticulum stress responses in plants. Annual Review of Plant Biology, 64, 477-499.
38. Huang, A. H. C. (2018). Plant lipid droplets and their associated proteins: potential for rapid advances. Plant Physiology, 176(3), 1894-1918.
39. Huang, C.-Y., & Huang, A. H. C. (2017). Unique motifs and length of hairpin in oleosin target the cytosolic side of endoplasmic reticulum and budding lipid droplet. Plant Physiology, 174(4), 2248-2260.
40. Huang, J., Rauscher, S., Nawrocki, G., Ran, T., Feig, M., De Groot, B. L., Grubmüller, H., & MacKerell Jr, A. D. (2017). CHARMM36m: an improved force field for folded and intrinsically disordered proteins. Nature Methods, 14(1), 71-73.
41. Humphrey, W., Dalke, A., & Schulten, K. (1996). VMD: visual molecular dynamics. Journal of Molecular Graphics, 14(1), 33-38.
42. Hunter, J. D. (2007). Matplotlib: A 2D graphics environment. Computing in Science & Engineering, 9(03), 90-95.
43. Ischebeck, T., Krawczyk, H. E., Mullen, R. T., Dyer, J. M., & Chapman, K. D. (2020). Lipid droplets in plants and algae: Distribution, formation, turnover and function. Seminars in Cell & Developmental Biology, 108, 82-93.
44. Ishikawa, T., Watanabe, N., Nagano, M., Kawai-Yamada, M., & Lam, E. (2011). Bax inhibitor-1: a highly conserved endoplasmic reticulum-resident cell death suppressor. Cell Death & Differentiation, 18(8), 1271-1278.
45. Ivarson, E., Iven, T., Sturtevant, D., Ahlman, A., Cai, Y., Chapman, K., Feussner, I., & Zhu, L.-H. (2017). Production of wax esters in the wild oil species Lepidium campestre. Industrial Crops and Products, 108, 535-542.
46. Iven, T., Hornung, E., Heilmann, M., & Feussner, I. (2016). Synthesis of oleyl oleate wax esters in Arabidopsis thaliana and Camelina sativa seed oil. Plant Biotechnology Journal, 14(1), 252-259. https://doi.org/https://doi.org/10.1111/pbi.12379
47. Jackson, C. L. (2019). Lipid droplet biogenesis. Current Opinion in Cell Biology, 59, 88-96.
48. Jo, S., Kim, T., Iyer, V. G., & Im, W. (2008). CHARMM-GUI: a web-based graphical user interface for CHARMM. Journal of Computational Chemistry, 29(11), 1859-1865.
49. Jumper, J., Evans, R., Pritzel, A., Green, T., Figurnov, M., Ronneberger, O., Tunyasuvunakool, K., Bates, R., Žídek, A., Potapenko, A., Bridgland, A., Meyer, C., Kohl, S. A. A., Ballard, A. J., Cowie, A., Romera-Paredes, B., Nikolov, S., Jain, R., Adler, J., . . . Hassabis, D. (2021). Highly accurate protein structure prediction with AlphaFold. Nature, 596(7873), 583-589. https://doi.org/10.1038/s41586-021-03819-2
50. Kohlmeyer, A., & Vermaas, J. (2017). TopoTools. Zenodo.
51. Krawczyk, H. E., Sun, S., Doner, N. M., Yan, Q., Lim, M. S. S., Scholz, P., Niemeyer, P. W., Schmitt, K., Valerius, O., & Pleskot, R. (2022). SEED LIPID DROPLET PROTEIN1, SEED LIPID DROPLET PROTEIN2, and LIPID DROPLET PLASMA MEMBRANE ADAPTOR mediate lipid droplet-plasma membrane tethering. The Plant Cell, 34(6), 2424-2448.
52. Kumari, R. M., Khatri, A., Chaudhary, R., & Choudhary, V. (2023). Concept of lipid droplet biogenesis. European Journal of Cell Biology, 102(4), 151362.
53. Laibach, N., Schmidl, S., Müller, B., Bergmann, M., Prüfer, D., & Schulze Gronover, C. (2018). Small rubber particle proteins from Taraxacum brevicorniculatum promote stress tolerance and influence the size and distribution of lipid droplets and artificial poly (cis-1, 4-isoprene) bodies. The Plant Journal, 93(6), 1045-1061.
54. Lanier, E. R., Andersen, T. B., & Hamberger, B. (2023). Plant terpene specialized metabolism: complex networks or simple linear pathways? The Plant Journal, 114(5), 1178-1201.
55. Lin, L.-J., Tai, S. S. K., Peng, C.-C., & Tzen, J. T. C. (2002). Steroleosin, a sterol-binding dehydrogenase in seed oil bodies. Plant Physiology, 128(4), 1200-1211.
56. Lundquist, P. K., Shivaiah, K.-K., & Espinoza-Corral, R. (2020). Lipid droplets throughout the evolutionary tree. Progress in Lipid Research, 78, 101029.
57. Miquel, M., Trigui, G., d'Andrea, S., Kelemen, Z., Baud, S., Berger, A., Deruyffelaere, C., Trubuil, A., Lepiniec, L., & Dubreucq, B. (2014). Specialization of oleosins in oil body dynamics during seed development in Arabidopsis seeds. Plant Physiology, 164(4), 1866-1878.
58. Miyamoto, S., & Kollman, P. A. (1992). Settle: An analytical version of the SHAKE and RATTLE algorithm for rigid water models. Journal of Computational Chemistry, 13(8), 952-962.
59. Naim, F., Nakasugi, K., Crowhurst, R. N., Hilario, E., Zwart, A. B., Hellens, R. P., Taylor, J. M., Waterhouse, P. M., & Wood, C. C. (2012). Advanced engineering of lipid metabolism in Nicotiana benthamiana using a draft genome and the V2 viral silencing-suppressor protein. PLoS One, 7(12), e52717.
60. Nettebrock, N. T., & Bohnert, M. (2020). Born this way—Biogenesis of lipid droplets from specialized ER subdomains. Biochimica et Biophysica Acta (BBA)—Molecular and Cell Biology of Lipids, 1865(1), 158448.
61. Ohlrogge, J., Thrower, N., Mhaske, V., Stymne, S., Baxter, M., Yang, W., Liu, J., Shaw, K., Shorrosh, B., & Zhang, M. (2018). Plant FA db: a resource for exploring hundreds of plant fatty acid structures synthesized by thousands of plants and their phylogenetic relationships. The Plant Journal, 96(6), 1299-1308.
62. Olzmann, J. A., & Carvalho, P. (2019). Dynamics and functions of lipid droplets. Nature Reviews Molecular Cell Biology, 20(3), 137-155.
63. Petrie, J. R., Shrestha, P., Liu, Q., Mansour, M. P., Wood, C. C., Zhou, X.-R., Nichols, P. D., Green, A. G., & Singh, S. P. (2010). Rapid expression of transgenes driven by seed-specific constructs in leaf tissue: DHA production. Plant Methods, 6, 1-6.
64. Phillips, J. C., Hardy, D. J., Maia, J. D. C., Stone, J. E., Ribeiro, J. V, Bernardi, R. C., Buch, R., Fiorin, G., Hénin, J., & Jiang, W. (2020). Scalable molecular dynamics on CPU and GPU architectures with NAMD. The Journal of Chemical Physics, 153(4).
65. Prévost, C., Sharp, M. E., Kory, N., Lin, Q., Voth, G. A., Farese Jr, R. V, & Walther, T. C. (2018). Mechanism and determinants of amphipathic helix-containing protein targeting to lipid droplets. Developmental Cell, 44(1), 73-86.
66. Prévost, C., Sharp, M. E., Kory, N., Lin, Q., Voth, G. A., Farese, R. V, & Walther, T. C. (2018). Mechanism and Determinants of Amphipathic Helix-Containing Protein Targeting to Lipid Droplets. Developmental Cell, 44(1), 73-86.e4. https://doi.org/https://doi.org/10.1016/j.devcel.2017.12.011
67. Pyc, M., Cai, Y., Gidda, S. K., Yurchenko, O., Park, S., Kretzschmar, F. K., Ischebeck, T., Valerius, O., Braus, G. H., & Chapman, K. D. (2017). Arabidopsis lipid droplet-associated protein (LDAP)-interacting protein (LDIP) influences lipid droplet size and neutral lipid homeostasis in both leaves and seeds. The Plant Journal, 92(6), 1182-1201.
68. Pyc, M., Cai, Y., Greer, M. S., Yurchenko, O., Chapman, K. D., Dyer, J. M., & Mullen, R. T. (2017). Turning over a new leaf in lipid droplet biology. Trends in Plant Science, 22(7), 596-609.
69. Pyc, M., Gidda, S. K., Seay, D., Esnay, N., Kretzschmar, F. K., Cai, Y., Doner, N. M., Greer, M. S., Hull, J. J., & Coulon, D. (2021). LDIP cooperates with SEIPIN and LDAP to facilitate lipid droplet biogenesis in Arabidopsis. The Plant Cell, 33(9), 3076-3103.
70. Qiu, B., & Simon, M. C. (2016). BODIPY 493/503 staining of neutral lipid droplets for microscopy and quantification by flow cytometry. Bio-Protocol, 6(17), e1912-e1912.
71. Rahman, F., Hassan, M., Rosli, R., Almousally, I., Hanano, A., & Murphy, D. J. (2018). Evolutionary and genomic analysis of the caleosin/peroxygenase (CLO/PXG) gene/protein families in the Viridiplantae. PLoS One, 13(5), e0196669.
72. Ramachandran, G. N., & Sasisekharan, V. (1968). Conformation of polypeptides and proteins. Advances in Protein Chemistry, 23, 283-438. https://api.semanticscholar.org/CorpusID:44890365
73. Renne, M. F., Klug, Y. A., & Carvalho, P. (2020). Lipid droplet biogenesis: a mystery “unmixing”? Seminars in Cell & Developmental Biology, 108, 14-23.
74. Romsdahl, T., Shirani, A., Minto, R. E., Zhang, C., Cahoon, E. B., Chapman, K. D., & Berman, D. (2019). Nature-guided synthesis of advanced bio-lubricants. Scientific Reports, 9(1), 11711.
75. Ruzicska, P., Gombos, Z., & Farkas, G. L. (1983). Modification of the fatty acid composition of phospholipids during the hypersensitive reaction in tobacco. Virology, 128(1), 60-64.
76. Sadre, R., Kuo, P., Chen, J., Yang, Y., Banerjee, A., Benning, C., & Hamberger, B. (2019). Cytosolic lipid droplets as engineered organelles for production and accumulation of terpenoid biomaterials in leaves. Nature Communications, 10(1), 853.
77. Santinho, A., Salo, V. T., Chorlay, A., Li, S., Zhou, X., Omrane, M., Ikonen, E., & Thiam, A. R. (2020). Membrane curvature catalyzes lipid droplet assembly. Current Biology, 30(13), 2481-2494.
78. Schindelin, J., Arganda-Carreras, I., Frise, E., Kaynig, V., Longair, M., Pietzsch, T., Preibisch, S., Rueden, C., Saalfeld, S., & Schmid, B. (2012). Fiji: an open-source platform for biological-image analysis. Nature Methods, 9(7), 676-682.
79. Scholz, P., Chapman, K. D., Mullen, R. T., & Ischebeck, T. (2022). Finding new friends and revisiting old ones-how plant lipid droplets connect with other subcellular structures. New Phytologist, 236(3), 833-838.
80. Sheinerman, F. B., & Brooks III, C. L. (1998). Molecular picture of folding of a small α/β protein. Proceedings of the National Academy of Sciences, 95(4), 1562-1567.
81. Shimada, T. L., Hayashi, M., & Hara-Nishimura, I. (2018). Membrane dynamics and multiple functions of oil bodies in seeds and leaves. Plant Physiology, 176(1), 199-207.
82. Shivaiah, K.-K., Boren, D. M., Herrera-Tequia, A., Vermaas, J., & Lundquist, P. K. (2023). An amphipathic helix drives interaction of Fibrillins with plastoglobule lipid droplets. BioRxiv, 2023-2029.
83. Siloto, R. M. P., Findlay, K., Lopez-Villalobos, A., Yeung, E. C., Nykiforuk, C. L., & Moloney, M. M. (2006). The accumulation of oleosins determines the size of seed oilbodies in Arabidopsis. The Plant Cell, 18(8), 1961-1974.
84. Smith, M. A., Zhang, H., Forseille, L., & Purves, R. W. (2013). Characterization of novel triacylglycerol estolides from the seed oil of Mallotus philippensis and Trewia nudiflora. Lipids, 48, 75-85.
85. Spangenburg, E. E., Pratt, S. J. P., Wohlers, L. M., & Lovering, R. M. (2011). Use of BODIPY (493/503) to visualize intramuscular lipid droplets in skeletal muscle. Journal of Biomedicine and Biotechnology, 2011.
86. Srivastava, R., Li, Z., Russo, G., Tang, J., Bi, R., Muppirala, U., Chudalayandi, S., Severin, A., He, M., & Vaitkevicius, S. I. (2018). Response to persistent ER stress in plants: a multiphasic process that transitions cells from prosurvival activities to cell death. The Plant Cell, 30(6), 1220-1242.
87. Sturtevant, D., Lu, S., Zhou, Z.-W., Shen, Y., Wang, S., Song, J.-M., Zhong, J., Burks, D. J., Yang, Z.-Q., & Yang, Q.-Y. (2020). The genome of jojoba (Simmondsia chinensis): A taxonomically isolated species that directs wax ester accumulation in its seeds. Science Advances, 6(11), eaay3240.
88. Szymanski, K. M., Binns, D., Bartz, R., Grishin, N. V, Li, W.-P., Agarwal, A. K., Garg, A., Anderson, R. G. W., & Goodman, J. M. (2007). The lipodystrophy protein seipin is found at endoplasmic reticulum lipid droplet junctions and is important for droplet morphology. Proceedings of the National Academy of Sciences, 104(52), 20890-20895.
89. Thiam, A. R., & Beller, M. (2017). The why, when and how of lipid droplet diversity. In Journal of Cell Science (Vol. 130, Issue 2). https://doi.org/10.1242/jcs.192021
90. Traver, M. S., & Bartel, B. (2023). The ubiquitin-protein ligase MIEL1 localizes to peroxisomes to promote seedling oleosin degradation and lipid droplet mobilization. Proceedings of the National Academy of Sciences, 120(29), e2304870120. https://doi.org/10.1073/pnas.2304870120
91. Thiam, A. R., & Ikonen, E. (2021). Lipid droplet nucleation. Trends in Cell Biology, 31(2), 108-118.
92. Van Der Walt, S., Colbert, S. C., & Varoquaux, G. (2011). The NumPy array: a structure for efficient numerical computation. Computing in Science & Engineering, 13(2), 22-30.
93. Vermaas, J. V, Petridis, L., Qi, X., Schulz, R., Lindner, B., & Smith, J. C. (2015). Mechanism of lignin inhibition of enzymatic biomass deconstruction. Biotechnology for Biofuels, 8, 1-16.
94. Vermaas, J. V, Rempe, S. B., & Tajkhorshid, E. (2018). Electrostatic lock in the transport cycle of the multidrug resistance transporter EmrE. Proceedings of the National Academy of Sciences, 115(32), E7502-E7511.
95. Vermaas, J. V, & Tajkhorshid, E. (2017). Differential membrane binding mechanics of synaptotagmin isoforms observed in atomic detail. Biochemistry, 56(1), 281-293.
96. Wimley, W. C., & White, S. H. (1996). Experimentally determined hydrophobicity scale for proteins at membrane interfaces. Nature Structural Biology, 3(10), 842-848.
97. Zhan, X., Zhang, Y.-H., Chen, D.-F., & Simonsen, H. T. (2014). Metabolic engineering of the moss Physcomitrella patens to produce the sesquiterpenoids patchoulol and α/β-santalene. Frontiers in Plant Science, 5, 636.
98. Zhao, Y., Dong, Q., Geng, Y., Ma, C., & Shao, Q. (2023). Dynamic Regulation of Lipid Droplet Biogenesis in Plant Cells and Proteins Involved in the Process. International Journal of Molecular Sciences, 24(8), 7476.
99. Zheng, F., Vermaas, J. V, Zheng, J., Wang, Y., Tu, T., Wang, X., Xie, X., Yao, B., Beckham, G. T., & Luo, H. (2019). Activity and thermostability of GH5 endoglucanase chimeras from mesophilic and thermophilic parents. Applied and Environmental Microbiology, 85(5), e02079-18.

What is claimed is:

1. A recombinant DNA construct for altering lipid droplet-associated protein (LDAP) activity in a plant, comprising:
an LDAP nucleotide sequence encoding an LDAP, or a truncated functional variant thereof;
a promoter operably linked to the LDAP nucleotide sequence; and
a terminator sequence operably linked at the LDAP nucleotide sequence downstream of the LDAP nucleotide sequence.

2. The recombinant DNA construct of claim 1, wherein the LDAP protein is a chimera of an Arabidopsis LDAP1 (AtLDAP1) and a jojoba LDAP1 (ScLDAP1).

3. The recombinant DNA construct of claim 1, further comprising a nucleotide sequence encoding an ScLDAP1, an N. benthamiana LDAP2 (NbLDAP2), an LDAP1, an LDAP2, an LDAP3, or a functional variant of any thereof.

4. The recombinant DNA construct of claim 1, wherein the promoter is selected from Act1, HSP18.2, ScBV, RUBQ1, RUBQ2, CaMV35S, nos, P OsCon1, Ubi-1, MMV, SVBV, ocs, TBSV p19, TYLCV V2, and RbcS.

5. The recombinant DNA construct of claim 1, wherein the LDAP nucleotide sequence encoding the LDAP comprises SEQ ID NO:1.

6. The recombinant DNA construct of claim 1, wherein the LDAP nucleotide sequence comprises SEQ ID NO:104 or SEQ ID NO:108.

7. The recombinant DNA construct of claim 1, wherein the LDAP nucleotide sequence has at least 80% sequence identity with SEQ ID NO:1 or SEQ ID NO:33.

8. The recombinant DNA construct of claim 1, wherein the LDAP nucleotide sequence encodes a protein comprising SEQ ID NO:105 or 109.

9. The recombinant DNA construct of claim 1, wherein the LDAP nucleotide sequence encodes a protein having at least 80% sequence identity with SEQ ID NO:26 or SEQ ID NO:34.

10. The recombinant DNA construct of claim 1 further comprising a nucleotide sequence encoding a Marinobacter aquaeolei fatty acyl reductase (MaFAR), a jojoba wax synthase (WS), a Fat-Induced Transcript 2 (FIT2), a Mannosyl (alpha-1,6-) glycoprotein beta-1,2-N-acetylglucosaminyltransferase (MmDGAT2), a Nicotiana benthamiana 5-Epi-Aristolochene Synthase (NbEAS), a Histidine-Aspartic Acid-Glutamic Acid-Leucine (HDEL) peptide sequence, a jojoba Fatty Acyl-CoA Reductase (ScFAR), a Seipin1, a Seipin2, an LDAP-interacting protein, an oleosin1, an oleosin5, an oleosin5-like protein, a wax synthase (WS), a Fatty Acyl-CoA Reductase, a Fatty Acid Elongase, or a functional variant of any thereof.

11. The recombinant DNA construct of claim 1 further comprising a nucleotide sequence encoding a fluorescent protein.

12. A method for genetically modifying a plant, comprising:
introducing a DNA construct of claim 1 to a plant or portion of a plant thereof, wherein the plant is a Solanaceae plant or a Brassicaceae plant; or
introducing at least one mutation in the nucleotide sequence of the C-terminal domain of the LDAP gene of the plant.

13. The method of claim 12, wherein the introducing is any one of floral dipping with A. tumefaciens strain GV301, Agrobacterium-mediated transformation, particle bombardment, or microinjection.

14. The method of claim 13, wherein the A. tumefaciens strain GV301 comprises a WS gene and an ScLDAP gene.

15. The method of claim 13, wherein the A. tumefaciens strain GV301 comprises an NbLDAP2 gene, an MmDGAT2 gene, a FIT2 gene, an NbEAS gene, or a combination of any thereof.

16. The method of claim 12, wherein the at least one mutation results in a leucine at a position corresponding to position 161 in an AtLDAP in the C-terminal domain of the LDAP protein of the plant.

17. A plant cell comprising a recombinant DNA construct of claim 1, further comprising a nucleotide sequence that encodes a lipid droplet-associated protein 1 (LDAP1) or a lipid droplet-associated protein 2 (LDAP2).

18. The plant cell of claim 17, further comprising a nucleotide sequence encoding a jojoba wax synthase (WS), a Marinobacter aquaeolei FAR (MaFAR), or a combination of any thereof.

19. The plant cell of claim 17, further comprising a nucleotide sequence encoding an MmDGAT2, an NbEAS, a FIT2, or a combination of any thereof.

20. A transgenic plant comprising a plurality of the plant cell of claim 17.
